SEQUENCE LISTING (54 sequences)

SEQ ID NO: 1 - RNA, 100 nt, synthetic construct
  source 1..100: mol_type = other RNA
acctgtagaacaaccaggtgatgttccacgtggacatctagcaggtgctgatcgagaaggactacttgaacgacatgctctagctctagatctacgtctt

SEQ ID NO: 2 - RNA, 100 nt, synthetic construct
  source 1..100: mol_type = other RNA
tgaaccacttctaggtggacttggtctagatcatgtagtacttctagctgttggtgatcgacctcaagttcaacaacttgatcctcttggtcatggtgga

SEQ ID NO: 3 - RNA, 100 nt, synthetic construct
  source 1..100: mol_type = other RNA
tcgacaagctccactagcaggtcatcatgttgcaccagcaccacaacttcatgtagttgtaggtgtaggtcatcgtcatctagtaccaggaccagaagat

SEQ ID NO: 4 - RNA, 100 nt, synthetic construct
  source 1..100: mol_type = other RNA
tggaccagtagttcgtcctgatcttgcagttcttgttgttgaaccagctgttctagctcgtcatgcagatggacgagttctaggactacttcgtcttcat

SEQ ID NO: 5 - RNA, 100 nt, synthetic construct
  source 1..100: mol_type = other RNA
aggtgttgatgtacatcctcatggagttcttggtccagctcaacgtgttcaacctcgagtaccagaacttcgaggtgtacgagaacgtcgtgttgttcat

SEQ ID NO: 6 - RNA, 100 nt, synthetic construct
  source 1..100: mol_type = other RNA
acgtcaacgtggacgtcctcgtgcaggtcgtgtaggtggtgttggtcaaccagcagaactaccaggagctggagctccagttgcacttcaaggagtagca

SEQ ID NO: 7 - RNA, 100 nt, synthetic construct
  source 1..100: mol_type = other RNA
acttggaggtgttgcaggtggtgcacaagttcctccacaagatgtacttggagaaccagctcgacttggtcttggacaacgtcgtgttctagcaggtcca

SEQ ID NO: 8 - RNA, 100 nt, synthetic construct
  source 1..100: mol_type = other RNA
aggtgttcctcaaggtggagatgtacatgtaggtcatccaggacttcttcatcctgttgttgatcgagtagttcatggtgtacctgttccagttgtacgt

SEQ ID NO: 9 - RNA, 100 nt, synthetic construct
  source 1..100: mol_type = other RNA
acaaggagttcatgcagctcttcaagatccagtaggagttcttgcacatcaagctcgtcttcatcaacatgttgatcttgatgaagcagttctacaacct

SEQ ID NO: 10 - RNA, 100 nt, synthetic construct
  source 1..100: mol_type = other RNA
cgatgctgtacgaggtggacttgatgatgatgcacatggtgctcctcctcgagctcttgtacgtggacttggagctgtaaatctaggtgaagctgaagct

SEQ ID NO: 11 - RNA, 100 nt, synthetic construct
  source 1..100: mol_type = other RNA
agaacttgcaccacgacatcgagctccaggtcgtcgtcatcatcgtcatggaggacttggtcgacgacgtctagtaggtggacgagaagaagcagttggt

SEQ ID NO: 12 - RNA, 100 nt, synthetic construct
  source 1..100: mol_type = other RNA
tcatcttcatgctctaggtcatgtagaaggaccagttcctcttgctgtagatcaaggacttcgtcaacctcgtgatgctcatgttgcacctggacgtgaa

SEQ ID NO: 13 - RNA, 100 nt, synthetic construct
  source 1..100: mol_type = other RNA
aggtcaagaagtacatcatgctgtacgaggtgaagatcaaggtgtacatgttctaccagttgctcttcgtgatggagaagctgtagctgttgctggtcta

SEQ ID NO: 14 - RNA, 100 nt, synthetic construct
  source 1..100: mol_type = other RNA
tgtactacatctactagtactacgtctaggtcctggtgtaggagctcttcgtggagttcgtctagtagatgcacgtgtagtacaagcagaacctcgagta

SEQ ID NO: 15 - RNA, 100 nt, synthetic construct
  source 1..100: mol_type = other RNA
aggagctgatggagttcttcatcttgatcgtcaacgtctacgtctaggtgtacgagaactagtagatcctcttgtacgagaacaacgtggagctcatgca

SEQ ID NO: 16 - RNA, 100 nt, synthetic construct
  source 1..100: mol_type = other RNA
tgtactagctgaagttgatcaagttgctgatcctggtctaggtggtctacttcatctagtaggacttctacttcgaggagatcatcgacaagcaccactt

SEQ ID NO: 17 - RNA, 100 nt, synthetic construct
  source 1..100: mol_type = other RNA
tcctcgtggaggtcgtgttgttgtactagttgcagttgtacgaggagatgcacgagttcgacatggagatgatggtgaacaacgtcgagttcatgatcta

SEQ ID NO: 18 - RNA, 100 nt, synthetic construct
  source 1..100: mol_type = other RNA
aggtcgtcaagatcgacctgaagtagtagttctagatgctgttgatcctcaagtagtaccagaactacttgttgtacgtcgtgctggtggactacgagga

SEQ ID NO: 19 - RNA, 100 nt, synthetic construct
  source 1..100: mol_type = other RNA
agttgttgcaggaccagttgcaggtcctgctggtgctctaggtcatcgaggacaagcagcacgtggacatggacttggagatgatgctgctcatgtactt

SEQ ID NO: 20 - RNA, 100 nt, synthetic construct
  source 1..100: mol_type = other RNA
tgttgatgcagtagatggtcgtgttgttgtagatggtgttggacgtgctgttgaaccacctgttctagttgaagttgatgcacgaccacaacatcaagca

SEQ ID NO: 21 - RNA, 100 nt, synthetic construct
  source 1..100: mol_type = other RNA
tgttcttcgtgatggtgttggtcgtgcacatccactagttcgtgtagctgctgatggtggaggtctacatcctcaaggacgacaagctgcagtacgtcgt

SEQ ID NO: 22 - RNA, 100 nt, synthetic construct
  source 1..100: mol_type = other RNA
tgttcgtgctggtgttcgacctcaacaagttgttcgtgaaggtgctgttgatccaccacatgctgctgctctaggtcatgatggtgaaggactagtagta

SEQ ID NO: 23 - RNA, 100 nt, synthetic construct
  source 1..100: mol_type = other RNA
tcgaggtgttcgagttcgtggtggtgttgaagctgttctacatcatgaacaacgtcttcctgttgtagaaggaggtcttggacaaggtcctgcaggagca

SEQ ID NO: 24 - RNA, 100 nt, synthetic construct
  source 1..100: mol_type = other RNA
acgaggagatgctcaagctggtgtacgtgttgatgttgtaggacatgtagtaggaggtgttccagatgctgcagctggtggtgttgctcgtggtcttgca

SEQ ID NO: 25 - RNA, 100 nt, synthetic construct
  source 1..100: mol_type = other RNA
acgtccacatgttggagatgttgttggacaagtacgagcagcacttgttcttggacgacttccacatcctgatgatcctcatgctgatgatggtgtacca

SEQ ID NO: 26 - RNA, 100 nt, synthetic construct
  source 1..100: mol_type = other RNA
tcctcaagcagatgttcttcttggtgcagttgttgttcttcaagttcatgaaggtggtgaagttgctgcagtacttcgtcttcgtcgtgctgtagtaggt

SEQ ID NO: 27 - RNA, 100 nt, synthetic construct
  source 1..100: mol_type = other RNA
tgttcttcatgtacaagctggaggtcgtgcagatggagatgcacgagatctacctggtgtagctgaagctcgtgatcaaggtggtgatccacatcctcgt

SEQ ID NO: 28 - RNA, 100 nt, synthetic construct
  source 1..100: mol_type = other RNA
tgatggtcgtgatcttgttcgagttcatcctgtagatggtgtacaagttgctgctctactacgacatcctcctgttccacgtgttgctgtactaggagta

SEQ ID NO: 29 - RNA, 100 nt, synthetic construct
  source 1..100: mol_type = other RNA
tgatcgacgtgctgcagctggagttgaagtagttcgtcgtctagttcttcatgttgttgctcaagtacgacctggtggtcgtggtcatctacctcttctt

SEQ ID NO: 30 - RNA, 100 nt, synthetic construct
  source 1..100: mol_type = other RNA
tgtagttcatgttgttgatcgtgcacgagaaggtggtcctggtcttattggtcttgttcaacgtcctcatgcaccagatcatcaacgagttgatcctggt

SEQ ID NO: 31 - RNA, 100 nt, synthetic construct
  source 1..100: mol_type = other RNA
tgtaccacatgttctagatggtcgtcttcttctacctgctgttgttcttgcacttgcagctcgtgcacgacttggtgaactacaagatgcagatgtagta

SEQ ID NO: 32 - RNA, 100 nt, synthetic construct
  source 1..100: mol_type = other RNA
tctacaacttggagatgctgctccagtagaaggtgctgttctagcagaacatggtccagaaggacatcgtctacttgtagttgttgtacatcgactactt

SEQ ID NO: 33 - RNA, 100 nt, synthetic construct
  source 1..100: mol_type = other RNA
agatgtagcactacatgttcaagttgtaccacttgatgttcttcgtgtacgtggtgttgctcctggaggtgttgatgtacaagatgtacaacttggtctt

SEQ ID NO: 34 - RNA, 100 nt, synthetic construct
  source 1..100: mol_type = other RNA
tcgagatcttctaccacatgtagttgctccagttcgtgaagatccagaacttgttgcaggagctgcactagtactacttcaactagatcatgatgcagga

SEQ ID NO: 35 - RNA, 100 nt, synthetic construct
  source 1..100: mol_type = other RNA
tgttccagttcatcaaggtcatcctcctctagtagatgatggagttgctgatccagtaccaggtgtaggagtacgacttgctcgacctggtgaacgtcct

SEQ ID NO: 36 - RNA, 100 nt, synthetic construct
  source 1..100: mol_type = other RNA
tgtaggaggtgtaggtccacaaggtgtacaagtacgtggtccacttgttgtactagatggtgtacatcttgtagctgatggagcactacttgcagcagat

SEQ ID NO: 37 - RNA, 100 nt, synthetic construct
  source 1..100: mol_type = other RNA
tcgtggtcctgttgttctacgagcactacgtgtaggacatggagctgaacttgttggagatggagcactacatggtcctcttcatggtgtacgtgcacaa

SEQ ID NO: 38 - RNA, 100 nt, synthetic construct
  source 1..100: mol_type = other RNA
tgtagaaggagctcatcttgctcaacctgttgtagtagcaggacttctacgacatcgtctacaacttcatcctggtcatcttctagctgttgcacttcca

SEQ ID NO: 39 - RNA, 100 nt, synthetic construct
  source 1..100: mol_type = other RNA
tccagtacctgctgttggactacatggacctgtacgtgatcaagtacatgcagaaggtcctgtagtacctcttggtgctggtcctcaagatgatcgtgga

SEQ ID NO: 40 - RNA, 100 nt, synthetic construct
  source 1..100: mol_type = other RNA
agctgtaggtcgtcgtgctgtacatgaagcaggtggtggtgtacgaggtcgactaccagatggacgacgacaaccagttcgagctcgtgctgcaggtgtt

SEQ ID NO: 41 - RNA, 100 nt, synthetic construct
  source 1..100: mol_type = other RNA
tcatgttcctggtggtcatctacatgatgtactagttcaagaagtacgtcctcttcatccacgagaaggtcgtcaaccacaacgtcttgtagcagctgat

SEQ ID NO: 42 - RNA, 100 nt, synthetic construct
  source 1..100: mol_type = other RNA
agttgctcaaggacgagctccacaagaagttcgtgttcaagatgtagtaggagatgatgctgctccagatgatccagtacaacctggacgtcgtggactt

SEQ ID NO: 43 - RNA, 100 nt, synthetic construct
  source 1..100: mol_type = other RNA
tcatcgacgaggagcagatgatcaaggtgtacaacgtcatcgtctagtacgagaagttctacgagtagatcatggtcttgttcttgaagttcaacatgtt

SEQ ID NO: 44 - RNA, 100 nt, synthetic construct
  source 1..100: mol_type = other RNA
tcaaccacgtccactacttcttcatggtcttgtagttgtacttcttgctgctcttgctcgtcaagttggtggtgttcgtgttcgagctggagctcgagat

SEQ ID NO: 45 - RNA, 100 nt, synthetic construct
  source 1..100: mol_type = other RNA
acaagtagtacaactacttcgtcgtgttctagatcgaccactacttgttgttctaggtcgacctctactacgacatcgtgctgcacgacaaggtcaagta

SEQ ID NO: 46 - RNA, 100 nt, synthetic construct
  source 1..100: mol_type = other RNA
tgatgtagctcatcatgatcttcgtcaaggtgaaggtcatgtagttgttgcacgacttcgtggagcacttgatggacaacttcgacgagaacttctagga

SEQ ID NO: 47 - RNA, 100 nt, synthetic construct
  source 1..100: mol_type = other RNA
aggtgttgatcctgctccacctcatcgtgctcctggtctagaagttgaacaagaacatcaacaagctcttcttgaacaacgtctagttgttcttggtaca

SEQ ID NO: 48 - RNA, 100 nt, synthetic construct
  source 1..100: mol_type = other RNA
tccacctgaagttcgtcatccactacatccacgtcttgatgaacctccacaacgtcatgttcgtgatgttcctgcagatccaggtggagctgtaggtcga

SEQ ID NO: 49 - RNA, 100 nt, synthetic construct
  source 1..100: mol_type = other RNA
acttcgagttcttcctcttggtcaagaagatcgtgttcttcttgatgtagaagttccactacatcgtggtggagtacgagatcctccaccagttgaagga

SEQ ID NO: 50 - RNA, 100 nt, synthetic construct
  source 1..100: mol_type = other RNA
tcctgcactaccacctcttcgacttcaacctggtcttgcagtagttcatgttggacgacctcttcttggtctacgagtacgtcatgttcttcaagtagta

SEQ ID NO: 51 - RNA, 18 nt, synthetic construct
  source 1..18: mol_type = other RNA
aaggaaaaaagcggccgc

SEQ ID NO: 52 - RNA, 151 nt, synthetic construct
  source 1..151: mol_type = other RNA
ttcgagcagacatgataagatacattgatgagtttggacaaaccacaactagaatgcagtgaaaaaaatgctttatttgtgaaatttgtgatgctattgctttatttgtaaccattataagctgcaataaacaagttaacaaccgctgccg

SEQ ID NO: 53 - RNA, 33 nt, synthetic construct
  source 1..33: mol_type = other RNA
acactctttccctacacgacgctcttccgatct

SEQ ID NO: 54 - RNA, 53 nt, synthetic construct
  source 1..53: mol_type = other RNA
gtgactggagttcagacgtgtgctcttccgatctcctccccgcatcgataccg

(19) United States
(12) Patent Application Publication
(10) Pub. No.: US 2025/0333761 A1
(43) Pub. Date: Oct. 30, 2025

(54) COMPOSITIONS AND METHODS FOR MULTIPLEX PROJECTION TRACING AND MULTI-MODAL PROFILING OF PROJECTION NEURONS USING PROJECTION-TAGs

(71) Applicants: Vijay Samineni, St. Louis, MO (US); Lite Yang, St. Louis, MO (US); Hannah Hahm, St. Louis, MO (US); Fang Liu, St. Louis, MO (US)

(72) Inventors: Vijay Samineni, St. Louis, MO (US); Lite Yang, St. Louis, MO (US); Hannah Hahm, St. Louis, MO (US); Fang Liu, St. Louis, MO (US)

(73) Assignee: Washington University, St. Louis, MO (US)

(21) Appl. No.: 19/188,928

(22) Filed: Apr. 24, 2025

Related U.S. Application Data
(60) Provisional application No. 63/638,021, filed on Apr. 24, 2024.

(51) Int. Cl. C12N 15/86; C07K 14/435; C07K 14/47; C12Q 1/6841; C12Q 1/6869

(52) U.S. Cl. CPC C12N 15/86; C07K 14/43595; C07K 14/47; C12Q 1/6841; C12Q 1/6869; C07K 2319/09; C12N 2750/14143; C12N 2830/48

(57) ABSTRACT

Projection-TAGs and methods of use thereof are provided, enabling comprehensive mapping of neuronal projections in the brain including multi-modal profiling and multiplex projection tracing. Embodiments of a Projection-TAG comprise an AAV plasmid including a promoter, an RNA barcode, a fluorescent marker, and a regulatory element. Embodiments of methods for multiplex tracing of a projection neuron in a brain of a subject in need thereof include administering a Projection-TAG to the subject, obtaining one or more biological samples from the subject, and applying an imaging modality to the one or more biological samples.

### CROSS-REFERENCE TO RELATED APPLICATIONS

This application claims the benefit of priority to U.S. Provisional Application Ser. No. 63/638,021 filed 24 Apr. 2024, which is incorporated herein by reference in its entirety.

### STATEMENT OF FEDERALLY SPONSORED RESEARCH OR DEVELOPMENT

This invention was made with government support under DK128475 and DA056829 awarded by the National Institutes of Health. The government has certain rights in the invention.

### MATERIAL INCORPORATED BY REFERENCE

The Sequence Listing, which is a part of the present disclosure, includes a computer-readable form comprising nucleotide and/or amino acid sequences of the present invention (file name “020975-US-NP_Sequence-Listing.xml” created 24 Apr. 2025; 52,915 bytes). The subject matter of the Sequence Listing is incorporated herein by reference in its entirety.

### FIELD

The present disclosure generally relates to comprehensive mapping for brain-wide neuronal cell projection via Projection-TAGs.

### BACKGROUND

Single-cell multiomic techniques have sparked immense interest in developing a comprehensive multimodal map of diverse neuronal cell types and their brain-wide projections. However, investigating the spatial organization, transcriptional and epigenetic landscapes of brain wide projection neurons is hampered by the lack of efficient and easily adoptable tools. Traditional neuroanatomical tracing methods, performed often with dual color fluorescent tracers or viral vectors, have been invaluable in mapping distinct neuronal projections. Furthermore, such approaches are not suited for high-throughput sequencing-based assays such as single-cell RNA sequencing (scRNAseq) and single-cell assay for transposase-accessible chromatin with sequencing (scATAC-seq), as the detection of exogenous fluorophore transcripts is usually low by short-read sequencing. Though one can use these tools for single-cell profiling studies, they are often inefficient and come with a high experimental cost due to the multiplex strategy.

### BRIEF DESCRIPTION OF THE DISCLOSURE

Among the various aspects of the present disclosure is the provision of Projection-TAGs and methods of use thereof.

In accordance with an aspect of the present disclosure, a Projection-TAG is provided. The Projection-TAG comprises an AAV plasmid, wherein the AAV plasmid comprises: a promoter; an RNA barcode; a fluorescent marker; and a regulatory element.

In some embodiments, the promoter comprises a chicken beta-actin (CAG) promoter; the RNA barcode is unique to a neuron projecting to a target region; the RNA barcode is 100 base pairs; the fluorescent marker comprises a fluorescent protein fused with a protein targeting a localization domain; the fluorescent marker comprises a GFP and oScarlet; the localization domain targets a nuclear membrane; the protein targeting the nuclear membrane is Sun1; and the regulatory element is a woodchuck hepatitis virus post-transcriptional regulatory element (WPRE).

In accordance with another aspect of the present disclosure, a method for multiplex tracing of a projection neuron in a brain of a subject is provided. The method comprises: administering a Projection-TAG to the subject, the Projection-TAG comprising an AAV plasmid, wherein the AAV plasmid comprises: a promoter; an RNA barcode; a fluorescent marker; and a regulatory element. The method further comprises: obtaining one or more biological samples from the subject; and applying an imaging modality to the one or more biological samples.

In some embodiment, the projection neuron comprises a neuron in a primary motor cortex (MOp) and a primary somatosensory cortex (SSp); the RNA barcode of the Projection-TAG is unique to the neuron projecting to a target region; the RNA barcode is comprised of 100 base pairs, the target region comprises an intratelencephalic (IT) target and an extratelencephalic (ET) target; the IT target comprises a contralateral MOp (cMOp) and a contralateral SSp (cSSp); the ET target comprises a ipsilateral ventral posterior nucleus of the thalamus (VP) region, a ipsilateral periductal grey (PAG) region, a ipsilateral medulla (MY) region, a lumbar spinal cord (SCL) region, and a sacral spinal cord (SCS) region; the imaging modality applied to the one or more biological samples is selected from immunofluorescent staining, fluorescence in situ hybridization (FISH), flow cytometry and fluorescence-activated cell sorting (FACS), single-cell RNA-sequencing (scRNA-seq), single-nucleus RNA-sequencing (snRNA-seq), and single-nucleus ATAC-sequencing (snATAC-seq); the fluorescent marker comprises a fluorescent protein fused with a protein targeting a localization domain; the fluorescent protein comprises GFP and oSCarlet; the fluorescent protein is photobleached.

Depending upon the embodiment, the promoter is selected from CAG, Ef1a, Syn, and promoters known to those of ordinary skill at the time of filing. Depending upon the embodiment, the fluorescent marker/reporter/protein is selected from GFP, oScarlet, YFP, RFP, and fluorescent markers/reporters/proteins known to those of ordinary skill at the time of filing.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a schematic of the experimental and analytical workflow of tagging neuronal projections of the primary motor area (MOp) and primary somatosensory area (SSp) using Project-TAGs. The workflow comprises cloning, viral packaging, multiplex projection tracing, tissue collection, TAG detection, and data acquisition.

FIG. 1B is a heatmap showing the enrichment of cells labeled with individual Projection-TAGs by multiplexed FISH in brain regions of injections. Cell counts are normalized by the max values in each brain region. Individual TAGs and their injection sites are shown as on the column.

FIG. 1C is a set of representative fluorescent images of MOp showing cells labeled with the fluorescent Sun1-GFP signal and fluorescent FISH signals from Projection-TAGs. Quantification shows the overlap between cells that are GFP+ and/or Projection-TAG+. FISH probes uniquely targeting Projection-TAGs (barcodes (BCs) 1-7) were visualized in separate imaging channels, shown as individual TAGs, and the signals were aggregated pos-hoc, shown as TAGs. Scale bar=100 μm.

FIG. 1D is a bar plot showing the percentage of nuclei expressing select marker genes (>0 UMIs for snRNA-seq, >cutoffs for target amplification) in individual snRNA-seq libraries prepared by FACS and target amplification. Bar and error bar indicate the average and standard deviation across libraries. (p=5.3e-06, F(2,10)=51.77, one-way ANOVA; **p=0.006 between Projection-TAGs and GFP in snRNA-seq, **p=0.003 for Projection-TAGs between snRNA-seq only and snRNA-seq with target amplification, ***p=0.001 between GFP in snRNA-seq and Projection-TAGs in snRNA-seq with target amplification, post-hoc t-tests with Bonferroni correction).

FIG. 1E is a box plot showing the false detection rate of Projection-TAGs due to ambient RNA contamination in snRNA-seq libraries. Dotted line shows the highest FDR due to ambient RNA contamination at 0.0026 among all libraries.

FIG. 1F is a spatial map of Projection-TAG+ cells in select cortex areas from a brain section (−0.65 mm relative to bregma). Cells are downsampled to 200 per Projection-TAG and colored by the injection site of the corresponding TAG. Grey dots are cells segmented based on DAPI signal.

FIG. 1G is a heatmap showing the distribution of projection neurons in select cortex areas from FIG. 1F. ACA: anterior cingulate area, MOs: secondary motor area, MOp: primary motor area, SSp: primary somatosensory area, SSs: supplemental somatosensory area, VISC: visceral area, AI: agranular insular area.

FIG. 2A is a uniform manifold approximation and projection (UMAP) visualization of single-nucleus RNA-seq (snRNA-seq) data showing 10,000 downsampled nuclei, colored by transcriptional subtypes.

FIG. 2B is a UMAP visualization of snATAC-seq data showing 10,000 downsampled nuclei, colored by transcriptional subtypes.

FIG. 2C is a UMAP visualization of snRNA-seq nuclei, colored by their projection targets. 10,000 downsampled Projection-TAG− nuclei are colored grey and displayed as the background. 500 nuclei (or all nuclei if total nuclei count of one projection is less than 500) expressing only the corresponding Projection-TAG are downsampled for each projection target and displayed.

FIG. 2D is a schematic showing the hierarchical clustering of 35 snRNA-seq clusters based on the average expression of top 100 marker genes (by FDR) per cluster, and their annotations of class, cell type, and subtype.

FIG. 2E is a heatmap showing the average expression of select marker genes in individual snRNA-seq clusters.

FIG. 2F is a plot of average accessibility of canonical cell type-specific marker genes in snATAC-seq nuclei, grouped by transcriptional clusters. The chromatin accessibility is displayed as the average frequency of sequenced DNA fragments per nucleus for each cluster, grouped by 50 bins per displayed genomic region.

FIG. 2G is a dot plot showing fraction of snRNA-seq nuclei, positive for Projection-TAG of each projection, from each transcriptional cluster.

FIG. 2H is a set of representative FISH images showing the layer distribution of projection neurons in MOp from a brain section (−0.91 mm relative to bregma). Colors denote the projection targets based on the expression of Projection-TAGs. Scale bar=150 μm. Top: composite image of all FISH channels, bottom left and right: images of a subset of Projection-TAGs injected into IT targets and ET targets, respectively.

FIG. 2I is a set of graphs of FISH quantification showing the fraction of MOp cells retrogradely labeled by each Projection-TAG in each layer. Bar and error bar indicate the average and standard deviation across six slices from two mice.

FIG. 2J is a set of RNA-UMAP showing subclustering of L5 PT neurons labeled with each Projection-TAG.

FIG. 2K is a heatmap showing the z-score of average expression of genes (column) differentially expressed in L5-PT neurons positive for each Projection-TAG (row).

FIG. 3A is a bar graph plot showing the percentage of snRNA-seq nuclei, grouped by the number of unique Projection-TAGs detected in each nucleus.

FIG. 3B is a heatmap demonstrating the pairwise overlapping of snRNA-seq nuclei positive for any two Projection-TAGs, shown as the percentage of nuclei positive for target 1 TAG (column) that are also positive for target 2 TAG (row).

FIG. 3C is a heatmap showing the Projection-TAG (row) detected in individual snRNA-seq nuclei (column) expressing only one Projection-TAG. Nuclei are ordered based on the projection patterns, and only groups that passed the FDR cutoff (at least 60 nuclei) were shown on the heatmaps.

FIG. 3D is a heatmap showing the Projection-TAG (row) detected in individual snRNA-seq nuclei (column) expressing multiple Projection-TAGs. Nuclei are ordered based on the projection patterns, and only groups that passed the FDR cutoff (at least 60 nuclei) were shown on the heatmaps.

FIG. 3E is a Venn diagram showing the overlap between VP-TAG-expressing and other ET-TAG-expressing snRNA-seq nuclei. Other ET-TAGs include Projection-TAGs injected into PAG, MY, SC_{L}, and SC_{S}.

FIG. 3F is a representative FISH image showing MOp cells labeled with VP- and other ET-TAGs. Scale bar=100 μm.

FIG. 3G is a graph showing the distribution of transcriptional subtypes in each projection group. Transcriptional subtypes with <60 nuclei from each projection feature group were excluded and cell types that make up at least 10% nuclei in the projection group are annotated on the plot.

FIG. 3H is a heatmap showing the z-score of average expression of 118 genes (row) differentially expressed in transcriptional L5 PT nuclei in each projection group (column).

FIG. 4A is a pie chart categorizing snATAC-seq peaks based on their genomic locations. Peaks are mapped to one of the three categories: promoter regions (−1,000 bp to +100 bp of transcription start site [TSS]), distal regions (<200 kb upstream or downstream of TSS or within gene body, excluding promoter), and intergenic regions (>200 kb upstream or downstream of TSS, excluding gene body).

FIG. 4B is a schematic showing overlapping cell type-specific peaks and projection-specific peaks in snATAC-seq data.

FIG. 4C is an analytical workflow of identifying putative enhancers and silencers and their regulated genes.

FIG. 4D is a set of heatmaps showing 18,088 pairs of cell type-specific pu.Enhancers and regulated genes. Left heatmaps show the average accessibility of pu.Enhancer peaks (row) in individual cell types (column) in snATAC-seq data and right heatmaps show the average expression of regulated genes (row) in individual cell types (column) in snRNA-seq data. On the right side next to the heatmaps, the color bar indicates the cell types in which the peak and gene have highest accessibility and expression, and the bar plot shows the Pearson correlation coefficient (R) between the accessibility of the peak and the expression of the gene in individual cell types.

FIG. 4E is a set of heatmaps showing 2,739 pairs of cell type-specific pu.Silencers and regulated genes. Left heatmaps show the average accessibility of pu.Enhancer peaks (row) in individual cell types (column) in snATAC-seq data and right heatmaps show the average expression of regulated genes (row) in individual cell types (column) in snRNA-seq data. On the right side next to the heatmaps, the color bar indicates the cell types in which the peak and gene have highest accessibility and expression, and the bar plot shows the Pearson correlation coefficient (R) between the accessibility of the peak and the expression of the gene in individual cell types.

FIG. 4F is a set of heatmaps showing 3,545 pairs of projection-specific puEnhancers and regulated genes. Left heatmaps show the average accessibility of pu.Enhancer peaks (row) in nuclei positive for each Projection-TAG (column) in snATAC-seq data and right heatmaps show the average expression of regulated genes (row) in nuclei positive for each Projection-TAG (column) in snRNA-seq data. On the right side next to the heatmaps, the color bar indicates the projection targets in which the peak and gene have highest accessibility and expression, and the bar plot shows the Pearson correlation coefficient (R) between the accessibility of the peak and the expression of the gene in nuclei positive for each Projection-TAG.

FIG. 4G is a set of heatmaps showing 4,200 pairs of projection-specific pu.Silencers and regulated genes. Left heatmaps show the average accessibility of pu.Enhancer peaks (row) in nuclei positive for each Projection-TAG (column) in snATAC-seq data and right heatmaps show the average expression of regulated genes (row) in nuclei positive for each Projection-TAG (column) in snRNA-seq data. On the right side next to the heatmaps, the color bar indicates the projection targets in which the peak and gene have highest accessibility and expression, and the bar plot shows the Pearson correlation coefficient (R) between the accessibility of the peak and the expression of the gene in nuclei positive for each Projection-TAG.

FIG. 4H is a set of plots showing chromatin accessibility of chr19-39185083-39185986. (Top) Scatter plot showing the correlation between the average accessibility of peak chr19-39,185,083-39,185,986 in snATAC-seq and the average expression of gene Htr7 in snRNA-seq in individual cell types (chromatin accessibility and gene expression are normalized to their max values). The dotted line represents the line of best fit. (Bottom left) Chromatin accessibility at the genomic locus of chr19-39,185,083-39,185,986, displayed as the average fraction of transposase-sensitive fragments per nucleus in each cell type (grouped by 50 bins per displayed genomic region). Accessibility at each locus (y axis) is scaled to the max value across all cell types. (Bottom right) Expression of Htr7 in each cell type.

FIG. 4I is a set of plots showing chromatin accessibility of chr13-82081120-82082049. (Top) Scatter plot showing the correlation between the average accessibility of peak chr13-82,081,120-82,082,049 and the average expression of gene Polr3g in nuclei positive for each Projection-TAG (chromatin accessibility and gene expression are normalized to their max values). (Bottom left) Chromatin accessibility at the genomic locus of chr13-82,081,120-82,082,049, displayed as the average fraction of transposase-sensitive fragments per nucleus in nuclei positive ach Projection-TAG (grouped by 50 bins per displayed genomic region). (Bottom right) Expression of Polr3g in nuclei positive for each Projection-TAG. Colors indicate the injection site of the corresponding TAG. Nuclei positive for SC_{L}- and SC_{S}-TAGs are combined for visualization due to low cell number.

FIG. 5A is a set of violin plots showing IEG scores in snRNA-seq nuclei for each projection between treatments (***p=4.6e-14 for cMOp, **p=0.004 for cSSp, p=1 for VP, p=0.99 for PAG, p=0.99 for MY, p=0.4 for SC_{L}, p=0.2 for SC_{S}, one-side t-test with Bonferroni correction).

FIG. 5B is a set of volcano plots showing significantly induced IEGs (log 2FC >0.5) in activated nuclei from CYP-treated mice compared to the same number of randomly sampled nuclei (with matched transcriptional subtypes) from saline-treated mice for the two IT projections.

FIG. 5C is a set of representative FISH images showing the expression of Homer1 and Projection-TAGs in MOp (from brain section approximately −0.2 mm relative to bregma) from saline- and CYP-treated mice. Scale bar: 100 μm.

FIG. 5D is a graph showing the quantification of Homer1+ cells in MOp and SSp areas from CYP- and saline-treated mice (0 to −0.92 mm relative to bregma, nine slices from three mice per group, ***p=1.9e-5, one-side t-test).

FIG. 5E is a graph showing the percentage of Homer1+ MOp and SSp cells in each projection. Fold change is calculated as the percentage in individual CYP slices, divided by the average percentage across saline slices. Bar and error bar indicate the mean and standard deviations across nine slices per group.

FIG. 6A is a summary of available high-throughput neuroanatomical tools.

FIG. 6B is a set of representative fluorescent images of the localization of fusion proteins in different cellular compartments in fixed HEK cells. Scale bar: 10 μm.

FIG. 6C is a set of representative fluorescent images of the fluorescent labeling using fusion proteins of nuclear resuspension extracted from HEK cells. Scale bar: 20 μm.

FIG. 6D is a summary of the fluorescent proteins and their ability of labeling of cells and nuclei of HE K cells.

FIG. 6E is a set of scatter plots showing the gate of sorting labeled HEK nuclei (DAPI+ and GFP+) extracted from HEK cells expressing Sun1-GFP.

FIG. 7A is a set of plots showing (left) pairwise Euclidean distance between any two of the 50 BCs and (right) a box plot showing the average Euclidean distance of individual BCs to other BCs.

FIG. 7B is a heatmap showing counts of sequencing reads that map to individual BCs (row) in individual HEK samples (column) in RNA-seq. Each HEK sample was either transfected with a plasmid expressing a unique Project-TAG BC or no transfection.

FIG. 7C is a set of representative FISH images and corresponding showing the detection of individual Projection-TAGs (BCs 1-12) in individual HEK samples each expressing a unique TAG or the HEK sample with no transfection. The bar plots show the average signal intensity from each Projection-TAG channel across a field of view with 1 mm by 1 mm. The average intensity is normalized by the max values of each sample and each channel. In HEK samples transfected with Projection-TAG plasmids, the dotted line and the associated number represent the false detection rate (average intensity of Projection-TAGs not expressed in a given sample), whereas the number on the right indicates the true detection rate (intensity from the Projection-TAG expressed in a given sample). In the HEK sample with no transfection, the dotted line and the associated number represent the background (average intensity of all Projection-TAGs). Scale bar: 20 μm.

FIG. 7D is bar plot showing the average detection rate of Projection-TAGs in individual samples, error bars are standard deviation. True detection is the detection of the Projection-TAG expressed in a given sample transfected with one Projection-TAG plasmid, false detection is the detection of Projection-TAGs not expressed in the same samples, and the background is the detection of individual Projection-TAGs in the HEK sample with no transfection.

FIG. 7E is a bar plot showing average detection rate of Projection-TAGs across all samples, error bars are standard deviation.

FIG. 7F is a set of representative images showing that photobleaching process removed the native Sun1-GFP fluorescence from HEK cells. Scale bar: 50 μm.

FIG. 7G is a set of representative images showing that DNase incubation removed the fluorescent signal of Alexa-488 conjugated with GAPDH FISH probe in HEK cells. Scale bar: 50 μm.

FIG. 7H is a set of representative images showing that photobleaching process significantly reduced the native Sun1-GFP fluorescence from mouse brain sections. Scale bar: 100 μm.

FIG. 7I is a set of representative images showing that DNase incubation removed the fluorescent signals of Alexa-488 conjugated with the FISH probe targeting GFP in mouse brain sections. Scale bar: 100 μm.

FIG. 8A is a set of box plots showing the time kinetics of Projection-TAG expression in the cortex. Animals received MY or SC_{S} injection with the Projection-TAG AAV (expressing TAG 2), cortical samples were collected at different time points post injection, and the Projection-TAG expression is measured using qPCR (n=4 for each time point and each injection). ΔCT is calculated as the CT value of TAG 2 minus that of GAPDH for each sample, ΔΔCT is calculated as the ΔCT at each time point minus the ΔCT at 4 weeks post-injection.

FIG. 8B is an RNA-UMAP of 10,000 nuclei (1,000 downsampled Projection-TAG+ nuclei from each titer group, colored by titer, and 8,000 downsampled BC-nuclei, colored grey).

FIG. 8C is a set of schematics and corresponding volcano plots of DE genes in response to viral infection. DE analysis was performed comparing the expression of genes annotated in the Gene Ontology list GO:0009615 (response to virus) between Projection-TAG+ nuclei and Projection-TAG− nuclei (downsampled to match the Cell type distribution and nuclei counts to the Projection-TAG+ nuclei) in each tier group. Top panels show the number of upregulated (avg_log 2FC >0) and downregulated (avg_log 2FC <0) viral responding genes among the viral responding genes expressed (average expression >0.5) in snRNA-seq nuclei from each titer group. Fisher's exact test was performed to test if viral responding genes are significantly enriched among DE genes (p=0.81 and 0.77 for undiluted and diluted group, respectively). Bottom panels show the individual DE viral responding genes.

FIG. 8D is a set of schematics and corresponding representative FISH images of the medial part of the posterior parietal association area of the cortex (PTLp) visualizing Projection-TAGs injected into VP and PAG in a mouse receiving only VP injection (first row), a mouse receiving only PAG injection (second row), a mouse receiving VP and PAG injections at the same time (third row), and a mouse receiving PAG injection two weeks after VP injection (bottom row). Scale bar: 100 μm.

FIG. 8E is a graph quantifying VP-TAG+ cells in medial PTLp from different injection groups (nine slices from three mice each group). p=0.26, F(2,25)=1.72, one-way ANOVA.

FIG. 8F is a graph quantifying PAG-TAG+ cells in medial PTLp from different injection groups (9 slices from three mice each group). p=0.27, F(2,25)=1.4, one-way ANOVA.

FIG. 8G is a set of violin plots showing the VP-TAG UMIs from snRNA-seq nuclei expressing only VP-TAG and co-expressing VP/PAG-TAGs in three snRNA-seq libraries that contain at least 60 nuclei co-expressing VP/PAG-TAGs. p=0.99 (RNA-1), 0.01 (Multiome-3), and 0.06 (Multiome-3), student t-tests with Bonferroni correction.

FIG. 8H is a set of violin plots showing the PAG-TAG UMIs from snRNA-seq nuclei expressing only PAG-TAG and co-expressing VP/PAG-TAGs in the snRNA-seq libraries shown in g. p=0.5 (RNA-1), 0.1 (Multiome-3), and 0.6 (Multiome-3), student t-tests with Bonferroni correction.

FIG. 8I is a set of representative images showing the co-localization of GFP fluorescent signals with VP-TAG signal from FISH. Scale bar: 100 μm.

FIG. 8J is a set of representative images showing the co-localization of GFP fluorescent signals with PAG-TAG signal from FISH. Scale bar: 100 μm.

FIG. 9A is a chart showing key sequencing and Projection-TAG detection metrics from snRNA-seq libraries prepared using different commercial kits from nuclei of the same biological samples (n=2 for each kit).

FIG. 9B is a dot plot showing the effect of sequencing depth/saturation on the percentage of Projection-TAG+ nuclei in the snRNA-seq library that reached 500 k reads/nucleus. Sequencing reads were randomly downsampled to match the sequencing reads/nucleus metric shown on the plot.

FIG. 9C is a bar graph showing the effect of sequencing Read 2 length on the percentage of Projection-TAG+ nuclei in the snRNA-seq library shown in b. Sequencing reads were trimmed using “r2-length” parameter in cell ranger.

FIG. 9D is a set of plots showing detection of each Projection-TAG (left) and any Projection-TAGs (right) in snRNA-seq alone (x-axis) and in snRNA-seq library with target amplification (y-axis) in individual sequencing libraries. Blue line on each plot indicates the best fitted line.

FIG. 9E is a dot plot plot showing the target amplification performance as fold change of Projection-TAG+ nuclei between snRNA-seq library with target amplification and snRNA-seq alone in the snRNA-seq library shown in b. Sequencing reads/nucleus metric is labeled on the plot.

FIG. 9F is a heatmap Heatmap showing the percentage of Projection-TAG+ nuclei expressing each Projection-TAG (labeled as the injection site of the corresponding Projection-TAG on the row) from each animal.

FIG. 9G is a set of box plots showing the Projection-TAG UMI count from snRNA-seq nuclei expressing each Projection-TAG (labeled as the injection site of the corresponding Projection-TAG on the row).

FIG. 9H is a set of violin plots showing the Projection-TAG UMI count from snRNA-seq nuclei expressing the corresponding Projection-TAG from two animals (x-axis), in which different Projection-TAGs are injected into the same brain region for the regions shown on the plot. p=0.08 cMOp, p=0.12 VP, p=0.65 PAG, ***p=1.5e-4 MY.

FIG. 10A is a set of schematics showing the stereotaxic injection of individual Projection-TAGs into each of the projection targets of the cortex.

FIG. 10B is a set o representative images showing the native fluorescence of Sun1-GFP in the injection sites. Scale bar: 1 mm.

FIG. 10C is a set of representative FISH images (magnified view of the region highlighted in FIG. 10B) showing the expression of Projection-TAGs in the injection sites. Multiplexed FISH was performed to visualize the Projection-TAGs (TAGs 1-7) in the projection targets of the cortex (highlighted with dotted lines). Scale bar: 300 μm.

FIG. 11A is a workflow of multiplexed FISH experiment and imaging analysis.

FIG. 11B is a set of representative FISH images (top row) showing the Projection-TAGs labeled projection neurons to each target in the MOp. The bottom row shows cell segmentation of the projection neurons from the images on the left panels using machine-learning-based algorithms. Colors denote individual objects identified by the algorithms.

FIG. 11C is a set of graphs of rostro-caudal caudal distribution of neurons projecting to each target in ipsilateral cortex areas. Smooth lines were fitted to the quantification of 22 brain sections from two mice, and the grey ribbons show the standard error of the mean (SEM).

FIG. 11D is a set of representative images and corresponding Venn diagrams. The images show VP- and MY-projecting neurons labeled with Projection-TAGs in the select cortex areas from three brain sections (top: 1.26 mm, middle: 0.23 mm, bottom −0.95 mm relative to bregma). Venn diagrams show overlapping of cells projecting to both targets (p=7.57e-14, 1.23e-44, 3.30e-41 for top, middle, and bottom diagrams, respectively. Hypergeometric tests). Scale bar: 200 μm.

FIG. 11E is a heatmap showing the cell counts of neurons projecting to each target in each select cortex area.

FIG. 12A is a workflow of the data analysis of the snRNA-seq and snATAC-seq and cell type and projection annotation.

FIG. 12B is a set of graphs showing snRNA-seq library metrics. Top row displays number of nuclei passed quality control. Middle row displays box plots of number of genes per nucleus (log 10 transformed) and the bottom row displays the number of UMIs per nucleus. Boxes indicate quartiles and whiskers are 1.5-times the interquartile range (Q1-Q3). The median is a white line inside each box. The distribution is aggregated across all samples and displayed on the horizontal histogram.

FIG. 12C is a set of RNA-UMAP s of 1,000 downsampled nuclei per group. Nuclei were colored by sequencing kits (left) or the origin of brain regions (right).

FIG. 12D is a is a graph showing the composition of transcriptional cell types in each snRNA-seq library.

FIG. 12E is a dot plot showing the marker genes used to annotate subtypes of non-neuronal cells.

FIG. 12F is a dot plot showing the marker genes used to annotate subtypes of neuronal cells.

FIG. 12G is a heatmap showing the percentage of snRNA-seq nuclei in each cluster (row) assigned by anchoring analysis to the cell types (column) of a published scRNA-seq dataset of mouse MOp and SSp.

FIG. 13A is a set of graphs showing snRNA-seq library metrics. Top row displays number of nuclei passed quality control. Middle row displays box plots of number of peaks per nucleus (log 10 transformed) and the bottom row displays the number of fragments that overlap with peaks per nucleus. Boxes indicate quartiles and whiskers are 1.5-times the interquartile range (Q1-Q3). The median is a white line inside each box. The distribution is aggregated across all samples and displayed on the horizontal histogram.

FIG. 13B is a set of graphs showing the distribution of snATAC-seq fragment lengths in nuclei grouped by transcriptional subtypes.

FIG. 13C is a heatmap showing correspondence of ATAC clusters (row) and transcriptional subtypes (column) of nuclei profiled combinatorially by snRNA-seq and snATAC-seq. Plot displays the percentage of nuclei within each ATAC cluster that is assigned to each transcriptional subtype.

FIG. 13D is a box plot showing the percentage of Projection-TAG+ nuclei in each snRNA-seq library in each subtype.

FIG. 13E is a heatmap showing the significance levels of the Bonferroni-corrected p values of pairwise hypergeometric tests of subtype distribution, shown in FIG. 2g, between any of the two projections. Hierarchical clustering was performed based on the subtype distribution for each projection.

FIG. 13F is a dot plot showing the expression of Hpgd and Slco2a1 in individual projections.

FIG. 14A is a heatmap showing the Bonferroni-corrected p values from pairwise hypergeometric tests of overlapping between any of the two projections. Two projections were considered highly significantly overlapping with p<1e-200 (shown as red to purple on the heatmap).

FIG. 14B is a set of representative FISH images showing projection neurons labeled by Projection-TAGs in the coronal section showing motor cortex from different anterior-posterior sections (top) and zoomed-in images (bottom) of the highlighted region demonstrate expression of two unique Projection-TAGs injected into downstream cMOp and cSSp; middle: demonstrate expression of three unique Projection-TAGs injected into the VP, PAG, and MY; right: demonstrate expression of two unique Projection-TAGs injected into SC_{L} and SC_{S}). Scale bar: 1 mm (top), 100 μm (bottom).

FIG. 14C is a set of images and summary diagram showing tracing of brain-wide axon collaterals of MOp neurons and of the anatomical hierarchy of axonal projections from MOp to the seven target regions.

FIG. 14D is a is a set of images and summary diagram showing tracing of brain-wide axon collaterals of SSp neurons and the anatomical hierarchy of axonal projections from SSp to the seven target regions.

FIG. 14E is a heatmap showing the number of DE genes (FDR<0.05) between any two projections.

FIG. 14F is a heatmap showing the number of DA peaks (FDR<0.05) between any two projections.

FIG. 15A is a graph showing the distribution of projection feature to VP and/or other ET targets (defined in FIG. 3h-j) in Projection-TAG+ nuclei in each subtype.

FIG. 15B is a schematic of a single neuron (MouseLight AA0923) whose cell body reside in the MOp L5 and axonal end points terminating in thalamus, PAG, MY, an axon follow through the corticospinal tract. Screenshots were acquired from the Janelia MouseLight data portal.

FIG. 16A is a box plot showing the overlapping of putative GREs identified from this study to the putative GREs identified by studies from the ENCODE consortium. Overlap is significantly higher with studies of the brain compared to those of non-brain organs (***p=8.26e-05, t-test). Brain studies: ENCSR422CGZ, ENCSR694ZBB, ENCFF426KUB, and ENCSR410XMU. Studies of organs that are not the nervous system: ENCSR052AZT (stomach), ENCSR100TMZ (lung), ENCSR296UQL (liver), and ENCSR18001F (limb).

FIG. 16B is a heatmap showing the chromatin accessibility of two cell type-specific pu.Enhancers in individual Transcriptional subtypes. The peaks of the two pu.Enhancers overlap with the genomic coordinates liftover from two functional enhancers previously identified based on human snATAC-seq data. Labels in the grey boxes show the functional enhancers and the cell types in which the enhancers exhibited highest activity in experimental validation from the previous study.

FIG. 16C is a set of plots showing the accessibility of chr19-10784771-10785580. Top: Scatter plot showing the correlation between the accessibility of peak chr19-10,784,771-10,785,580 and the expression of gene Fth1 in individual transcriptional subtypes (chromatin accessibility and gene expression are normalized to their max values). The dotted line represents the line of best fit. bottom left: chromatin accessibility at the genomic locus of chr19-10,784,771-10,785,580, displayed as the average fraction of transposase-sensitive fragments per nucleus in each subtype (grouped by 50 bins per displayed genomic region). Accessibility at each locus (y axis) is scaled to the max value across all cell types. Bottom right: expression of Fth1 in each cell type. Colors indicate Transcriptional subtypes.

FIG. 16D is a set of plots showing the accessibility of chr18-39362615-39363500. Top: Scatter plot showing the correlation between the accessibility of peak chr18-39,363,615-39,363,500 and the expression of the gene Kctd16 in nuclei positive for individual TAGs (chromatin accessibility and gene expression are normalized to their max values). bottom left: chromatin accessibility at the genomic locus of chr18-39,363,615-39,363,500, displayed as the average fraction of transposase-sensitive fragments per nucleus in each projection (grouped by 50 bins per displayed genomic region). Bottom right: expression of Kctd16 in each projection. Colors indicate nuclei positive for individual TAGs. Nuclei positive for SC_{L}- and SC_{S}-TAGs are combined for visualization due to low cell number.

FIG. 17A is a graph showing the cumulative scores of quantified behaviors between mice treated with saline and CYP. CYP treatment resulted in significant increases in the spontaneous behaviors like abdominal licking and squashing behaviors compared to saline treatment. Ribbons show SEM. ** p=0.0034, one-way ANOVA. p=NaN (0 min), NaN (5 min), NaN (10 min), NaN (15 min), 0.25 (20 min), 0.07 (25 min), *0.05 (30 min), *0.04 (35 min), *0.04 (40 min), **0.001 (45 min), **0.006 (50 min), ***0.0005 (55 min), ***0.0003 (60 min), respectively, post-hoc one-sided t-test, n=5-6 mice/group.

FIG. 17B is an RNA-UMAP (left) and ATAC-UMAP (right) of 5,000 downsampled nuclei from each treatment group, colored by treatments.

FIG. 17C is a violin plot showing IEG scores of all snRNA-seq nuclei from CYP and saline. p=0.16.

FIG. 17D is a set of violin plots showing IEG score of nuclei between treatments from CYP-activated transcriptional subtypes. ***p=2.6e-7 Pvalb, ***p=6.2e-7 L5 IT_Pcdh15, ***p=1.3e-6 L6 IT_Galnt14, ***p=1.5e-6 L6 IT_Fst, ***p=4.6e-5 Vip, ***p=1.5e-4 L4 IT_Mamdc2, ***p=3.8e-4 L6 CT_Ephb1, ***p=3.8e-4 L5/6 NP, **p=1.3e-3 L6 CT_Zfpm2, **p=4.6e-3 L2/3 IT_Schip1, *p=0.02 L5 IT_Deptor, *p=0.04 L4 IT_Tafa2.

FIG. 17E is a set of representative FISH images showing Fos expression in select cortex areas (top: 1.33 mm, middle: −0.95 mm, bottom −1.67 mm relative to bregma). Scale bar: 400 μm.

FIG. 17F is a set of bar plots plot showing the percentage of Homer1+ cells in each projection group from FISH analysis. Nine slices from three mice per group, ***p=6.7e-8 cMOp, ***p=2.9e-6 cSSp, *p=0.02 VP, ***p=1.2e-5 PAG, ***p=1.9e-4 MY, **p=0.002 SC_{L}, **p=0.006 SC_{S}.

### DETAILED DESCRIPTION OF THE DISCLOSURE

The present disclosure is based, at least in part, on the discovery that the novel Projection-TAG technology enables comprehensive mapping of projection neurons including multi-modal profiling and multiplex projection tracing in the brain.

As disclosed herein, Projection-TAGs introduce a retrograde AAV platform that allows multiplex tagging of projection neurons using RNA barcodes. By using Projection-TAGs, multiplex projection tracing of the mouse cortex and high-throughput single-cell profiling of the transcriptional and epigenetic landscapes of the cortical projection neurons were performed. Projection-TAGs can be leveraged to obtain a snapshot of activity-dependent recruitment of distinct projection neurons and their molecular features in the context of a specific stimulus. Given its flexibility, usability, and compatibility, Projection-TAGs can be readily applied to build a comprehensive multi-modal map of brain neuronal cell types and their projections.

Further, the Projection-TAGs retrograde AAV platform described herein allows multiplex tracing of projection neurons by tagging each neuronal projection with a unique RNA barcode. The key component of Projection-TAGs is a set of engineered retrograde AAVs each expressing a unique barcode, which acts as the projection identifier. With this scheme, neurons projecting to a target region are uniquely tagged by a retrograde AAV-mediated RNA barcode and multiplex projection tracing can be achieved by injecting a unique barcode-expressing Projection-TAG AAVs into each of the downstream target regions predefined for investigation. To map projection targets, RNA barcodes are demultiplexed using commercial assays, allowing multiplex neuroanatomical tracing studies and high-throughput multi-modal profiling of projection neurons in single animals.

As demonstrated herein, Projection-TAGs offer a powerful, high-throughput platform to perform systemic multiomic analyses to gain insight into the spatial location, gene expression, and chromatin accessibility profiles of diverse projection neurons. Lastly, data shows that Projection-TAGs can be leveraged to obtain a snapshot of activity-dependent recruitment of distinct projection neurons and their molecular features in the context of a stimulus of interest by combining Projection-TAG s with Act-seq. The number of projections that can be labeled with Projection-TAG AAVs is not inherently constrained. Projection-TAG s can be easily scalable, including 50 screened BCs described herein that may be packaged to increase multiplexing of projection tagging. Projection-TAGs are readily deployable and will democratize the study of the nervous system in neuroscience labs without any specialized equipment.

Projection-TAG RNA barcodes may comprise the sequence found in Table 1.

TABLE 1
Projection-TAG RNA barcodes

| Barcode ID | Sequence |
|---|---|
| 1 | ACCTGTAGAACAACCAGGTGATGTTCCACGTGGACATCTAGCAGGTGCTGATCGAGAAG |
| | GACTACTTGAACGACATGCTCTAGCTCTAGATCTACGTCTT (SEQ ID NO: 1) |
| 2 | TGAACCACTTCTAGGTGGACTTGGTCTAGATCATGTAGTACTTCTAGCTGTTGGTGATC |
| | GACCTCAAGTTCAACAACTTGATCCTCTTGGTCATGGTGGA (SEQ ID NO: 2) |
| 3 | TCGACAAGCTCCACTAGCAGGTCATCATGTTGCACCAGCACCACAACTTCATGTAGTTG |
| | TAGGTGTAGGTCATCGTCATCTAGTACCAGGACCAGAAGAT (SEQ ID NO: 3) |
| 4 | TGGACCAGTAGTTCGTCCTGATCTTGCAGTTCTTGTTGTTGAACCAGCTGTTCTAGCTC |
| | GTCATGCAGATGGACGAGTTCTAGGACTACTTCGTCTTCAT (SEQ ID NO: 4) |
| 5 | AGGTGTTGATGTACATCCTCATGGAGTTCTTGGTCCAGCTCAACGTGTTCAACCTCGAG |
| | TACCAGAACTTCGAGGTGTACGAGAACGTCGTGTTGTTCAT (SEQ ID NO: 5) |
| 6 | ACGTCAACGTGGACGTCCTCGTGCAGGTCGTGTAGGTGGTGTTGGTCAACCAGCAGAAC |
| | TACCAGGAGCTGGAGCTCCAGTTGCACTTCAAGGAGTAGCA (SEQ ID NO: 6) |
| 7 | ACTTGGAGGTGTTGCAGGTGGTGCACAAGTTCCTCCACAAGATGTACTTGGAGAACCAG |
| | CTCGACTTGGTCTTGGACAACGTCGTGTTCTAGCAGGTCCA (SEQ ID NO: 7) |
| 8 | AGGTGTTCCTCAAGGTGGAGATGTACATGTAGGTCATCCAGGACTTCTTCATCCTGTTG |
| | TTGATCGAGTAGTTCATGGTGTACCTGTTCCAGTTGTACGT (SEQ ID NO: 8) |
| 9 | ACAAGGAGTTCATGCAGCTCTTCAAGATCCAGTAGGAGTTCTTGCACATCAAGCTCGTC |
| | TTCATCAACATGTTGATCTTGATGAAGCAGTTCTACAACCT (SEQ ID NO: 9) |
| 10 | CGATGCTGTACGAGGTGGACTTGATGATGATGCACATGGTGCTCCTCCTCGAGCTCTTG |
| | TACGTGGACTTGGAGCTGTAAATCTAGGTGAAGCTGAAGCT (SEQ ID NO: 10) |
| 11 | AGAACTTGCACCACGACATCGAGCTCCAGGTCGTCGTCATCATCGTCATGGAGGACTTG |
| | GTCGACGACGTCTAGTAGGTGGACGAGAAGAAGCAGTTGGT (SEQ ID NO: 11) |
| 12 | TCATCTTCATGCTCTAGGTCATGTAGAAGGACCAGTTCCTCTTGCTGTAGATCAAGGAC |
| | TTCGTCAACCTCGTGATGCTCATGTTGCACCTGGACGTGAA (SEQ ID NO: 12) |
| 13 | AGGTCAAGAAGTACATCATGCTGTACGAGGTGAAGATCAAGGTGTACATGTTCTACCAG |
| | TTGCTCTTCGTGATGGAGAAGCTGTAGCTGTTGCTGGTCTA (SEQ ID NO: 13) |
| 14 | TGTACTACATCTACTAGTACTACGTCTAGGTCCTGGTGTAGGAGCTCTTCGTGGAGTTC |
| | GTCTAGTAGATGCACGTGTAGTACAAGCAGAACCTCGAGTA (SEQ ID NO: 14) |
| 15 | AGGAGCTGATGGAGTTCTTCATCTTGATCGTCAACGTCTACGTCTAGGTGTACGAGAAC |
| | TAGTAGATCCTCTTGTACGAGAACAACGTGGAGCTCATGCA (SEQ ID NO: 15) |
| 16 | TGTACTAGCTGAAGTTGATCAAGTTGCTGATCCTGGTCTAGGTGGTCTACTTCATCTAG |
| | TAGGACTTCTACTTCGAGGAGATCATCGACAAGCACCACTT (SEQ ID NO: 16) |
| 17 | TCCTCGTGGAGGTCGTGTTGTTGTACTAGTTGCAGTTGTACGAGGAGATGCACGAGTTC |
| | GACATGGAGATGATGGTGAACAACGTCGAGTTCATGATCTA (SEQ ID NO: 17) |
| 18 | AGGTCGTCAAGATCGACCTGAAGTAGTAGTTCTAGATGCTGTTGATCCTCAAGTAGTAC |
| | CAGAACTACTTGTTGTACGTCGTGCTGGTGGACTACGAGGA (SEQ ID NO: 18) |
| 19 | AGTTGTTGCAGGACCAGTTGCAGGTCCTGCTGGTGCTCTAGGTCATCGAGGACAAGCAG |
| | CACGTGGACATGGACTTGGAGATGATGCTGCTCATGTACTT (SEQ ID NO: 19) |
| 20 | TGTTGATGCAGTAGATGGTCGTGTTGTTGTAGATGGTGTTGGACGTGCTGTTGAACCAC |
| | CTGTTCTAGTTGAAGTTGATGCACGACCACAACATCAAGCA (SEQ ID NO: 20) |
| 21 | TGTTCTTCGTGATGGTGTTGGTCGTGCACATCCACTAGTTCGTGTAGCTGCTGATGGTG |
| | GAGGTCTACATCCTCAAGGACGACAAGCTGCAGTACGTCGT (SEQ ID NO: 21) |
| 22 | TGTTCGTGCTGGTGTTCGACCTCAACAAGTTGTTCGTGAAGGTGCTGTTGATCCACCAC |
| | ATGCTGCTGCTCTAGGTCATGATGGTGAAGGACTAGTAGTA (SEQ ID NO: 22) |
| 23 | TCGAGGTGTTCGAGTTCGTGGTGGTGTTGAAGCTGTTCTACATCATGAACAACGTCTTC |
| | CTGTTGTAGAAGGAGGTCTTGGACAAGGTCCTGCAGGAGCA (SEQ ID NO: 23) |
| 24 | ACGAGGAGATGCTCAAGCTGGTGTACGTGTTGATGTTGTAGGACATGTAGTAGGAGGTG |
| | TTCCAGATGCTGCAGCTGGTGGTGTTGCTCGTGGTCTTGCA (SEQ ID NO: 24) |
| 25 | ACGTCCACATGTTGGAGATGTTGTTGGACAAGTACGAGCAGCACTTGTTCTTGGACGAC |
| | TTCCACATCCTGATGATCCTCATGCTGATGATGGTGTACCA (SEQ ID NO: 25) |
| 26 | TCCTCAAGCAGATGTTCTTCTTGGTGCAGTTGTTGTTCTTCAAGTTCATGAAGGTGGTG |
| | AAGTTGCTGCAGTACTTCGTCTTCGTCGTGCTGTAGTAGGT (SEQ ID NO: 26) |
| 27 | TGTTCTTCATGTACAAGCTGGAGGTCGTGCAGATGGAGATGCACGAGATCTACCTGGTG |
| | TAGCTGAAGCTCGTGATCAAGGTGGTGATCCACATCCTCGT (SEQ ID NO: 27) |
| 28 | TGATGGTCGTGATCTTGTTCGAGTTCATCCTGTAGATGGTGTACAAGTTGCTGCTCTAC |
| | TACGACATCCTCCTGTTCCACGTGTTGCTGTACTAGGAGTA (SEQ ID NO: 28) |
| 29 | TGATCGACGTGCTGCAGCTGGAGTTGAAGTAGTTCGTCGTCTAGTTCTTCATGTTGTTG |
| | CTCAAGTACGACCTGGTGGTCGTGGTCATCTACCTCTTCTT (SEQ ID NO: 29) |
| 30 | TGTAGTTCATGTTGTTGATCGTGCACGAGAAGGTGGTCCTGGTCTTATTGGTCTTGTTC |
| | AACGTCCTCATGCACCAGATCATCAACGAGTTGATCCTGGT (SEQ ID NO: 30) |
| 31 | TGTACCACATGTTCTAGATGGTCGTCTTCTTCTACCTGCTGTTGTTCTTGCACTTGCAG |
| | CTCGTGCACGACTTGGTGAACTACAAGATGCAGATGTAGTA (SEQ ID NO: 31) |
| 32 | TCTACAACTTGGAGATGCTGCTCCAGTAGAAGGTGCTGTTCTAGCAGAACATGGTCCAG |
| | AAGGACATCGTCTACTTGTAGTTGTTGTACATCGACTACTT (SEQ ID NO: 32) |
| 33 | AGATGTAGCACTACATGTTCAAGTTGTACCACTTGATGTTCTTCGTGTACGTGGTGTTG |
| | CTCCTGGAGGTGTTGATGTACAAGATGTACAACTTGGTCTT (SEQ ID NO: 33) |
| 34 | TCGAGATCTTCTACCACATGTAGTTGCTCCAGTTCGTGAAGATCCAGAACTTGTTGCAG |
| | GAGCTGCACTAGTACTACTTCAACTAGATCATGATGCAGGA (SEQ ID NO: 34) |
| 35 | TGTTCCAGTTCATCAAGGTCATCCTCCTCTAGTAGATGATGGAGTTGCTGATCCAGTAC |
| | CAGGTGTAGGAGTACGACTTGCTCGACCTGGTGAACGTCCT (SEQ ID NO: 35) |
| 36 | TGTAGGAGGTGTAGGTCCACAAGGTGTACAAGTACGTGGTCCACTTGTTGTACTAGATG |
| | GTGTACATCTTGTAGCTGATGGAGCACTACTTGCAGCAGAT (SEQ ID NO: 36) |
| 37 | TCGTGGTCCTGTTGTTCTACGAGCACTACGTGTAGGACATGGAGCTGAACTTGTTGGAG |
| | ATGGAGCACTACATGGTCCTCTTCATGGTGTACGTGCACAA (SEQ ID NO: 37) |
| 38 | TGTAGAAGGAGCTCATCTTGCTCAACCTGTTGTAGTAGCAGGACTTCTACGACATCGTC |
| | TACAACTTCATCCTGGTCATCTTCTAGCTGTTGCACTTCCA (SEQ ID NO: 38) |
| 39 | TCCAGTACCTGCTGTTGGACTACATGGACCTGTACGTGATCAAGTACATGCAGAAGGTC |
| | CTGTAGTACCTCTTGGTGCTGGTCCTCAAGATGATCGTGGA (SEQ ID NO: 39) |
| 40 | AGCTGTAGGTCGTCGTGCTGTACATGAAGCAGGTGGTGGTGTACGAGGTCGACTACCAG |
| | ATGGACGACGACAACCAGTTCGAGCTCGTGCTGCAGGTGTT (SEQ ID NO: 40) |
| 41 | TCATGTTCCTGGTGGTCATCTACATGATGTACTAGTTCAAGAAGTACGTCCTCTTCATC |
| | CACGAGAAGGTCGTCAACCACAACGTCTTGTAGCAGCTGAT (SEQ ID NO: 41) |
| 42 | AGTTGCTCAAGGACGAGCTCCACAAGAAGTTCGTGTTCAAGATGTAGTAGGAGATGATG |
| | CTGCTCCAGATGATCCAGTACAACCTGGACGTCGTGGACTT (SEQ ID NO: 42) |
| 43 | TCATCGACGAGGAGCAGATGATCAAGGTGTACAACGTCATCGTCTAGTACGAGAAGTTC |
| | TACGAGTAGATCATGGTCTTGTTCTTGAAGTTCAACATGTT (SEQ ID NO: 43) |
| 44 | TCAACCACGTCCACTACTTCTTCATGGTCTTGTAGTTGTACTTCTTGCTGCTCTTGCTC |
| | GTCAAGTTGGTGGTGTTCGTGTTCGAGCTGGAGCTCGAGAT (SEQ ID NO: 44) |
| 45 | ACAAGTAGTACAACTACTTCGTCGTGTTCTAGATCGACCACTACTTGTTGTTCTAGGTC |
| | GACCTCTACTACGACATCGTGCTGCACGACAAGGTCAAGTA (SEQ ID NO: 45) |
| 46 | TGATGTAGCTCATCATGATCTTCGTCAAGGTGAAGGTCATGTAGTTGTTGCACGACTTC |
| | GTGGAGCACTTGATGGACAACTTCGACGAGAACTTCTAGGA (SEQ ID NO: 46) |
| 47 | AGGTGTTGATCCTGCTCCACCTCATCGTGCTCCTGGTCTAGAAGTTGAACAAGAACATC |
| | AACAAGCTCTTCTTGAACAACGTCTAGTTGTTCTTGGTACA (SEQ ID NO: 47) |
| 48 | TCCACCTGAAGTTCGTCATCCACTACATCCACGTCTTGATGAACCTCCACAACGTCATG |
| | TTCGTGATGTTCCTGCAGATCCAGGTGGAGCTGTAGGTCGA (SEQ ID NO: 48) |
| 49 | ACTTCGAGTTCTTCCTCTTGGTCAAGAAGATCGTGTTCTTCTTGATGTAGAAGTTCCAC |
| | TACATCGTGGTGGAGTACGAGATCCTCCACCAGTTGAAGGA (SEQ ID NO: 49) |
| 50 | TCCTGCACTACCACCTCTTCGACTTCAACCTGGTCTTGCAGTAGTTCATGTTGGACGAC |
| | CTCTTCTTGGTCTACGAGTACGTCATGTTCTTCAAGTAGTA (SEQ ID NO: 50) |

### Chemical Agent

Examples of Projection-TAGs and associated agents and precursors thereof are described herein, and include pharmaceutically acceptable salts, and/or analogs thereof.

The formulas, analogs, and R groups can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -loweralkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms.

The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis &Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) P roc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original | Exemplary |
|---|---|
| Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of a target signal (or signals) by genome editing can result in protection from correlated diseases.

As an example, clustered regularly interspaced short palindromic repeats (CRISPR)/CRISP R-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for various diseases, disorders, or conditions to target cells by the removal or addition of signal activity (e.g., activation (e.g., CRISPRa), upregulation, overexpression, downregulation).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, E bola, lentivirus, or hybrids thereof.

### Gene Therapy Strategies.

| | Strategy |
|---|---|
| Viral Vectors | |
| Retroviruses | Retroviruses are RNA viruses transcribing |
| | their single-stranded |
| | genome into a double-stranded DNA copy, |
| | which can integrate into host chromosome |
| Adenoviruses (Ad) | Ad can transfect a variety of quiescent and |
| | proliferating |
| | cell types from various species and can |
| | mediate |
| | robust gene expression |
| Adeno-associated | Recombinant AAV vectors contain no viral |
| Viruses (AAV) | DNA and can carry ~4.7 kb of foreign |
| | transgenic material. They |
| | are replication defective and can replicate |
| | only while |
| | coinfecting with a helper virus |
| Non-viral vectors | |
| plasmid DNA | pDNA has many desired characteristics as a |
| (pDNA) | gene |
| | therapy vector; there are no limits on the size |
| | or genetic |
| | constitution of DNA, it is relatively |
| | inexpensive to supply, |
| | and unlike viruses, antibodies are not |
| | generated |
| | against DNA in normal individuals |
| RNAi | RNAi is a powerful tool for gene specific |
| | silencing that |
| | could be useful as an enzyme reduction |
| | therapy or |
| | means to promote read-through of a |
| | premature stop |
| | codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60 QC, for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of mapping, tracing, and/or profiling projection neurons in a subject in need thereof via administration of at least one Projection-TAG as disclosed herein.

Methods described herein are generally performed on a subject in need thereof. A subject in need of the therapeutic methods described herein can be a subject identified for and/or requiring projection neuron tracing in the brain. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of Projection-TAGs is, for example, an amount that would cause the desired therapeutic effect in a subject while minimizing undesired side effects. In various embodiments, an effective amount of Projection-TAGs described herein can substantially trace, map, and profile neuronal projections in the brain of the subject.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of Projection-TAGs can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to gain insight into the spatial location, gene expression, and chromatin accessibility profiles of diverse projection neurons.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the target region where the compound is administered; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4, ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, itis well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of one or more Projection-TAGs can occur as a single event or over a time course of treatment. For example, Projection-TAG scan be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for various diseases or conditions based on treatment need.

A Projection-TAG can be administered simultaneously or sequentially with another agent, such as an antibiotic, an anti-inflammatory, or another agent. For example, a Projection-TAG can be administered simultaneously with another agent, such as an antibiotic or an anti-inflammatory. Simultaneous administration can occur through administration of separate compositions, each containing one or more of a Projection-TAG, an antibiotic, an anti-inflammatory, or another agent. Simultaneous administration can occur through administration of one composition containing two or more of a Projection-TAG, an antibiotic, an anti-inflammatory, or another agent. A Projection-TAG can be administered sequentially with an antibiotic, an anti-inflammatory, or another agent. For example, a Projection-TAG can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies fora variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the one or more Projection-TAGs may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a Projection-TAG such as a compound may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In another non-limiting example, the compound may be administered at a concentration of 1.1-3.3×10¹² viral genomes/ml. The concentration of the compound may be diluted, the dilution comprising a 1×, 2×, 3×, or 4× dilution. An effective dose of the compound may depend on the region and route of administration. For example, a volume of 250 μl may be injected into the SC_{L}; 150 nl may be injected into the SC_{S}; and 500 nl may be injected into the MOp, SSp, VP, PAG, and MY.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Krrs

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to Projection-TAG components and/or precursors as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Projection-TAGs Enable Multiplex Projection Tracing and Multi-Modal Profiling of Projection Neurons

This example demonstrates the use of Projection-TAGs, a retrograde AAV platform, that allows multiplex tagging of projection neurons using RNA barcodes. Projection-TAGs can be leveraged to obtain a snapshot of activity-dependent recruitment of distinct projection neurons and their molecular features in the context of a specific stimulus.

Projection-TAGs have applications in building comprehensive multi-modal maps of brain neuronal cell types and their projections and may be used to inform therapeutic treatments.

### Introduction

Understanding brain function requires the elucidation of the complex wiring diagram and constituent cell types across brain regions. Revolutionary work from Golgi and Cajal laid the foundation for understanding the diversity of neurons and their anatomical connections based on morphological features. Over the past few decades, advancements have led to the further classification of neuronal cell types incorporating additional modalities, including molecular, electrophysiological, morphological, and anatomical features. Recent breakthroughs in high-throughput single-cell techniques and spatially-resolved molecular assays have sparked immense interest in developing a comprehensive multi-modal map of diverse neuronal cell types and their brain-wide projections. Despite the rapid integration of multiomic technologies in studying brain-wide connectivity, the investigation of spatially mingled neuronal projections continues to be hampered by the lack of broadly available tools to simultaneously trace multiple neuronal projections and/or profile projection neurons for multi-modal investigations.

Traditional neuroanatomical tracing methods, performed often with tracers or with viral vectors use fluorescence as the projection identifier, have been invaluable in mapping distinct neuronal projections. However, these methods are limited by the spectral properties of fluorophores that can be detected in a single experiment, thus limiting the number of neuronal projections that can be examined simultaneously. While the recent advance in fluorescence microscopy enables simultaneous imaging of up to 10 fluorophores, common microscopy equipment in neuroscience labs can reliably detect only 3-4 fluorophores, limiting the number of projections to be examined simultaneously. Furthermore, such approaches are not directly suited for high-throughput sequencing-based assays such as single-cell RNA sequencing (scRNA-seq) and single-cell assay for transposase-accessible chromatin with sequencing (scATAC-seq), as the detection of exogenous transcripts is usually low by short-read sequencing. Alternatively, one can still build a multi-modal atlas for projection neurons by tracing one or two projections at a time using fluorophores and incorporating this tracing paradigm with fluorescently-activated cell sorting (FACS) for single-cell profiling. Methods, such as retro-seq and epi-retro-seq, characterized the molecular features of projection neurons, but they are often inefficient, costly, and not suitable for studying the complex wiring diagrams of projections to multiple targets.

RNA barcode-based tracing tools provide a promising avenue for high-throughput neuroanatomical studies, as diverse RNA barcodes can be parallelly detected via sequencing or imaging, thus offering a very powerful and scalable strategy to trace neuronal projections. Employing an anterograde tracing scheme, MAPSeq and BARseq utilize a sindbis viral library to encode a diverse collection of short random RNA barcodes, which are strategically designed to label individual cells and engineered to be anterogradely transported to axonal terminals for projection tracing. Despite the ability to quantitatively measure the projection strength to any target regions of investigation, the requirement of highly customized instruments and pipelines by those methods has restricted their adoption beyond several expert labs. The cellular toxicity associated with the sindbis virus also poses a challenge to integrating their use with chronic experimental paradigms. More recently, employing a retrograde tracing scheme using adeno-associated viruses (AAVs), both Projection-seq and MERGE-seq used RNA barcodes as the projection identifier for multiplex projection tracing. The low cellular toxicity of AAVs allowed them to correlate gene expression to projections using scRNA-seq, but their compatibility for additional modalities, such as epigenetic profiling, spatial analysis, and activity-dependent circuit mapping is lacking or not sufficiently demonstrated.

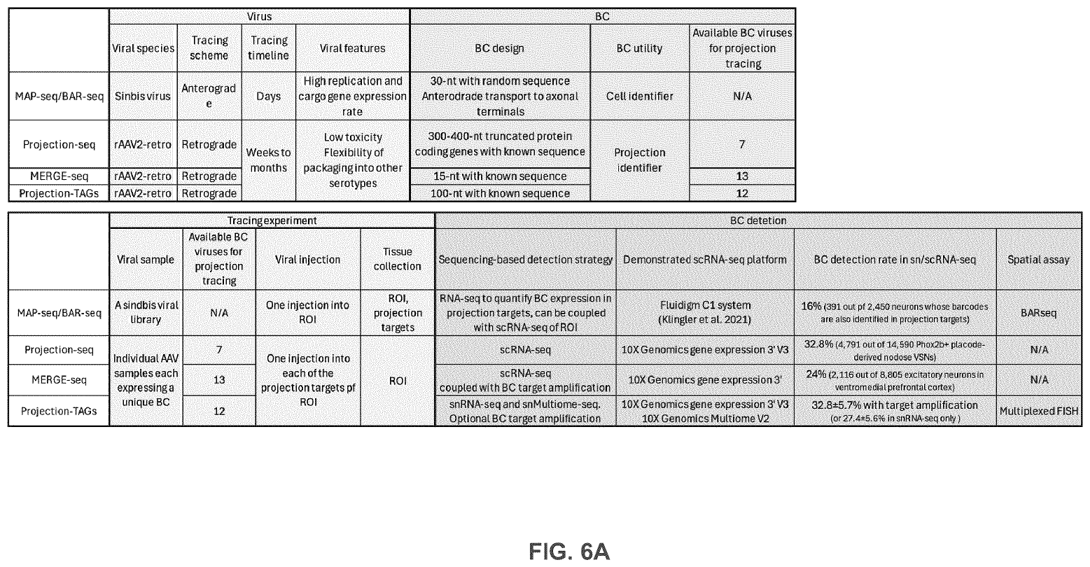

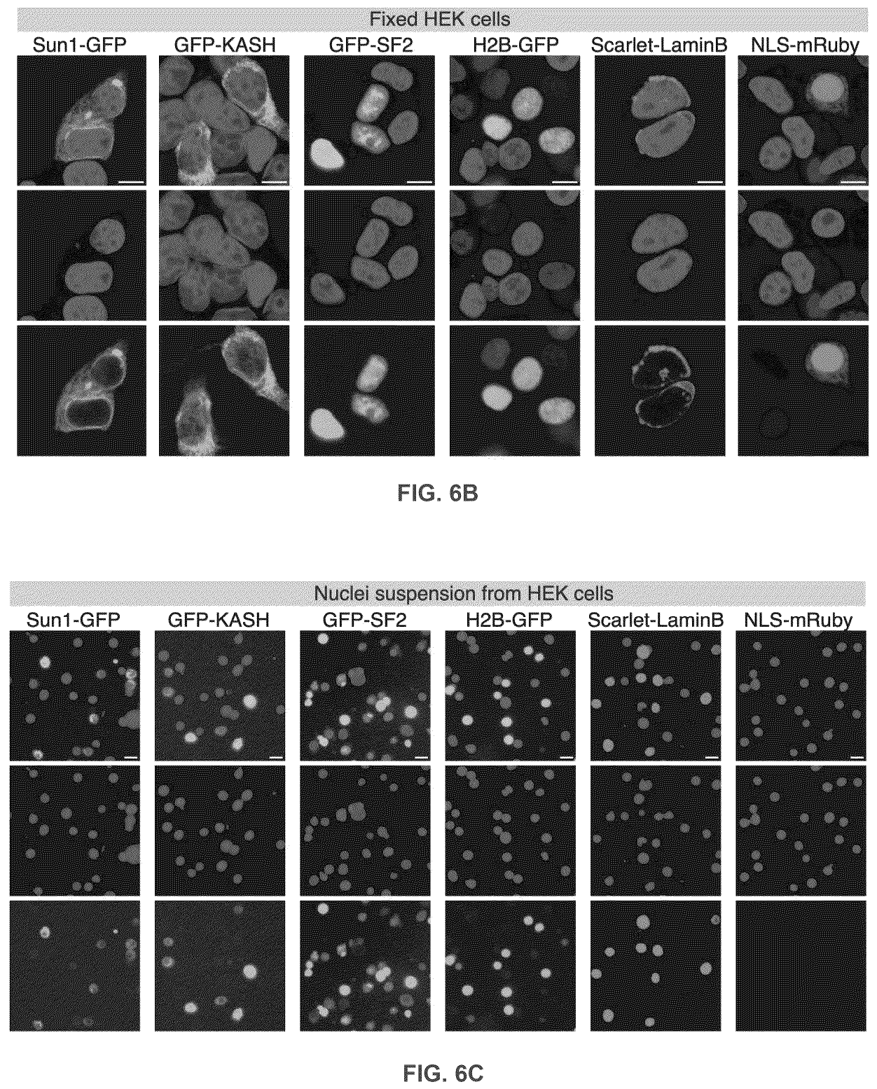

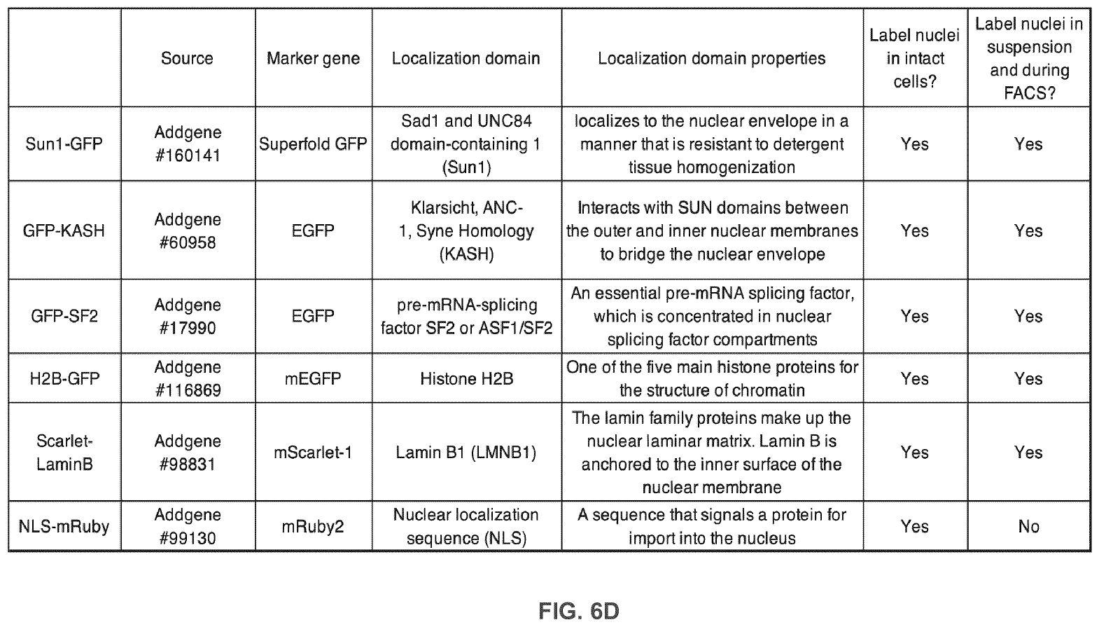

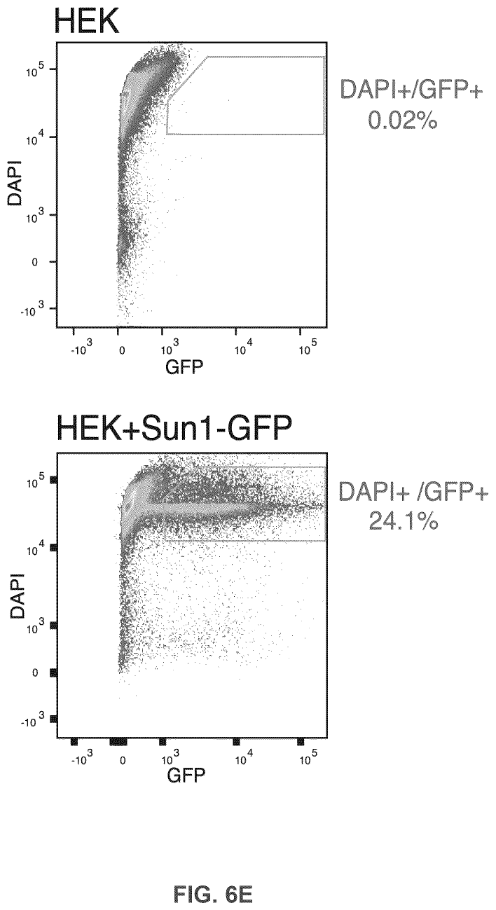

Herein Projection-TAGs are introduced: a retrograde AAV platform that allows multiplex tracing of projection neurons using RNA barcodes, herein designated as Projection-TAGs. Similar to Projection-seq and MERGE-seq, the key component of Projection-TAGs is a set of engineered retrograde AAVs each expressing a unique RNA barcode TAG, which acts as the projection identifier (FIG. 6A). With this scheme, neurons projecting to a target region are uniquely labeled by a retrograde AAV-mediated TAG and multiplex projection tracing can be achieved by injecting a unique Projection-TAG AAV into each of the target regions predefined for investigation. A toolbox was developed for Projection-TAG detection using various commercial assays, enabling multiplex spatial neuroanatomical studies, high-throughput multi-modal profiling of projection neurons, and investigation of activity-dependent populations in response to a stimulus of interest. Herein, Projection-TAGs were utilized to study the complex wiring diagram, spatial organization, transcriptional, and epigenetic landscapes of intracortical-, subcortical-, and corticospinal-projecting neurons in the cortex and identify activity-dependent recruitment of distinct cortical projections in a mouse model of visceral pain. Projection-TAGs are designed for incorporation into existing experimental pipelines with minimal effort and easy application in studying the nervous system.

### Results

### Design of Projection-TAGs

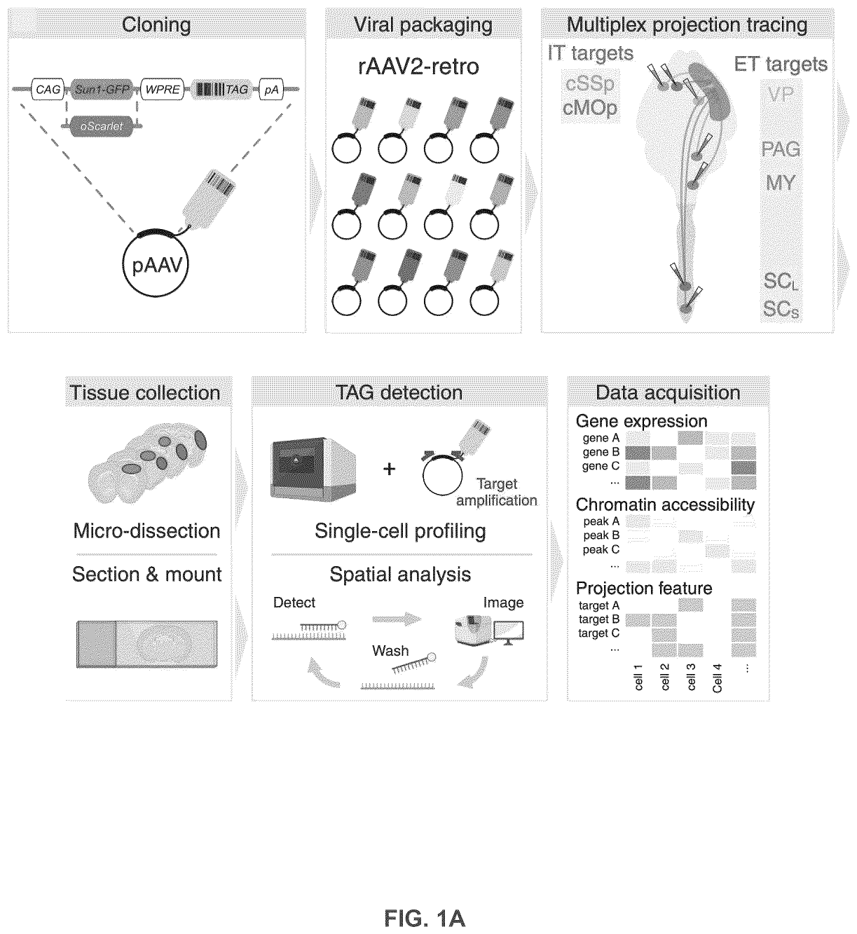

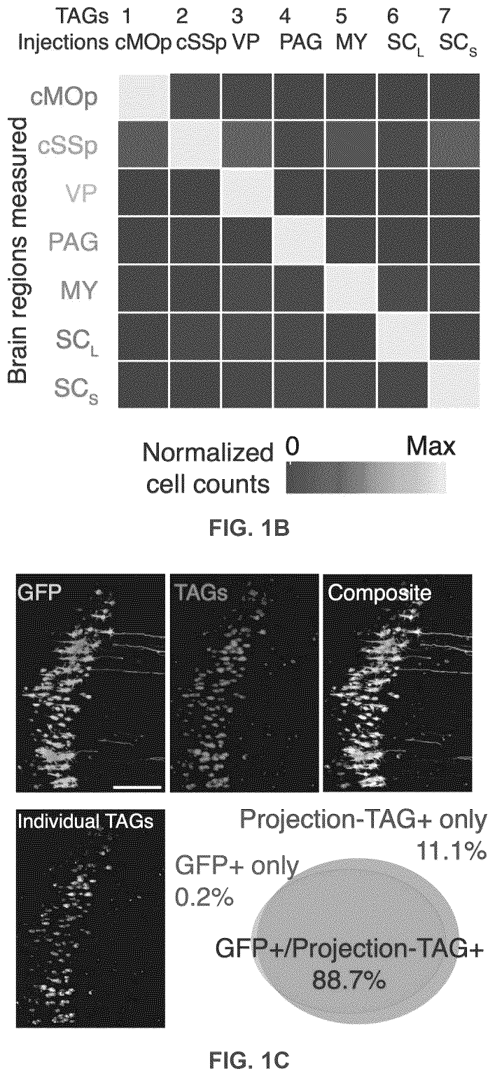

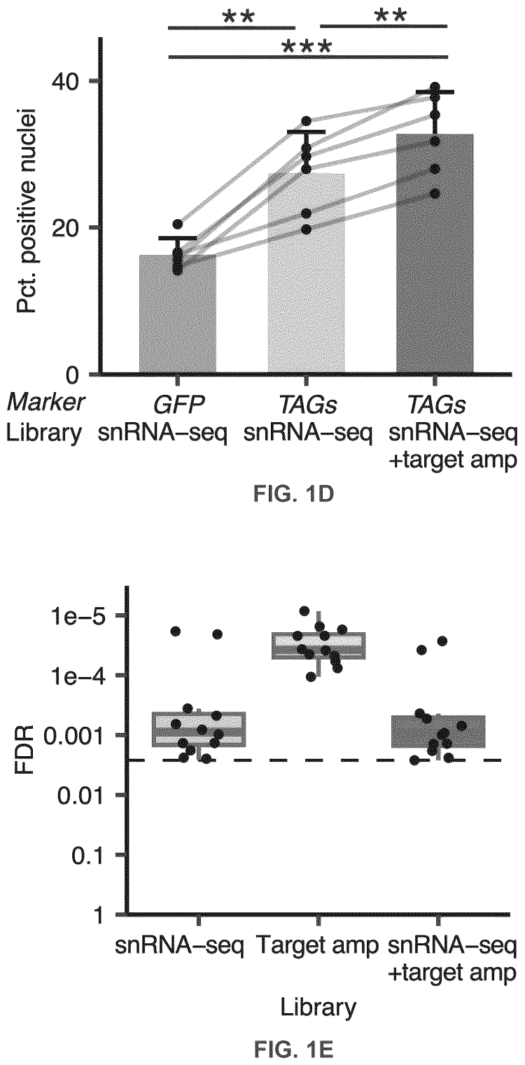

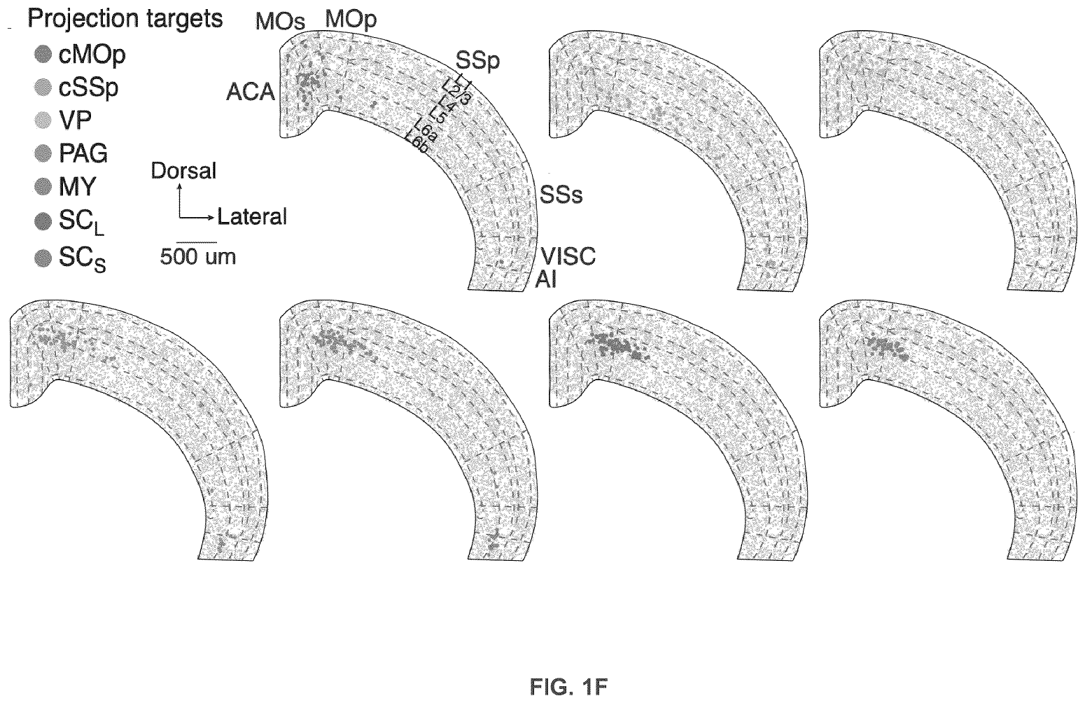

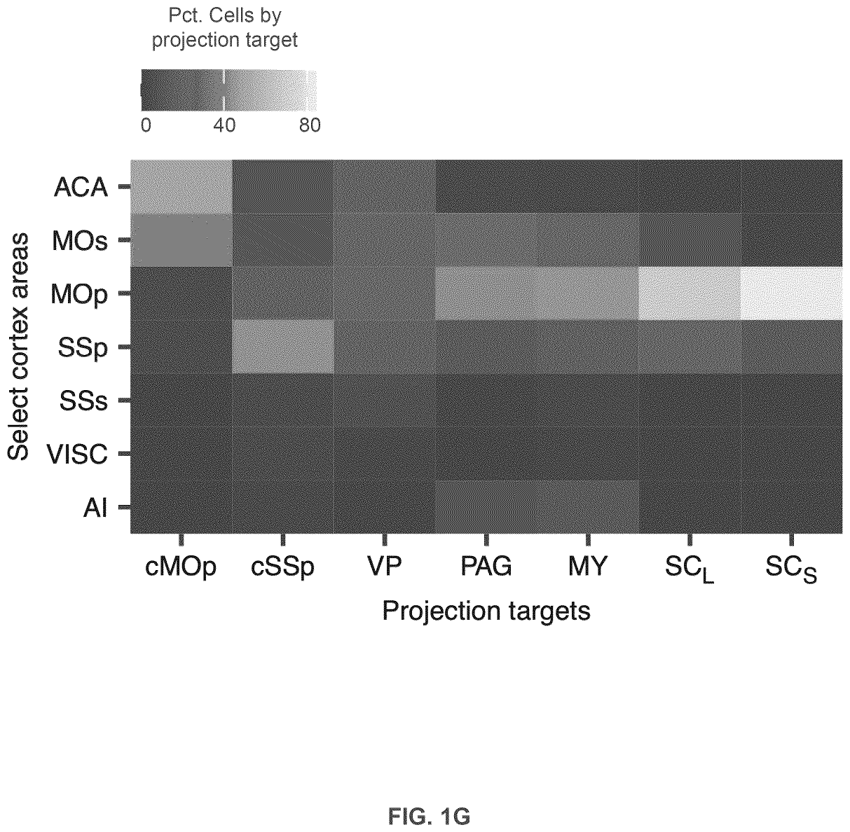

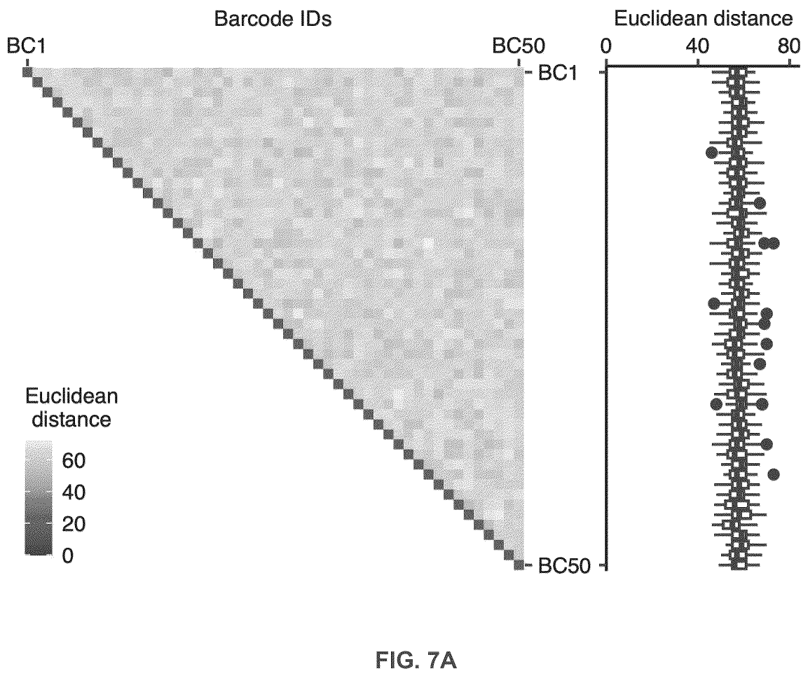

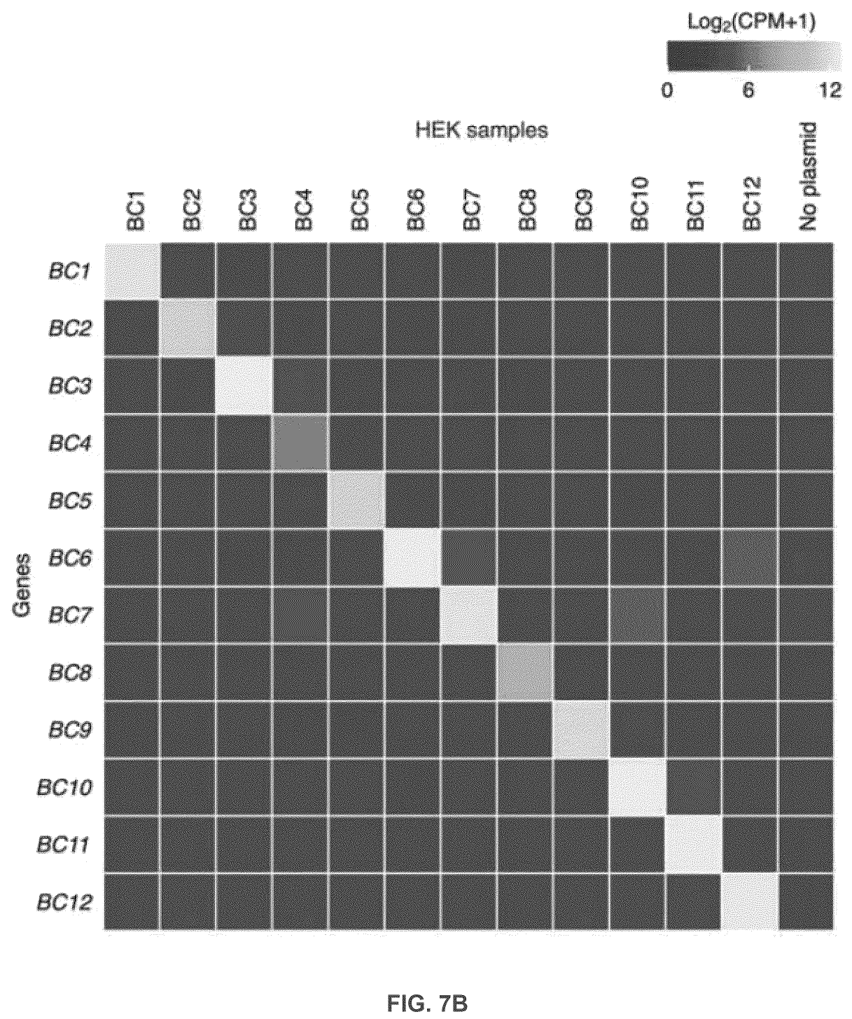

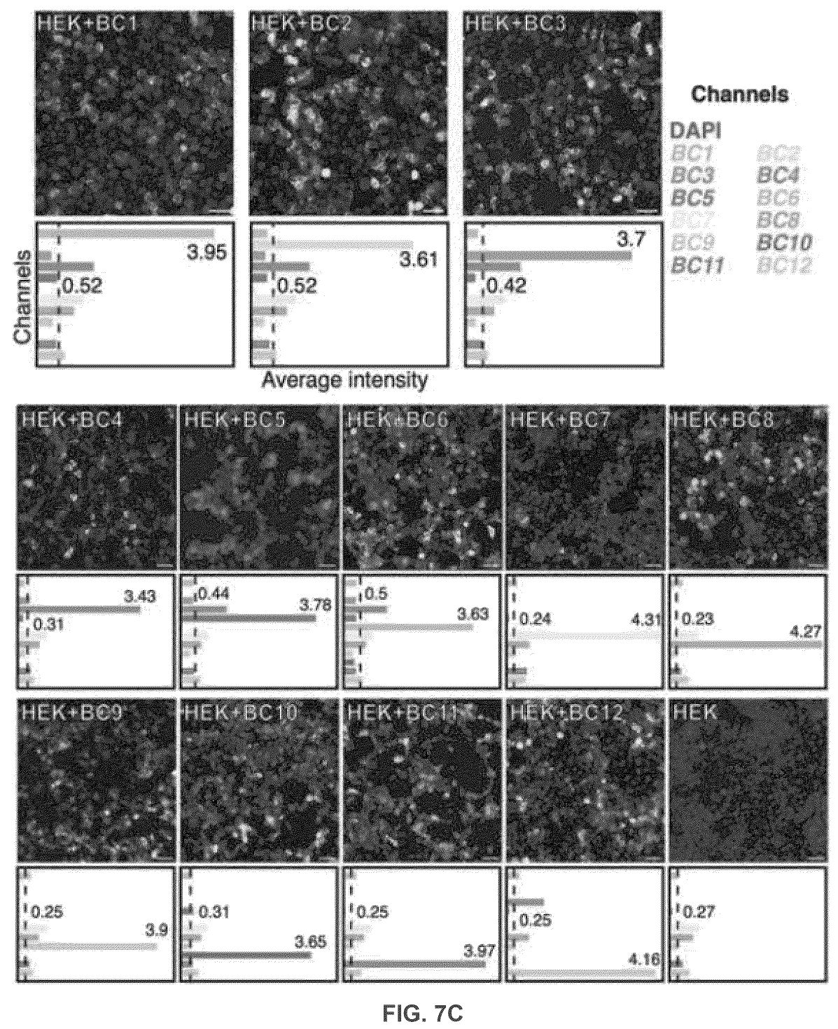

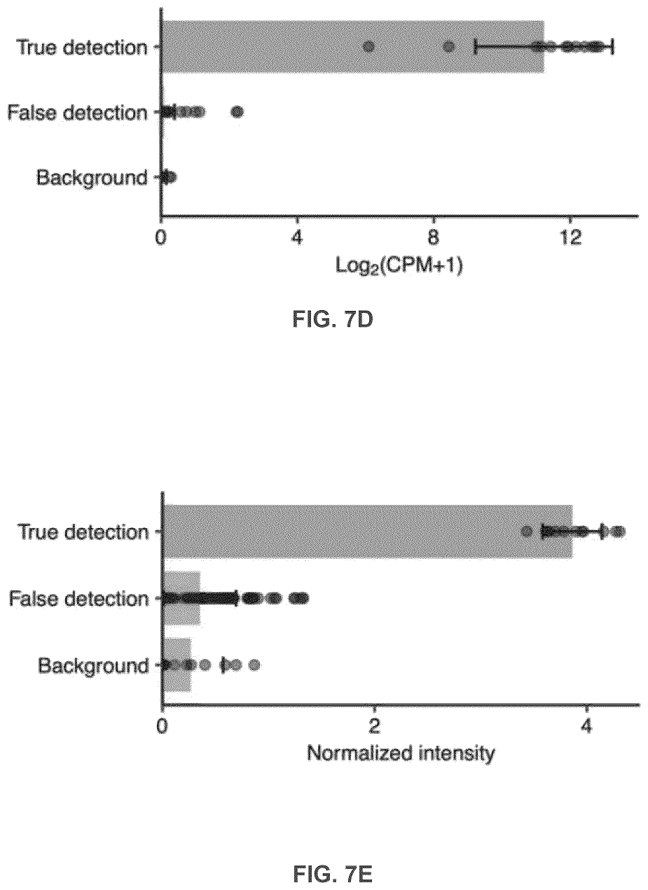

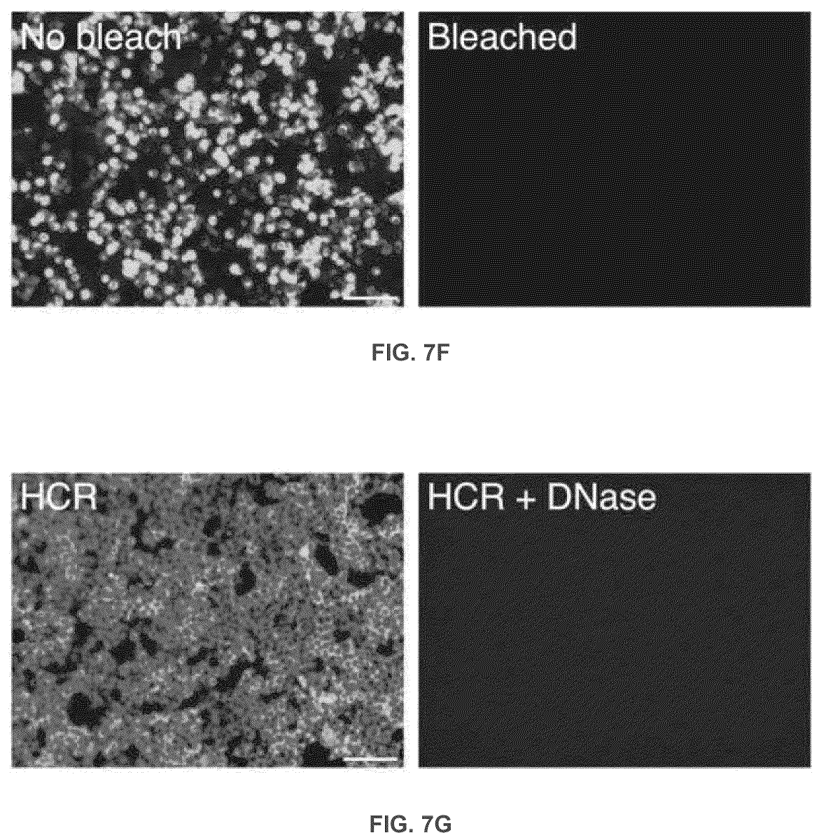

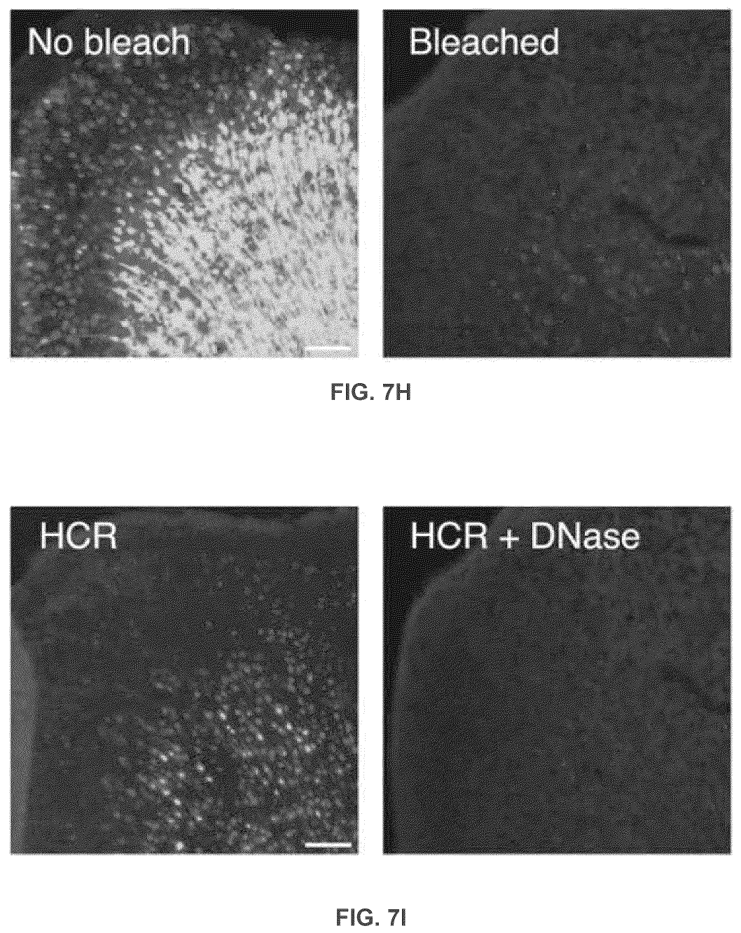

To democratize and facilitate the use of Projection-TAGs in neuroscience labs without any specialized equipment, the following features were incorporated into the Projection-TAG plasmid design: First, a chicken beta-actin (CAG) promoter was employed to enable ubiquitous Projection-TAG expression in various tissue and cell types (FIG. 1A). Second, a fluorescent marker was included to enable flexibility in downstream processes and confirming viral expression. Several fusion fluorescent proteins were screened for the ability to label both intact cells and nuclei in suspension (FIG. 6B, FIG. 6C), and included in AAV plasmids a fusion of Sad1 And UNC84 Domain Containing 1 (Sun1) to Green Fluorescent Protein (GFP), allowing enrichment of target cell/nucleus populations using FACS (FIG. 6E). Sun1-GFP labels the nuclear membrane without interfering with the chromosome structure and has been employed in numerous epigenetic studies (FIG. 6D). To further enhance Projection-TAG functionality, a protocol was developed for photobleaching Sun1-GFP fluorescence (FIG. 7F, FIG. 7H) and created a version of Projection-TAGs expressing a red fluorophore oScarlet (FIG. 1A). Third, to enable projection tracing using various commercial assays, 50 unique 100-nt RNA barcodes were cataloged (FIG. 7A, see also SEQ ID NO: 1-50). By insertion into the 3′ untranslated region (UTR) of the Sun1-GFP transcript, 12 plasmids were generated, each expressing a unique Projection-TAG (TAGs 1-12, FIG. 1A). This design aimed to optimize its detection by 3′ end RNA-seq assays and enable the development of fluorescent in situ hybridization (FISH) probes for spatial analysis.

According to embodiments described herein, a promoter can be selected from CAG, Ef1a, Syn, and promoters known to those of ordinary skill at the time of filing. According to embodiments described herein, a fluorescent marker/reporter/protein can be selected from GFP, oScarlet, YFP, RFP, and fluorescent markers/reporters/proteins known to those of ordinary skill at the time of filing.

As a proof of principle to validate Projection-TAG expression and detection, RNA-seq was first performed and multiplexed FISH on human embryonic kidney (HEK) cells transfected with individual Projection-TAG plasmids (FIG. 7B, FIG. 7C). Projection-TAGs were detected with high specificity, as the detection of Projection-TAGs expressed in each HEK sample is at least 422.4 times (RNA-seq) and 6.9 times (multiplexed FISH) greater than that of Projection-TAGs not expressed or the background (FIG. 7D, FIG. 7E). To use Projection-TAGs for multiplex tracing of neuronal projections in vivo, validated plasmids were packaged into recombinant AAV serotype 2 retro (rAAV2-retro) and generated a set of 12 Projection-TAG AAVs each expressing a unique Projection-TAG (FIG. 1A).

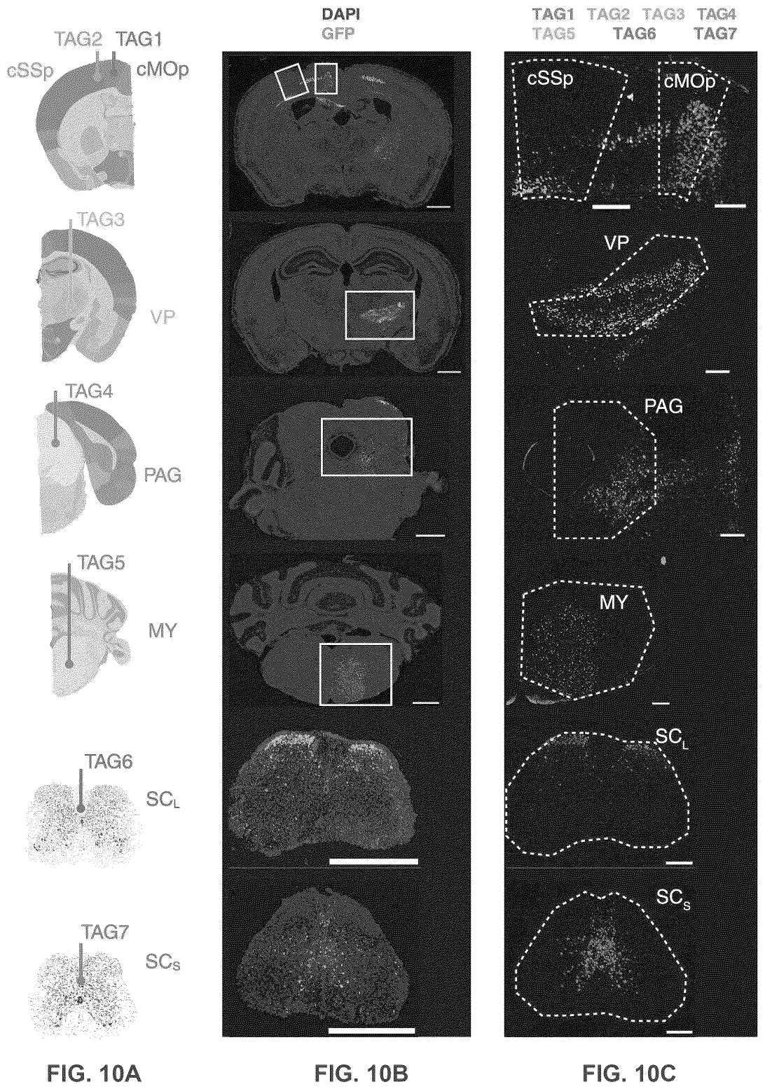

To evaluate the ability to use Projection-TAGs in high-throughput multi-modal profiling of projection neurons, multiplex projection tracing was applied in the adult mouse primary motor (MOp) and primary somatosensory cortex (SSp). Neurons in the MOp and SSp are anatomically organized by cortical layers and exhibit distinct layer-specific connectivity with other brain regions and have been extensively known to coordinate a wide range of innate and learned behaviors. Tremendous progress has been made to characterize the diverse MOp and SSp cell types using single-cell multiomic approaches and create a comprehensive molecular taxonomy of projection neurons and their brain-wide projections, allowing validation of the efficacy of Projection-TAGs by comparing the data generated herein with the ground truth data. To test Projection-TAGs in vivo, seven downstream projection targets of MOp and SSp were identified, including two intratelencephalic (IT) targets (contralateral MOp [cMOp] and contralateral SSp [cSSp]) and five extratelencephalic (ET) targets, which can be further classified into three subcortical targets (ipsilateral ventral posterior nucleus of the thalamus [VP], ipsilateral periaqueductal grey [PAG], ipsilateral medulla [MY]) and two corticospinal targets (lumbar spinal cord [SC_{L}], and sacral spinal cord [SC_{S}], FIG. 1A, FIG. 10A).

Multiplex Projection Tracing Using Projection-TAG rAAV2-Retro

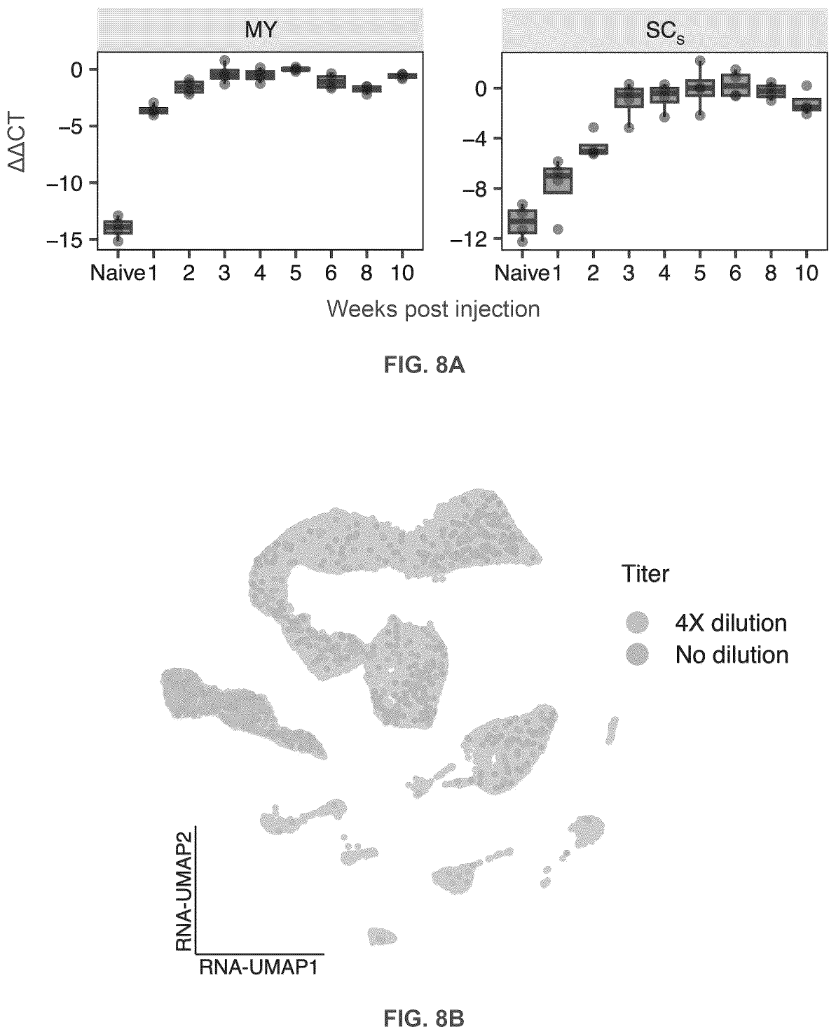

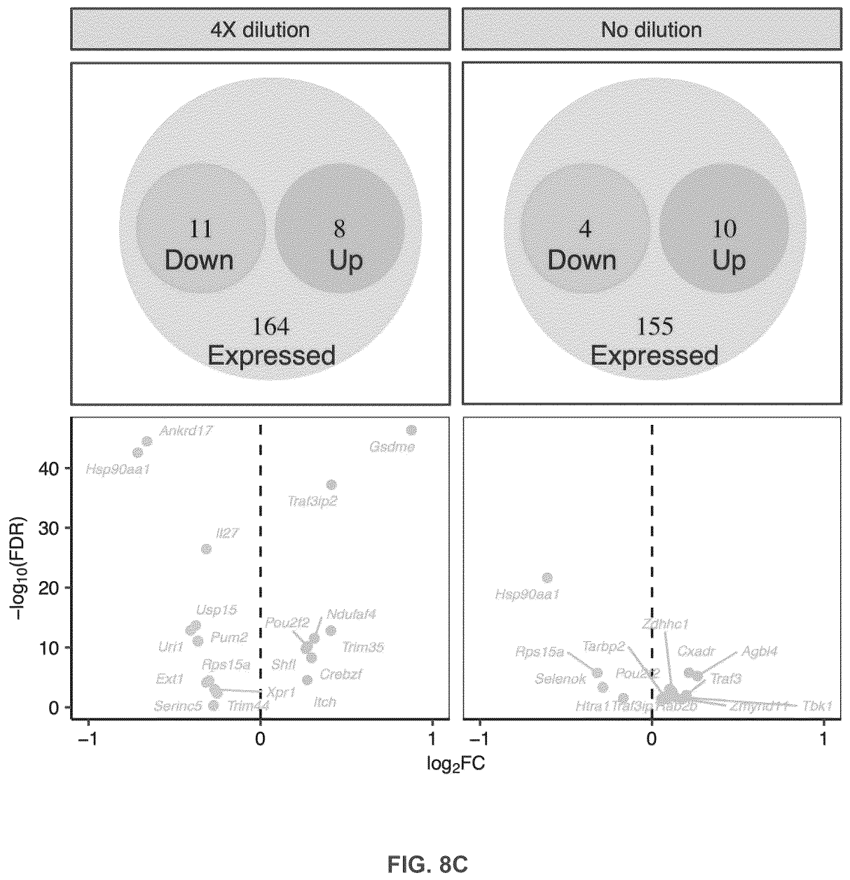

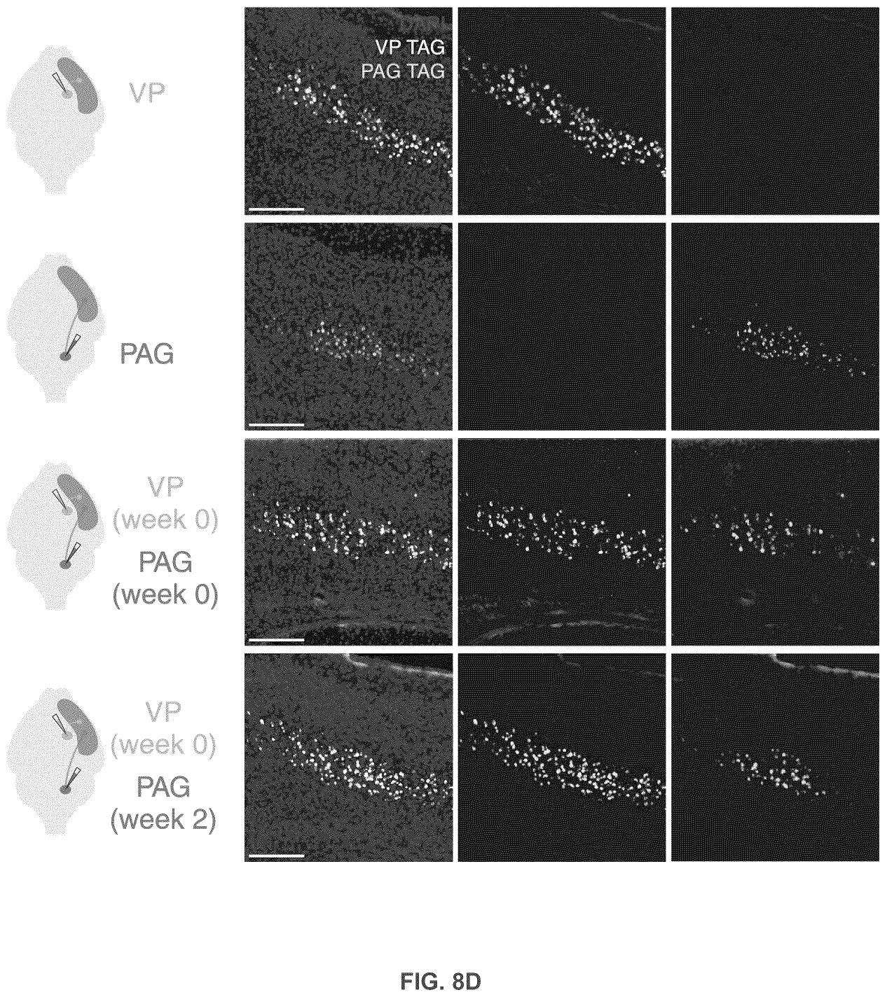

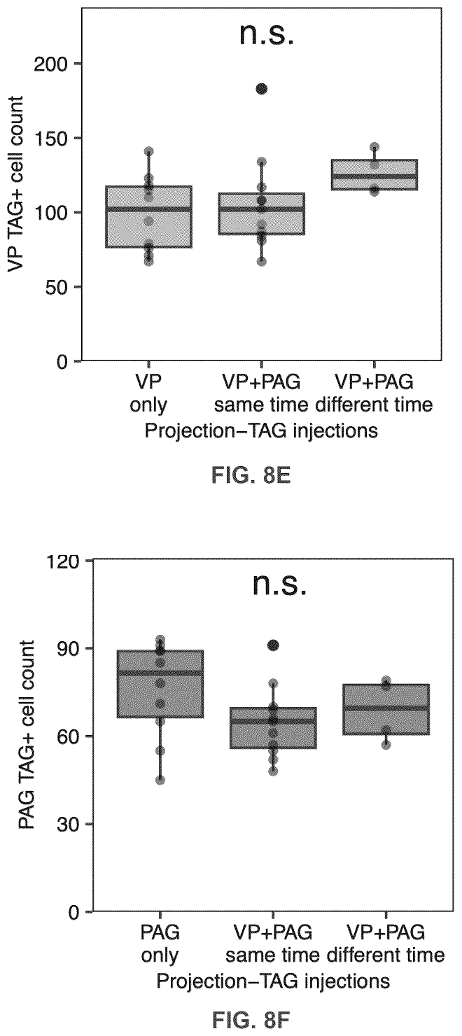

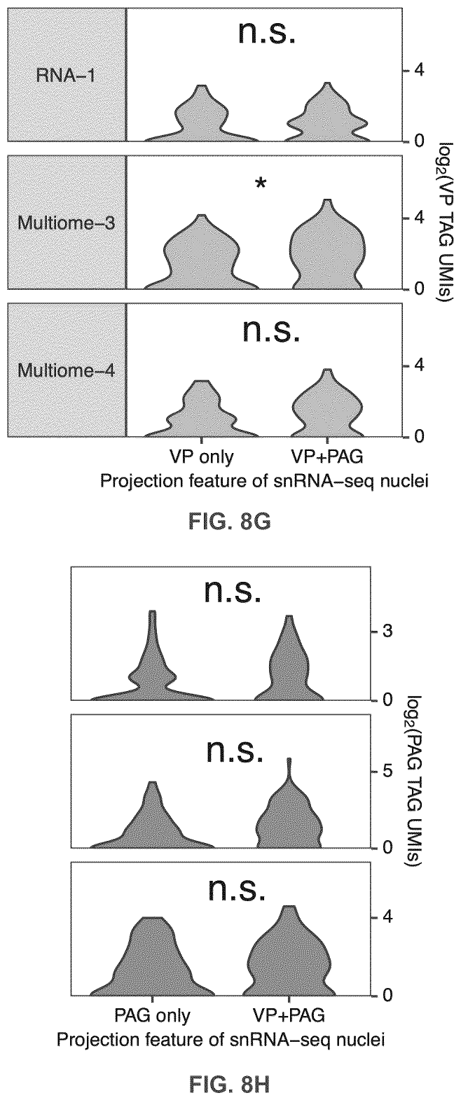

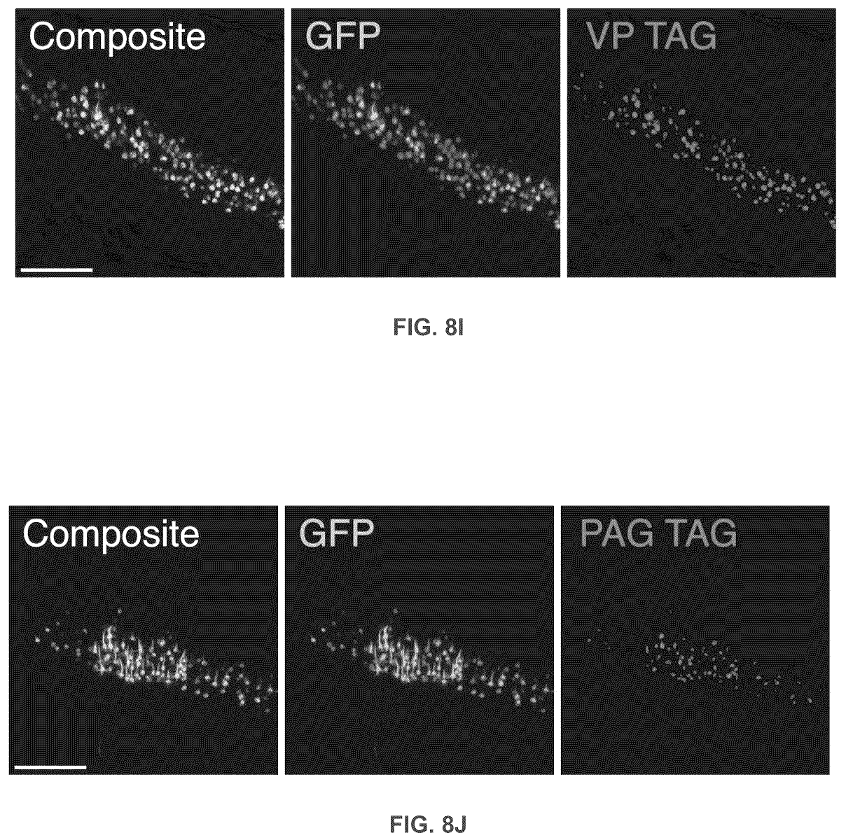

While previous studies using rAAV2-retro have shown that two weeks are typically sufficient for fluorescently labeling cortical projections, a multiplex tracing experiment would benefit from understanding the temporal kinetics and stability of cargo gene expression across different cortical projections, which remains largely elusive. Thus, Projection-TAG expression was investigated in two cortical projections with notably long axons that may represent the upper limit of waiting time: MY, one of the longest cortical projections in the brain, and SC_{S}, one of the longest cortical projections in the central nervous system. A Projection-TAG AAV was injected into either MY or SC_{S} and quantified the Projection-TAG expression overtime in the cortex using qPCR (FIG. 8A). Projection-TAG expression is detectable as soon as one week after injection and increased at week two, consistent with previous reports. However, the expression in mice receiving MY injection increased rapidly and reached its peak at week three, whereas the expression in mice receiving SC_{S} injection increased more steadily and peaked at around week five. In both cases, expression plateaued until week ten, the endpoint of the study. While the initial peak timing may vary across projections, results suggest that the stable Projection-TAG expression after peaking provides a flexible time window for synchronizing peak expression in different projections and coupling Projection-TAGs with other experimental paradigms.

To make sure Projection-TAGs can be unbiasedly used for multiplex projection tracing, it was next investigated if significant viral competition exists among Projection-TAG AAVs that may reduce the tracing efficiency. Prior experiments showed that, in the medial part of the posterior parietal association area of the cortex (PTLp), more than 50% of neurons projecting to PAG also project to VP. Competition between Projection-TAG AAVs should reduce tracing efficiency in mice receiving both VP and PAG injections compared to mice receiving only one of the injections. However, neither VP- nor PAG-TAG+ cell counts are significantly different from mice receiving either injection than mice receiving both injections, regardless of whether the injections were made simultaneously or separately (FIG. 8D, FIG. 8E, FIG. 8F). Additionally, VP- and PAG-TAG UMI counts are not significantly different in most snRNA-seq libraries from nuclei expressing one of the TAGs than nuclei co-expressing both TAGs (FIG. 8G, FIG. 8H). These results suggest that the Projection-TAG expression is largely unaffected by viral competition and thus can be safely used for multiplex tracing.

Moreover, Projection-TAG AAVs were tested for altered gene expression and induced immune response in infected cells. In snRNA-seq, Projection-TAG+ nuclei (nuclei with >0 UMI for any Projection-TAGs) co-cluster well with Projection-TAG− nuclei on the UMAP, indicating little gene expression alteration due to AAV infection (FIG. 8B). Gene ontology analysis revealed no significant immune response to viral infection in Projection-TAG+ nuclei compared to Projection-TAG− nuclei (FIG. 8D). Therefore, rAAV2-retro stably express Projection-TAGs with minimal immune response.

Detection of Projection-TAGs with High Specificity and Efficiency

Multiplex projection tracing relies on the demultiplexing of individual Projection-TAGs with various detection assays. Herein, the specificity and efficiency of Projection-TAG detection in multiplexed FISH and snRNA-seq was examined and considerations were highlighted as potentially affecting the interpretation of Projection-TAG experimental results.

To examine the specificity of Projection-TAG detection in vivo, multiplexed FISH was performed on brain sections from mice receiving Projection-TAG AAV injections into the projection targets of the cortex (FIG. 1A, FIG. 10A). A high correspondence was observed in Projection-TAG detection at the injection sites with 19.5-393 times greater cells labeled with Projection-TAGs injected into a given region than those injected into other regions (FIG. 1B, FIG. 10B, FIG. 10C). The efficiency was next compared of Projection-TAG labeling to fluorescent labeling by examining MOp cells retrogradely labeled with Projection-TAGs in multiplexed FISH and those labeled with the Sun1-GFP fluorescence, expressed by the same Projection-TAG plasmids. A high degree of overlap was observed between Projection-TAG+ cells and Sun1-GFP+ cells (FIG. 1C, FIG. 8I, FIG. 8J), despite Sun1-GFP and Projection-TAGs exhibiting distinct subcellular compartmentalization (FIG. 1C). Therefore, Projection-TAG labeling demonstrated high specificity and efficiency in multiplexed FISH, and that GFP fluorescence can serve as a surrogate to estimate Projection-TAG+ cells in vivo.

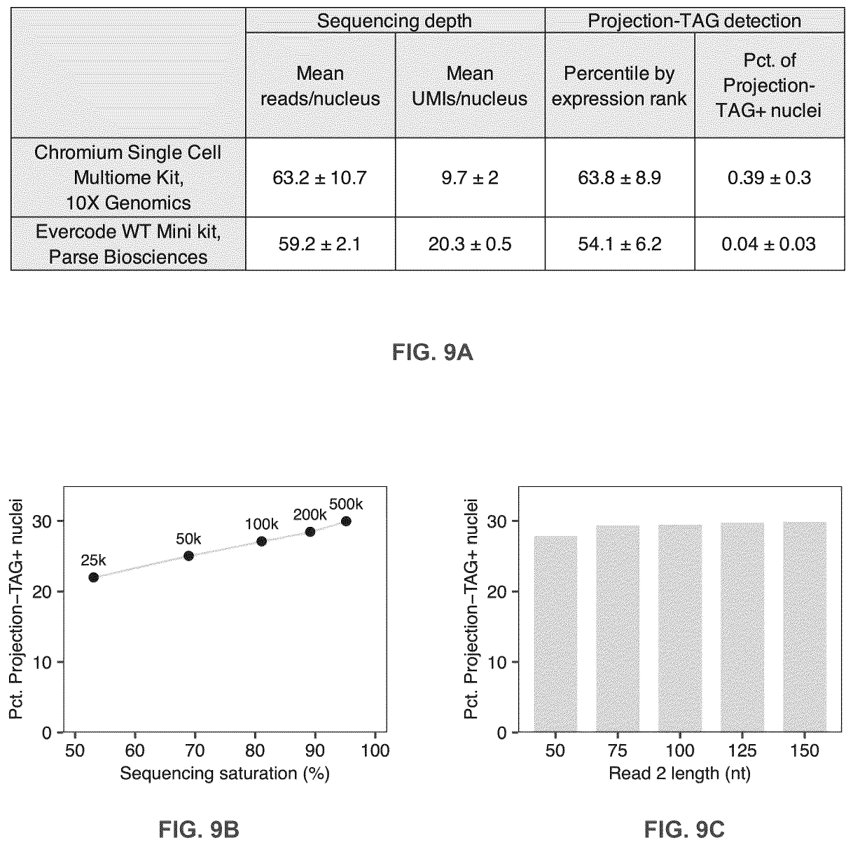

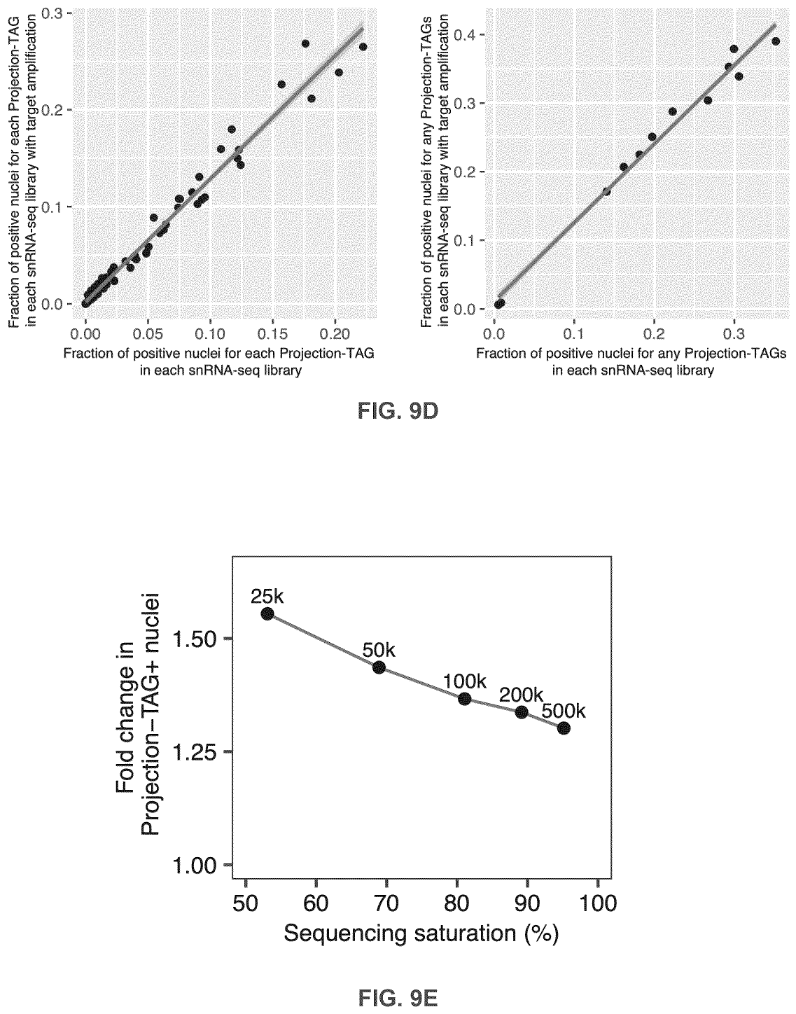

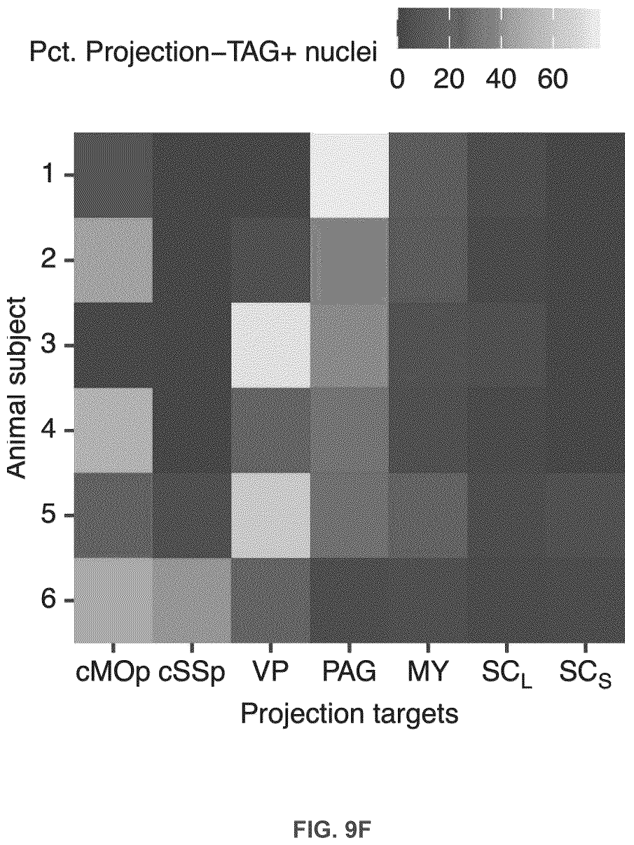

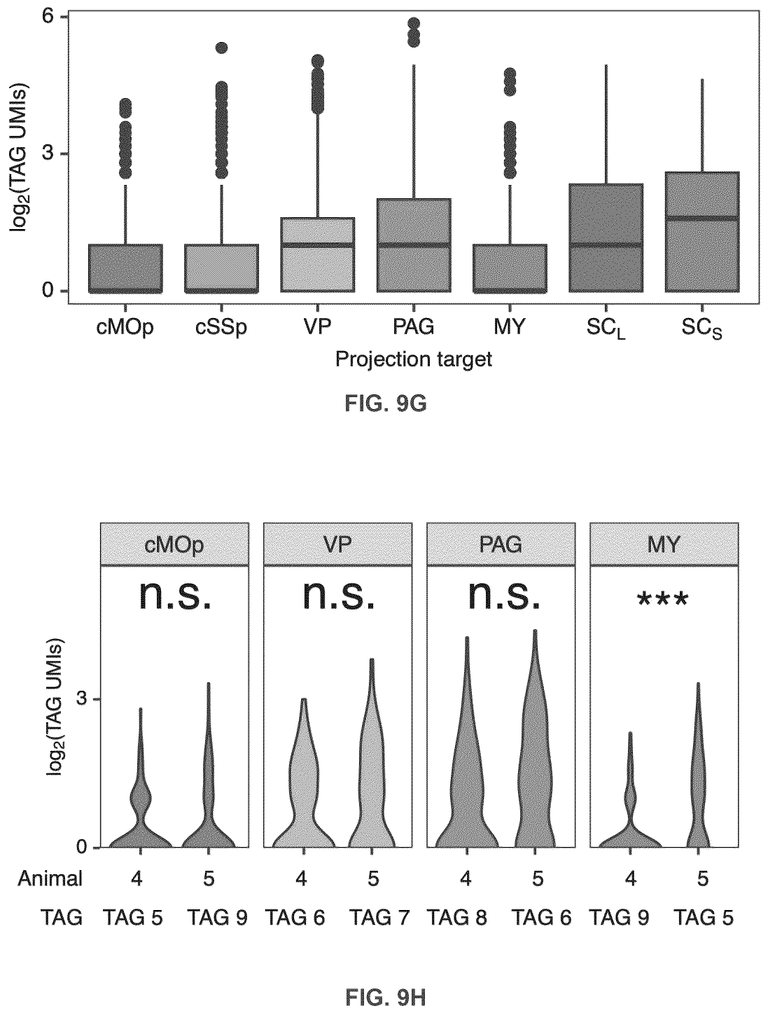

As different 3′ scRNA-seq technologies use varying strategies to capture RNA transcripts and construct sequencing libraries, which may affect the 3′ gene feature detection in sequencing, the Projection-TAG detection was next compared by splitting one nuclear resuspension sample into two scRNA-seq assays. The 10× Genomics assay yielded higher Projection-TAG detection and identified proportionally more Projection-TAG+ nuclei compared to that from Parse Biosciences, despite lower UMI recovery (FIG. 9A). Therefore, the 10× Genomics assay was used for the sequencing experiments described herein. Besides sequencing assays, sequencing setups also affect Projection-TAG detection. At least 100 k reads/nucleus were targeted or 80% saturation rate for library sequencing, which recovered 85.2% of the Projection-TAG UMIs and 90.5% of Projection-TAG+ nuclei compared to sequencing at 500 k reads/nucleus (FIG. 9B). Sequencing read 2 length exceeding 75-nt had little effect on Projection-TAG detection (FIG. 9C). To further improve detection and reduce overall sequencing cost, a PCR-based protocol was devised that target amplifies Projection-TAG UMIs from the cDNA library (Methods). Target amplification increased the discovery of Projection-TAG+ nuclei by 1.2±0.1 fold and detection of positive nuclei for individual Projection-TAGs by 1.4±0.6 fold per library (FIG. 9D). Notably, target amplification recovered proportionally more Projection-TAG+ nuclei when the library is shallowly sequenced (FIG. 9E), while maintaining high Projection-TAG detection.

To examine the efficiency of Projection-TAG detection, snRNA-seq was performed on FACS sorted nuclei, in which >95% of the nuclei are GFP+, from mice receiving Projection-TAG AAV injections into the projection targets of the cortex (FIG. 1A). Significantly more Projection-TAG+ nuclei were identified (32.8±5.% with target amplification, 27.4±5.6% in snRNA-seq library alone) than Sun1-GFP+ nuclei (FIG. 1D). To investigate the specificity of Projection-TAG detection, the Projection-TAG mismatching was assessed in snRNA-seq. Projection-TAGs used in experiments are highly expressed (75.9±6.6 percentile by expression ranks among all genes) while Projection-TAGs not used yielded zero counts in both snRNA-seq library and target amplification. The false positive rate of Projection-TAG detection was also assessed due to ambient RNA contamination and clustering and annotation errors. Projection-TAGs were detected in only 0.1±0.08% of the empty droplets per library (FIG. 1E) and 0.32% of the non-neuronal cells. Target amplification marginally increased the average Projection-TAG detection in empty droplets and non-neuronal cells to 0.104% and 0.34%, respectively. These data suggest that in snRNA-seq, Projection-TAG detection is highly specific and more efficient compared to the detection of GFP expressed by the same plasmid. To control for false discovery due to technical artifacts, the FDR was applied at 0.34% in the downstream Projection-TAG analyses.

However, one should be mindful of several limitations when interpreting the Projection-TAG results. First, while the Projection-TAG detection in multiplexed FISH assay is highly efficient (only 0.2% GFP+ cells do not express Projection-TAGs), the false negative rate of Projection-TAG detection is nontrivial in snRNA-seq (67.2%±5.7% nuclei from snRNA-seq libraries prepared by FACS do not express Projection-TAGs). Consequently, a lack of Projection-TAG expression in a snRNA-seq cell should not be simply interpreted as lack of projection. In addition, technical and biological variables may introduce bias in Projection-TAG tracing efficiency. For example, the distribution of cells labeled for each projection differs across animals (FIG. 9F), likely due to variations of stereotaxic injections, and the Projection-TAG expression is different across projection pathways (FIG. 9G). However, the exact TAGs used to trace each projection pathway did not significantly affect their expression in most cases (FIG. 9H), suggesting Projection-TAGs can be used interchangeably in tracing experiments.

### Spatial Organization of Projection Neurons Across the Cortex

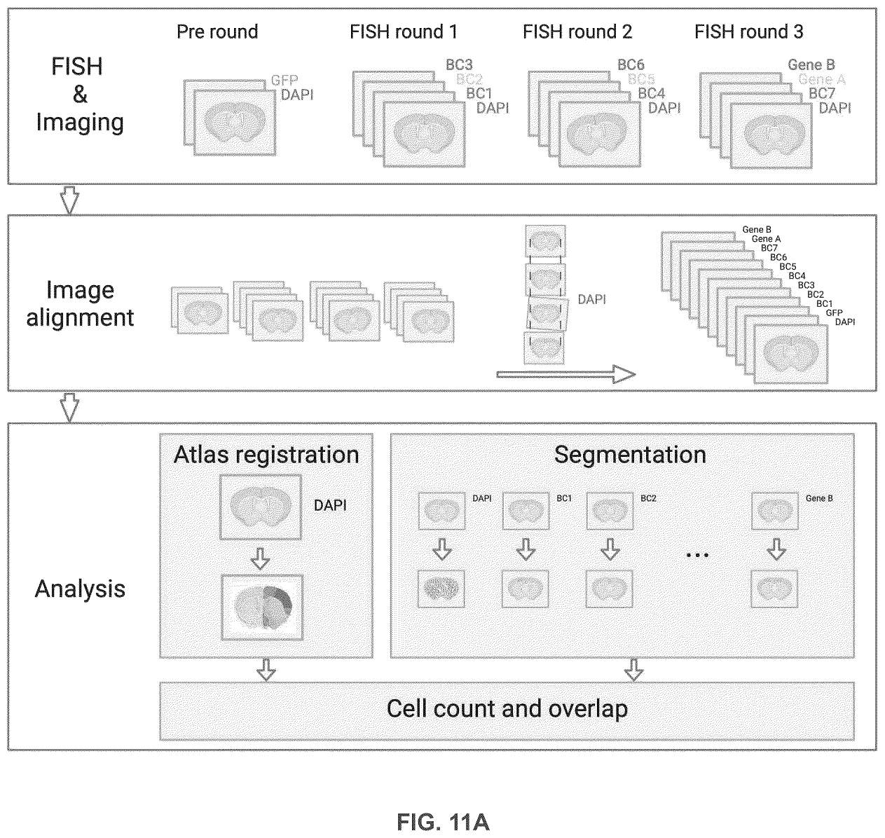

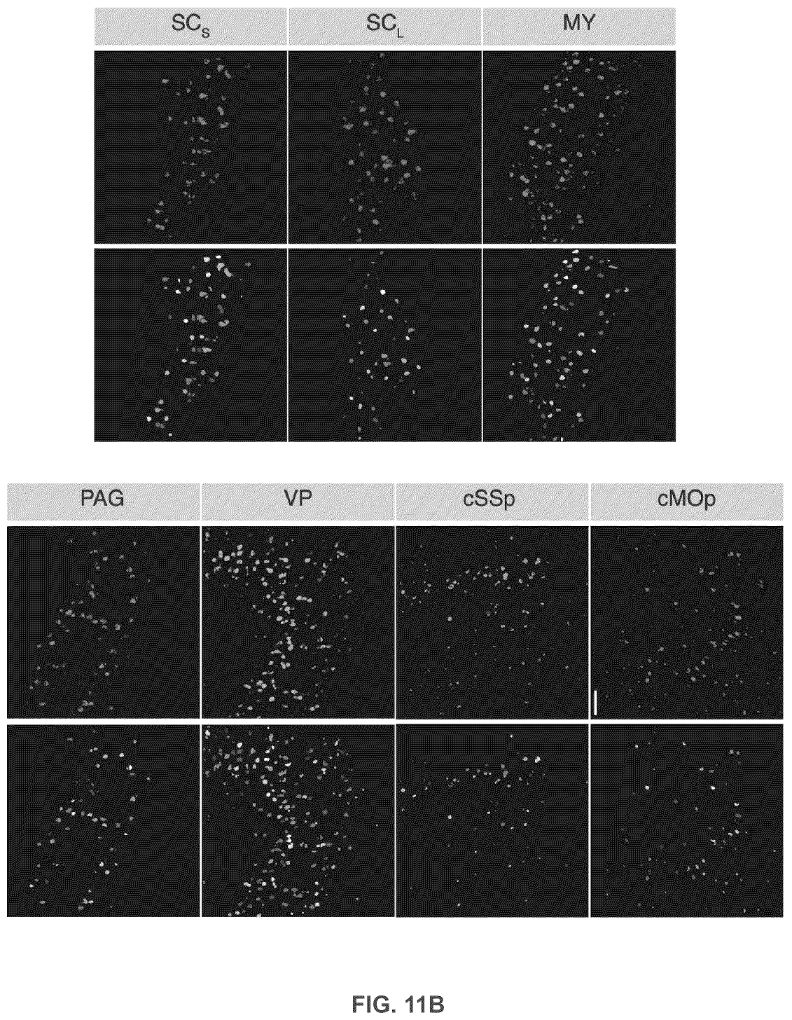

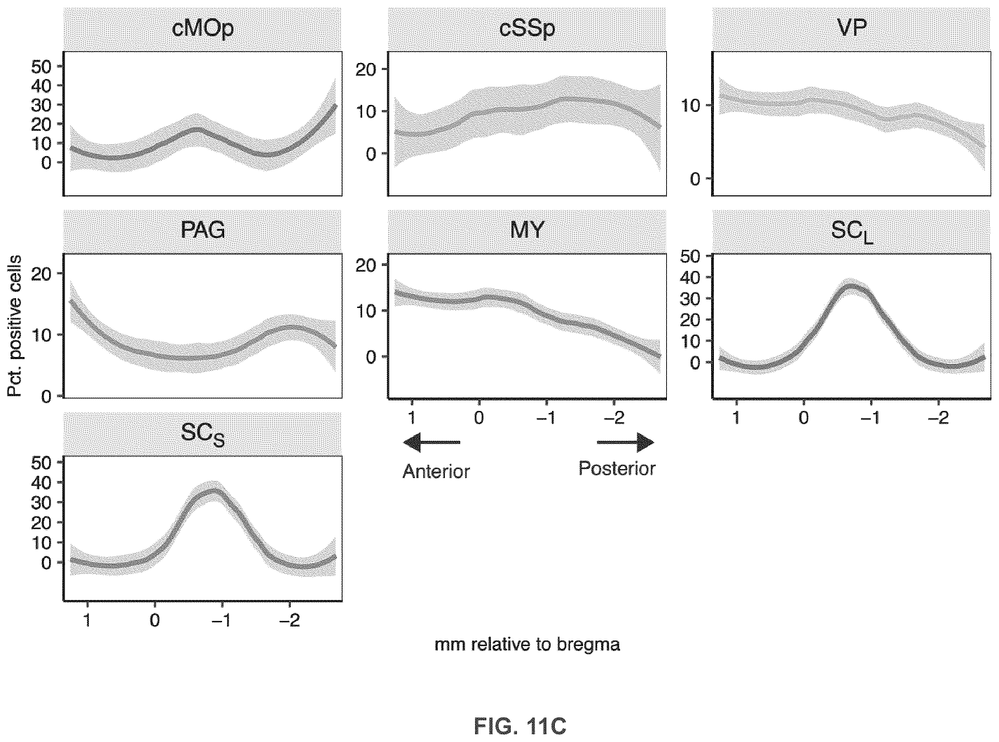

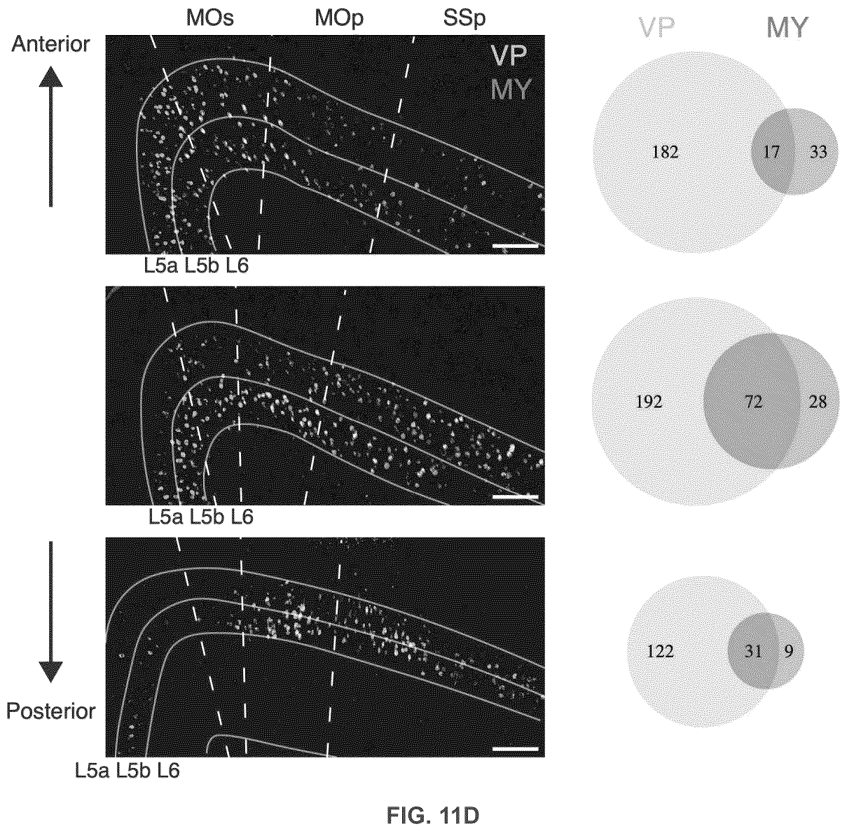

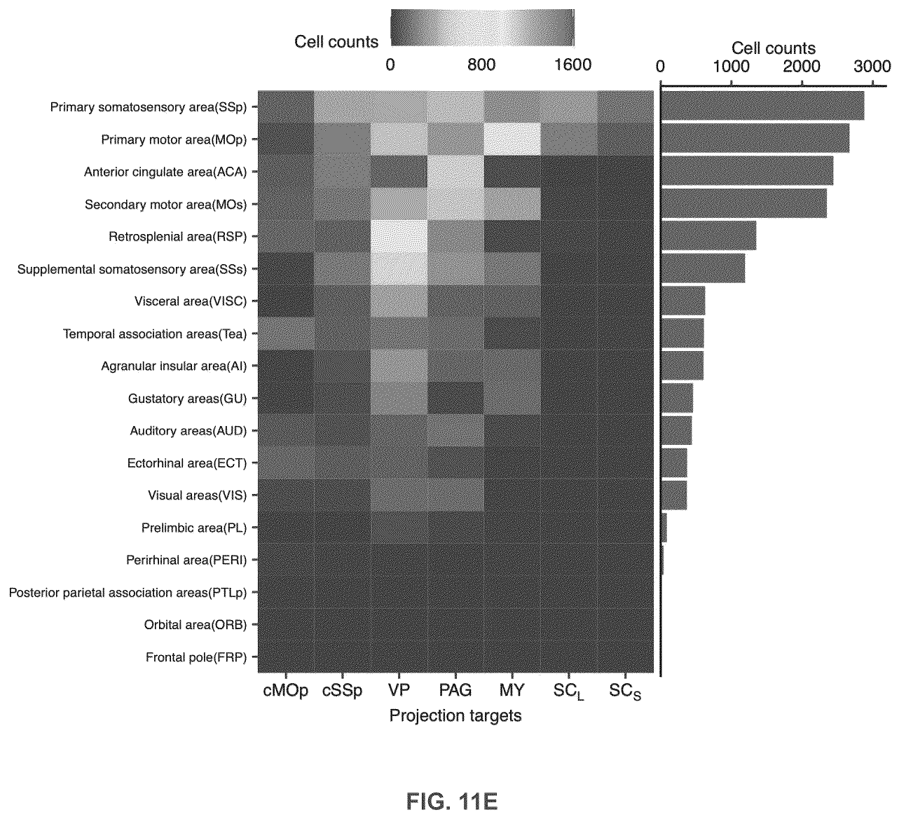

As it has been well established that neurons projecting to IT and ET targets exhibit distinct spatial distribution across the cortex, validation of the spatial distribution of Projection-TAG labeled neurons was performed following AAV injection into the projection targets mentioned above. The distribution of neurons projecting to each target was quantified in several neocortical areas (FIG. 11C). It was observed that neurons projecting to different targets are enriched in distinct areas across the cortex (FIG. 1F, FIG. 1G). For example, cMOp-projecting neurons are most enriched in the ACA, MOs, and the medial part of MOp, whereas cSSp-projecting neurons are dominantly found in MOp and SSp. While VP-, PAG-, and MY-projecting neurons are distributed across multiple cortex areas, SC_{L}- and SC_{S}-projecting neurons are highly enriched around MOp and SSp. The spatial distribution of projection neurons is largely consistent with previous reports. It has been reported that thalamus- and MY-projecting neurons in the anterior lateral motor cortex exhibit distinct spatial distribution in layer 5 (L5). It was similarly observed that VP-projecting neurons, while also found in L6, were more enriched in L5a than L5b (p=0.005), whereas MY-projecting neurons were more abundant in L5b than L5a (p=0.002) in the anterior part of the MOp (FIG. 11D). Interestingly, their distinct sub-layer distribution appeared to attenuate towards the posterior MOp, accompanied by an increased proportion of neurons co-projecting to both targets. While numerous cortex areas contain neurons projecting to the seven targets examined herein, analysis highlights the MOp and the SSp as particularly rich in projection neurons among all cortex areas analyzed and are highly enriched with neurons projecting to each of the seven targets (FIG. 11E).

### A Multi-Modal Single-Cell Atlas of Mouse Cortex

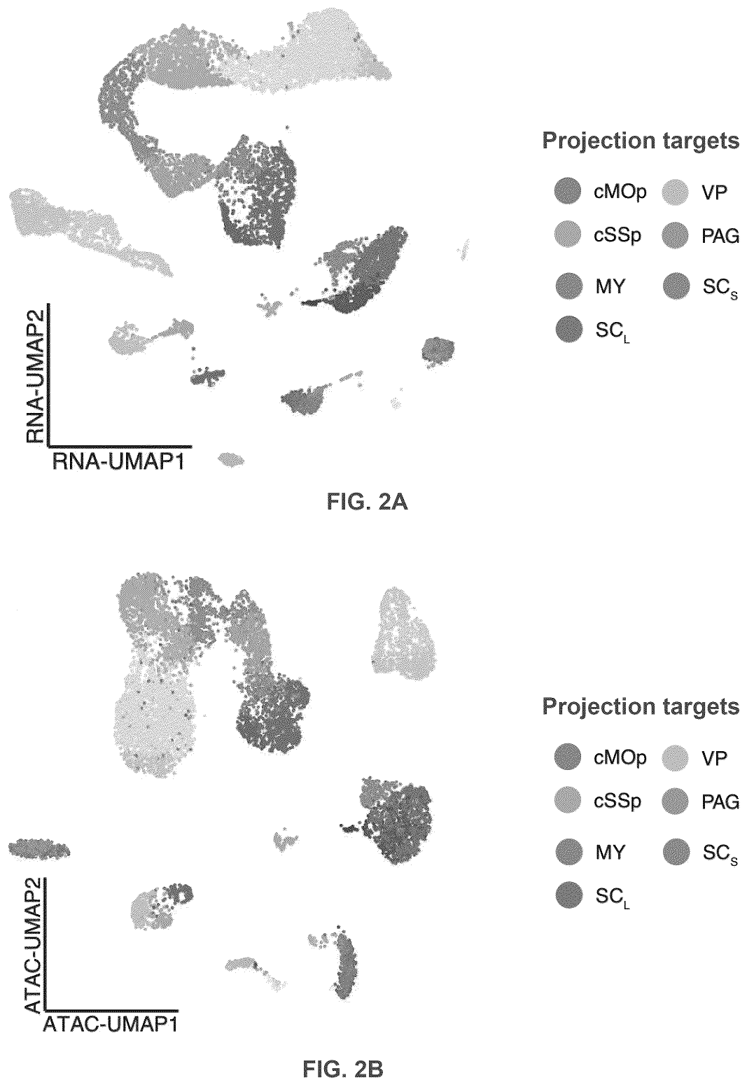

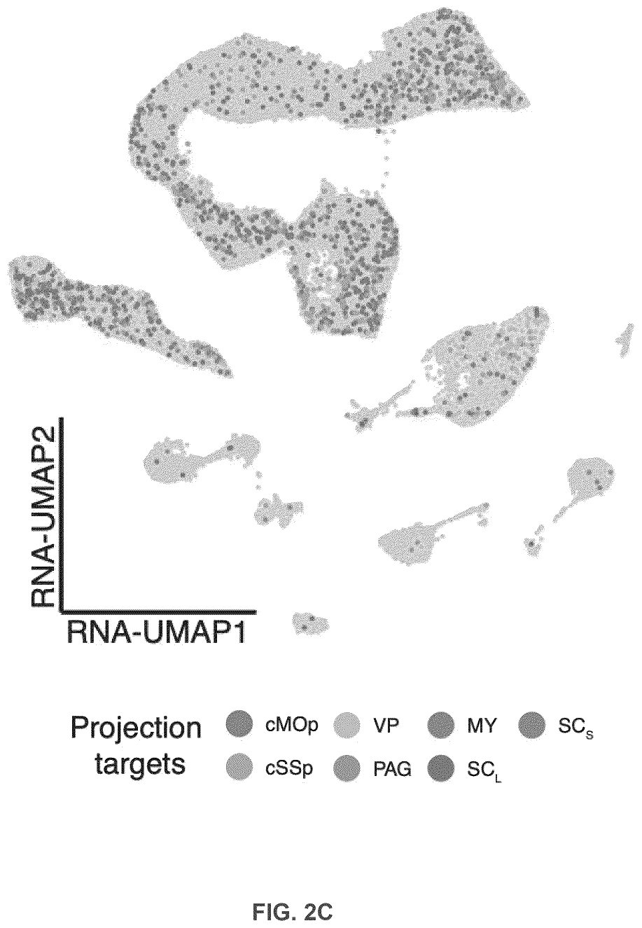

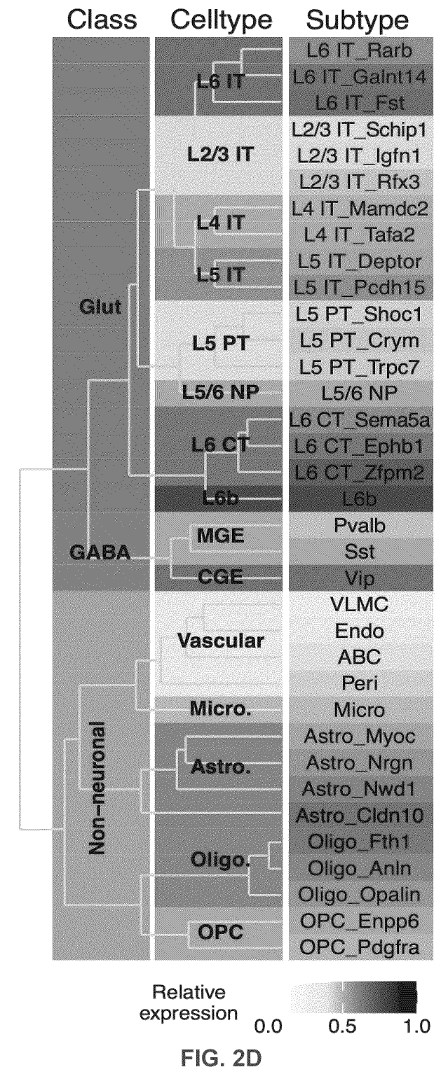

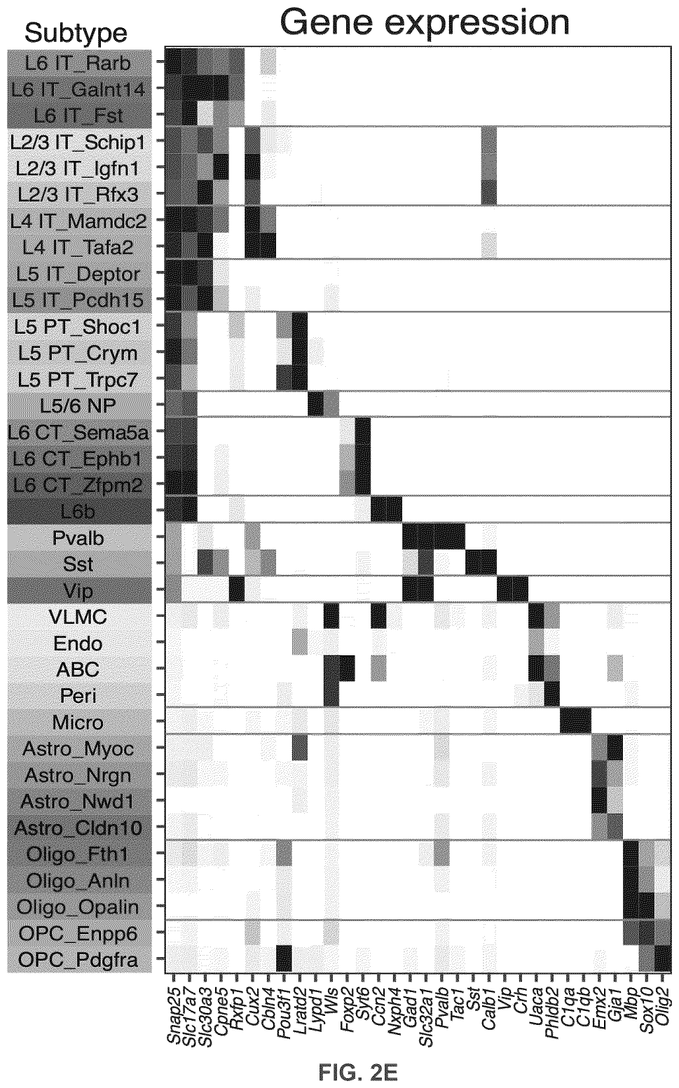

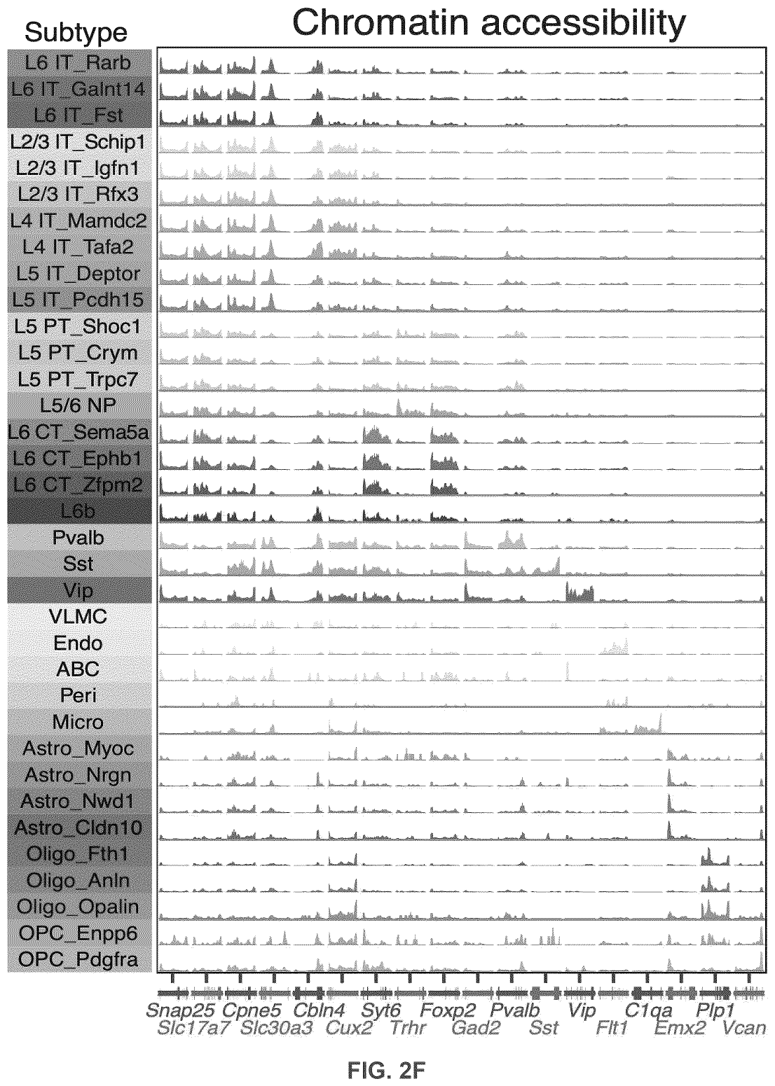

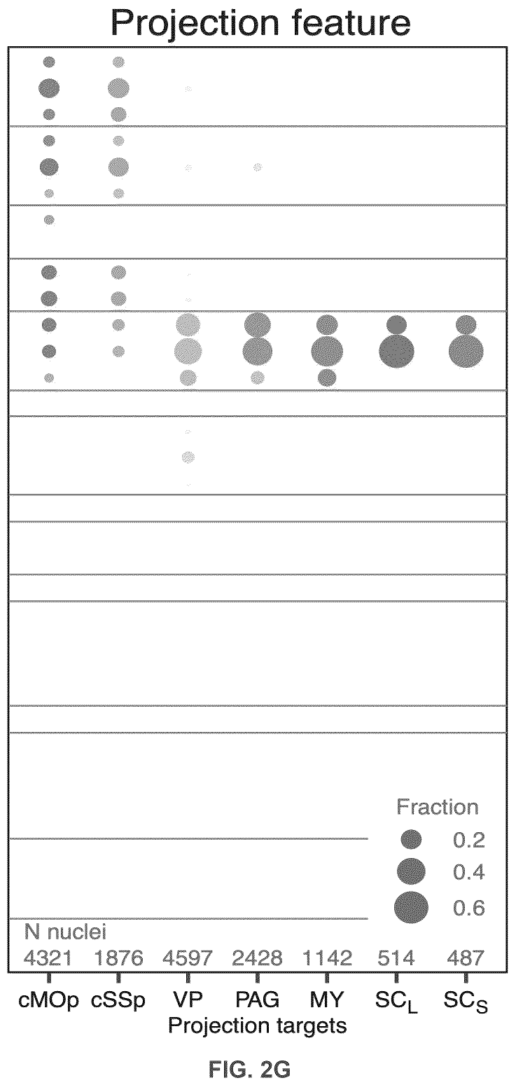

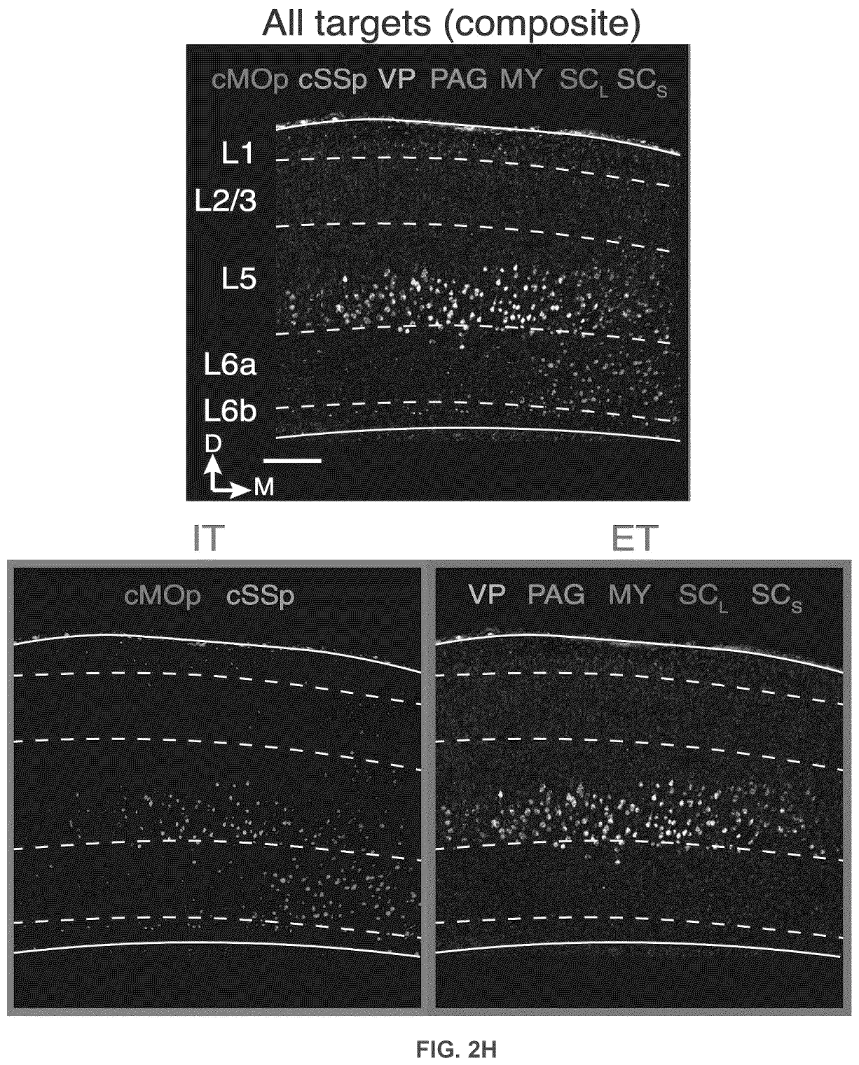

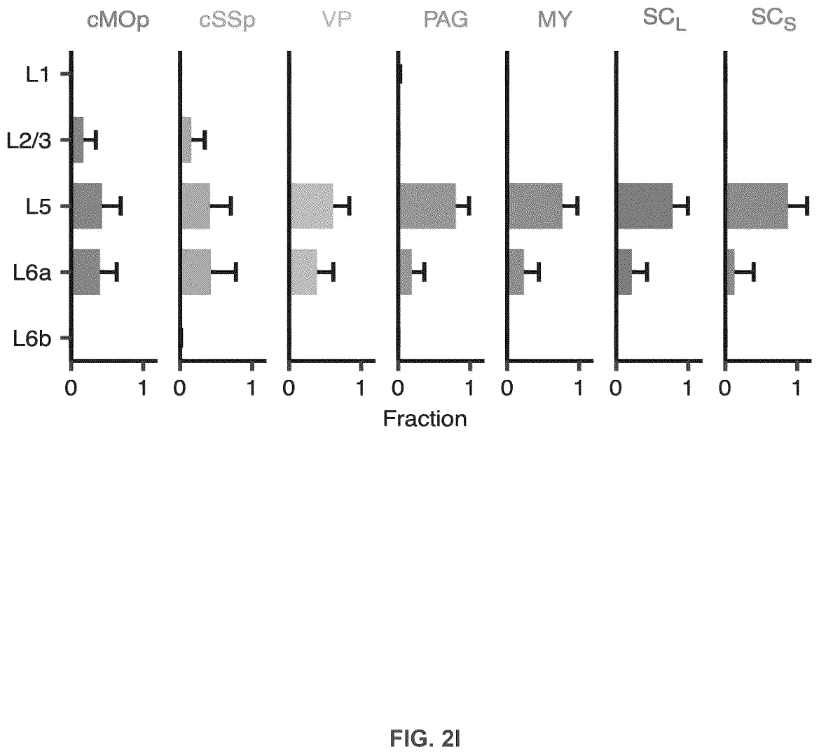

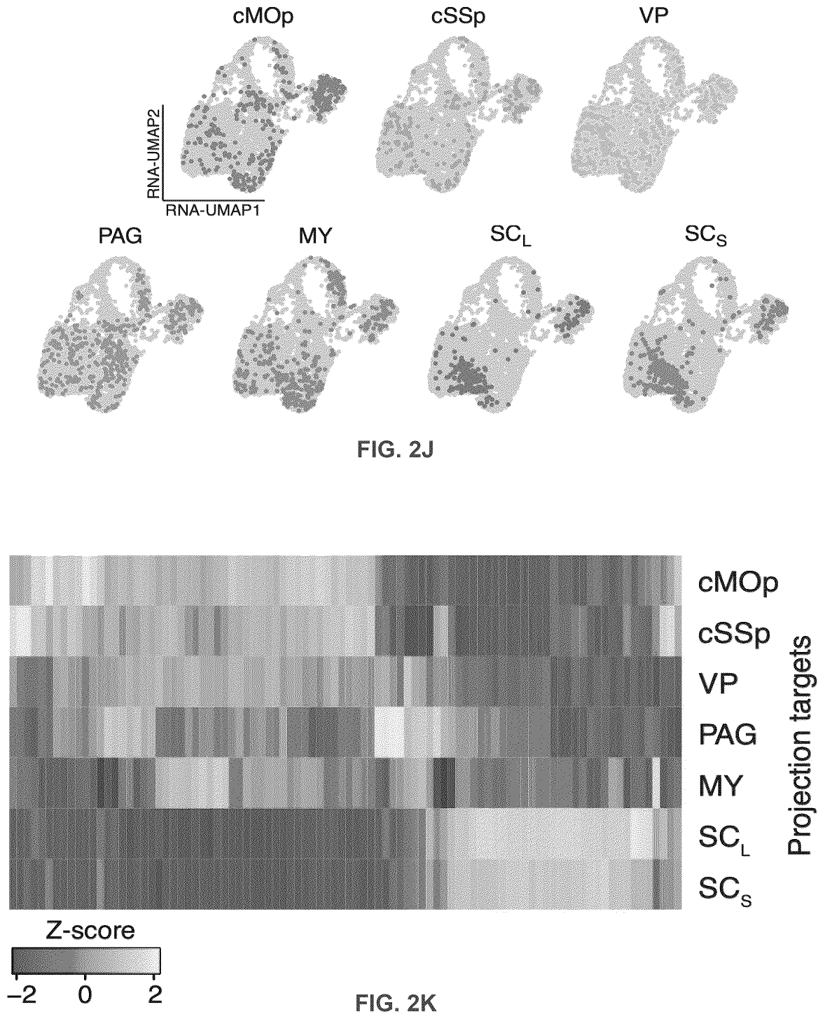

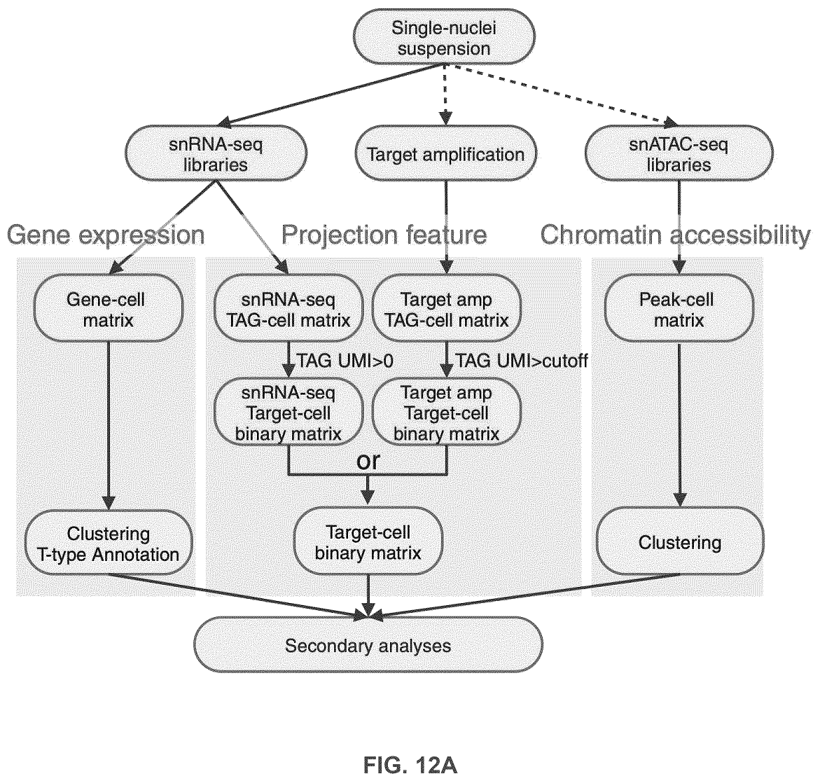

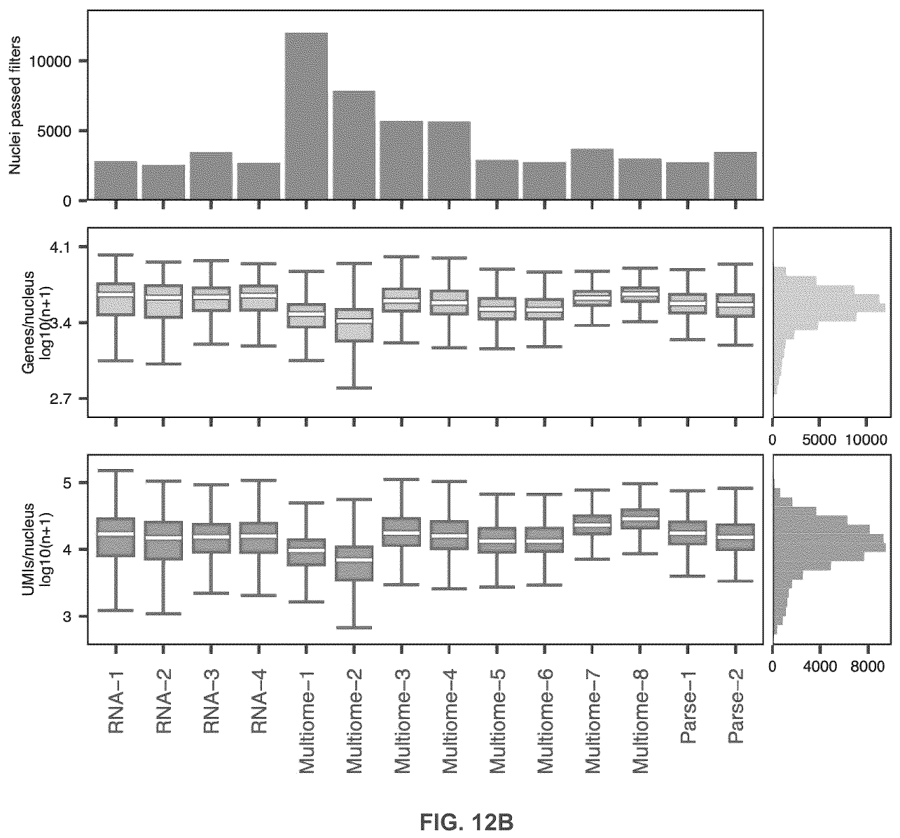

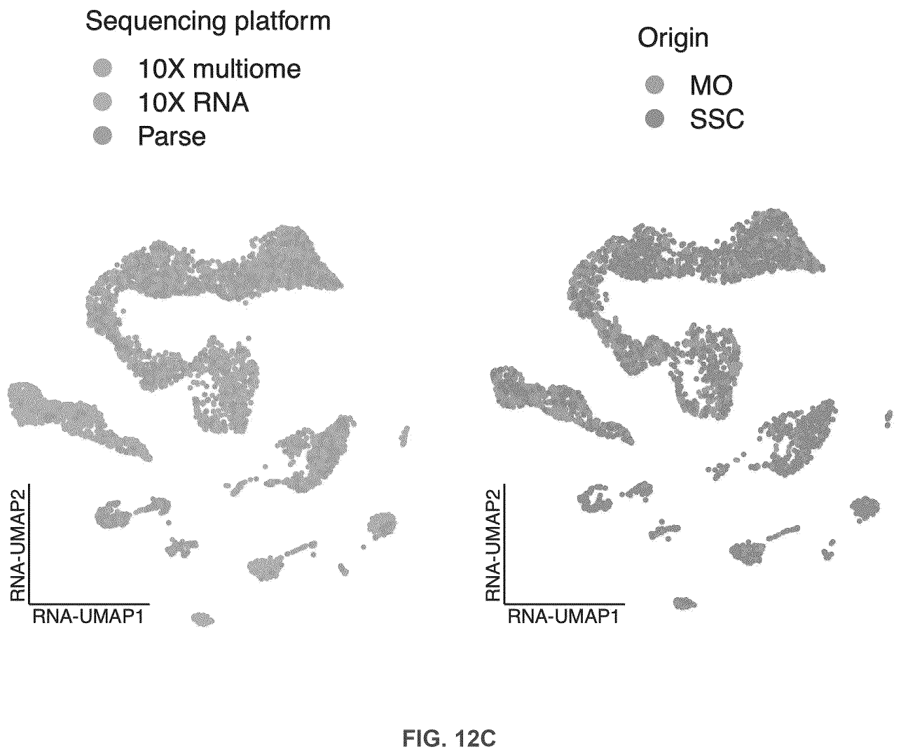

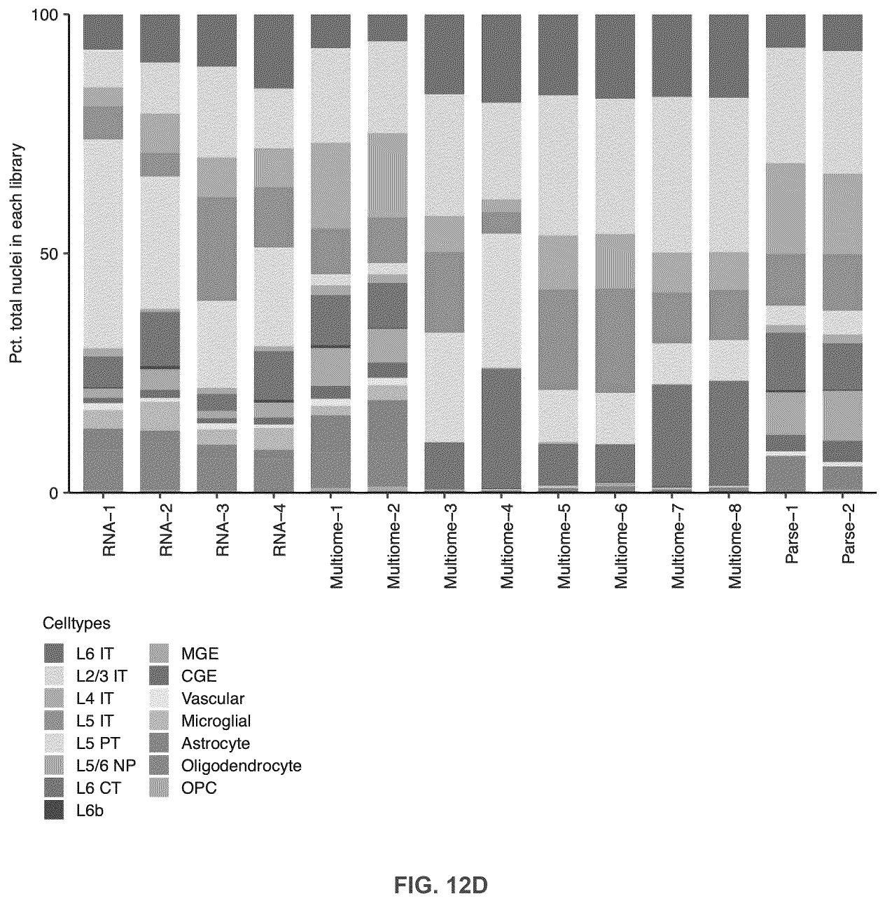

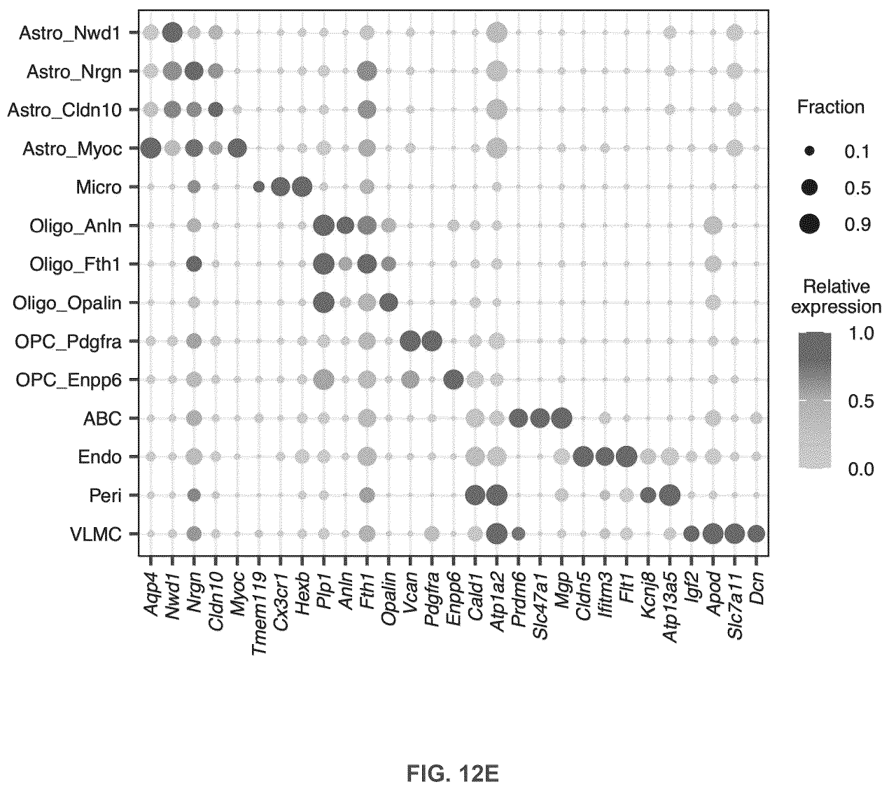

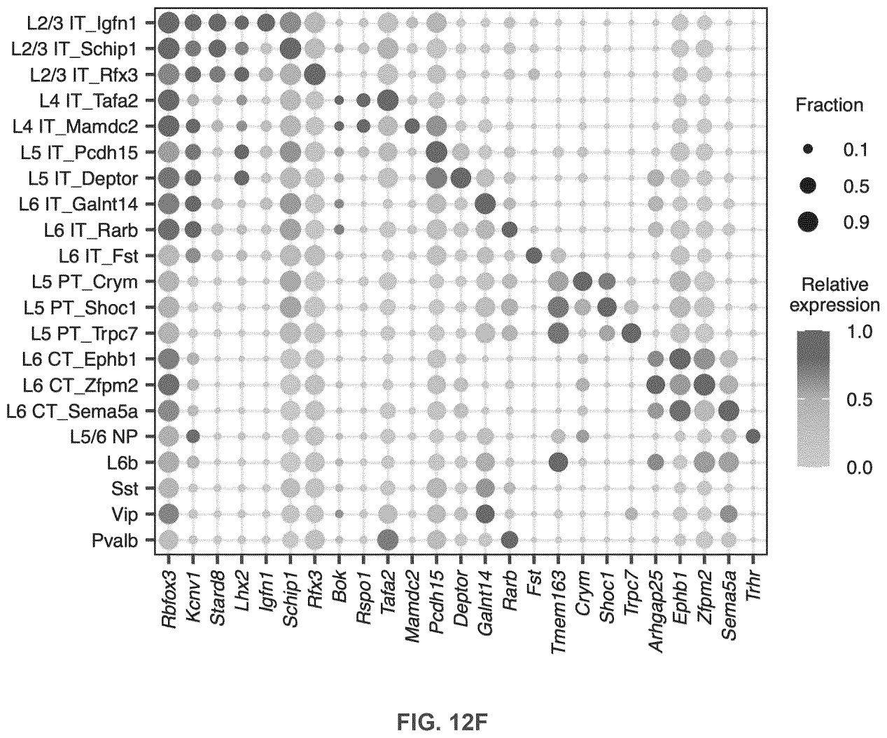

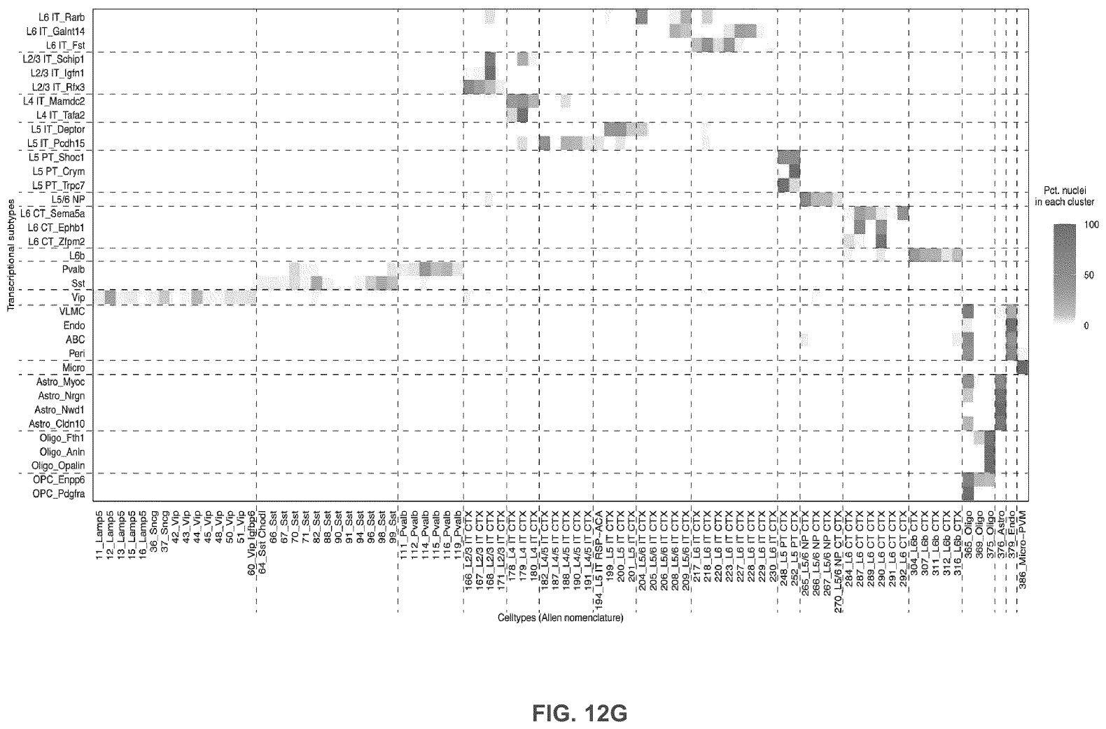

Next, simultaneous profiling was assessed for gene expression, chromatin accessibility, and projection feature of the same cells using Projection-TAGs in multiomic analysis of snRNA-seq and single-nucleus ATAC-seq (snATAC-seq, FIG. 12A). First, snRNA-seq was performed on four MOp samples and six SSp samples from mice receiving injections of Projection-TAG AAVs into seven downstream projection targets mentioned above, generating 14 libraries containing a total of 69,657 nuclei with an average of 3,621 genes per nucleus (FIG. 12B). After quality control and removal of low-quality nuclei and doublets, 61,387 nuclei were retained in the snRNA-seq dataset. Notably, nuclei from individual libraries co-clustered together, indicating a low batch effect across libraries and largely shared gene expression profiles between nuclei from MOp and SSp (FIG. 12C, FIG. 12D). Clustering analysis revealed 35 distinct transcriptional clusters, which were further classified into three major classes (Glutamatergic neurons, GABAergic neurons, and non-neuronal cells, FIG. 2A). Each cluster was assigned to a known cell type, based on the expression of canonical marker genes (FIG. 2D, FIG. 2E). Previous studies revealed that different glutamatergic neuronal types, characterized with different anatomical properties such as layer distributions and projection targets, exhibited distinct gene expression profiles. Following this naming convention, glutamatergic cell types were assigned to 18 glutamatergic neuronal clusters based on their layer enrichment and projection patterns previously described: IT neurons from layers 2/3 (L2/3 IT), 4 (L4 IT), 5 (L5 IT), and 6 (L6 IT), pyramidal tract neurons from layer 5 (L5 PT), near-projecting neurons from layers 5/6 (L5/6 NP), corticothalamic-projecting neurons from layer 6 (L6 CT), and neurons from layer 6b (L6b). Three GABAergic neuronal cell types were defined based on developmental origins: medial ganglionic eminence (MGE) neurons and caudal ganglionic eminence (CGE) neurons. Five non-neuronal cell types were also identified: vascular cells, microglial cells (Micro.), astrocytes (Astro.), oligodendrocytes (Oligo.), and oligodendrocyte progenitor cells (OPC). Subtypes were assigned to each cluster based on the marker gene distinctly expressed in that cluster compared to other clusters of the same cell type (FIG. 12E, FIG. 12F). While some variability of cell type distribution was observed across libraries, likely due to different nuclear preparation approaches (FIG. 12D), the gene expression profiles of individual subtypes are largely consistent with previous reports (FIG. 12G).

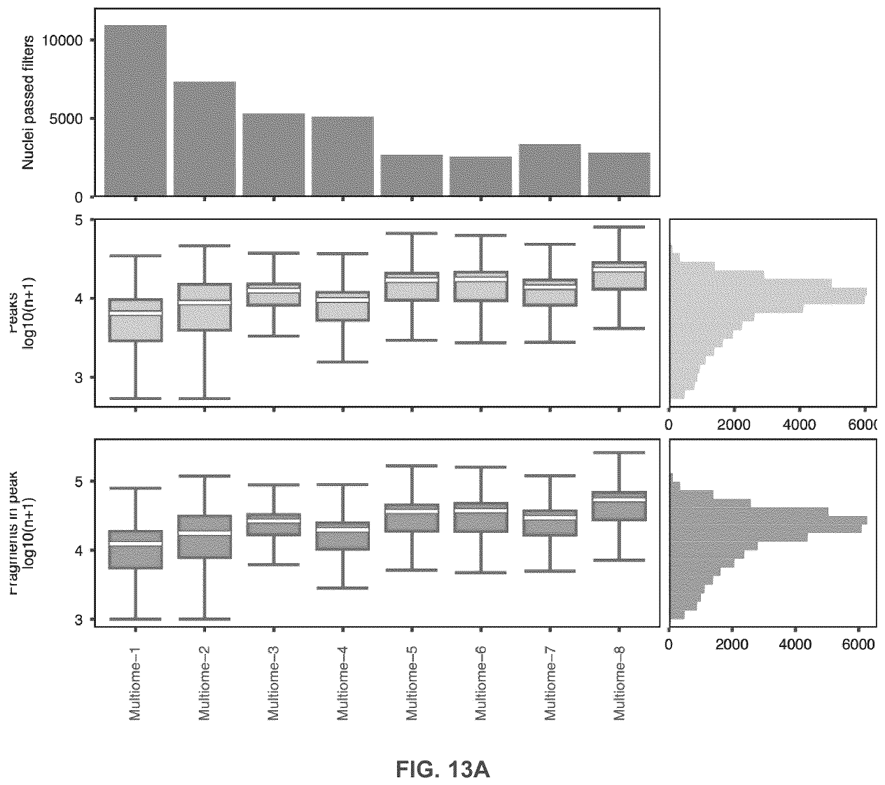

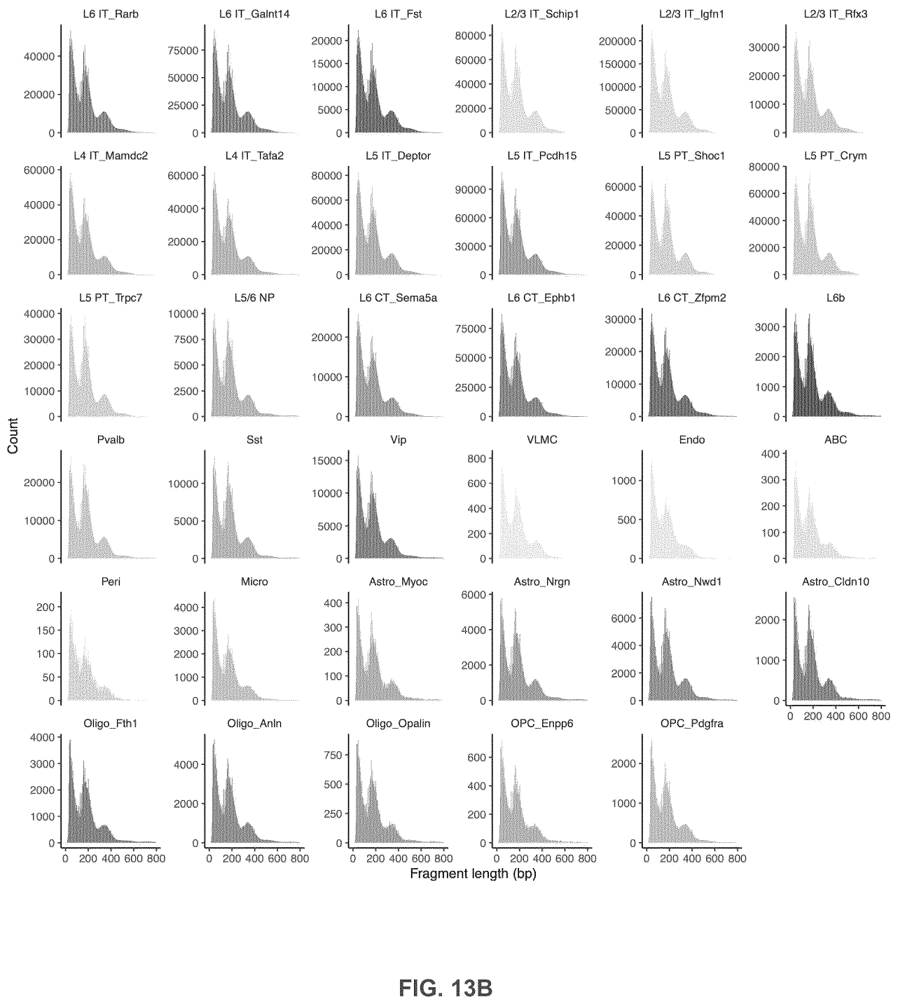

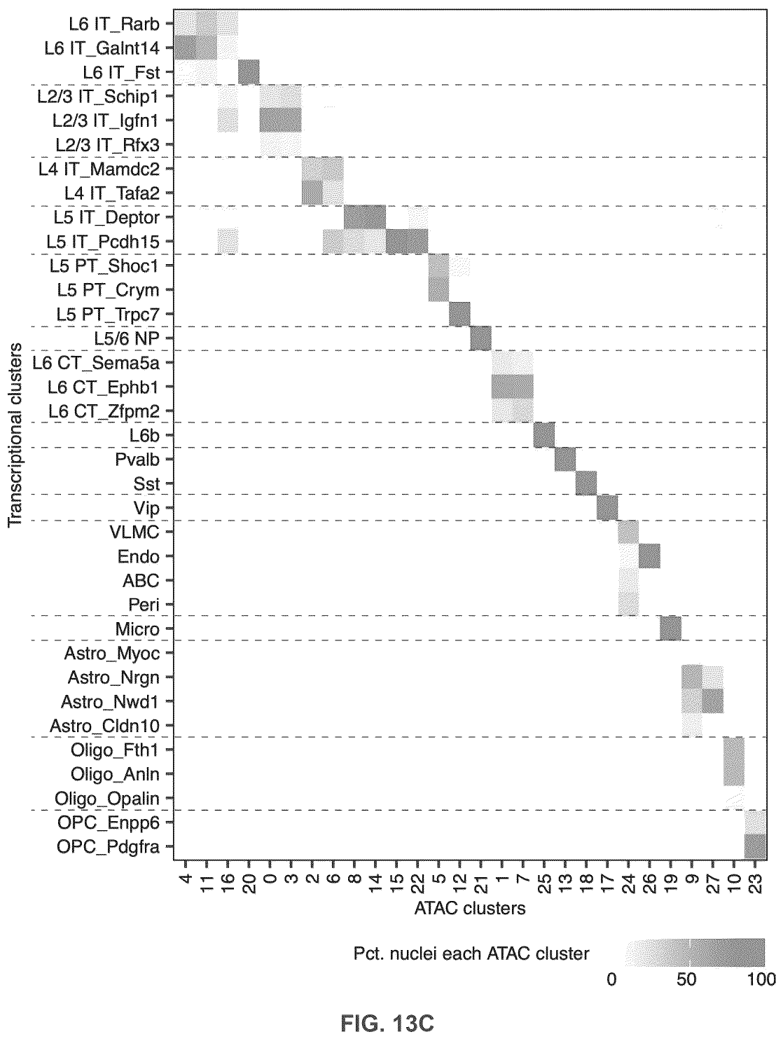

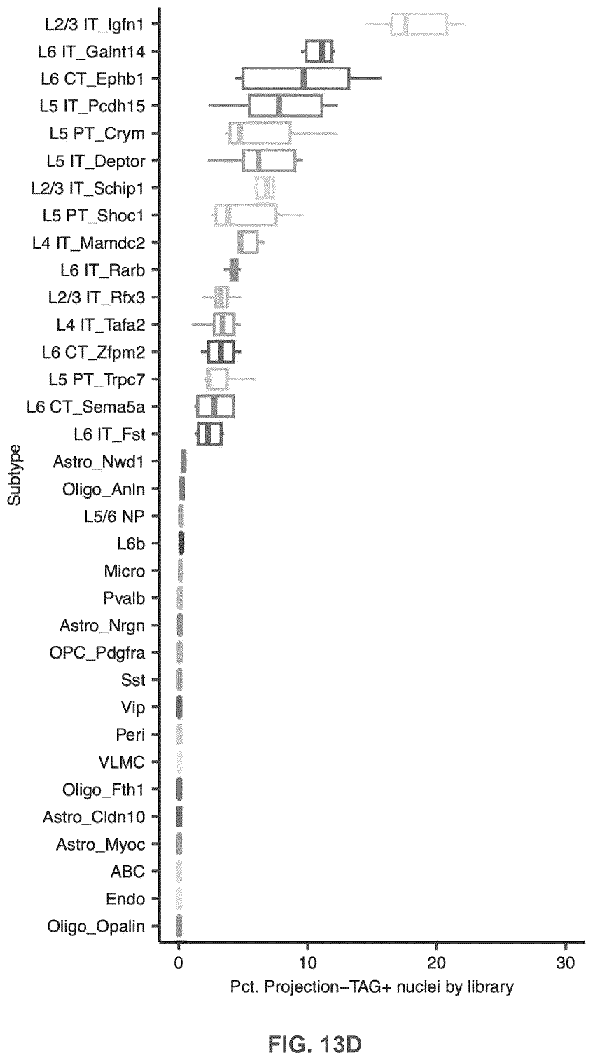

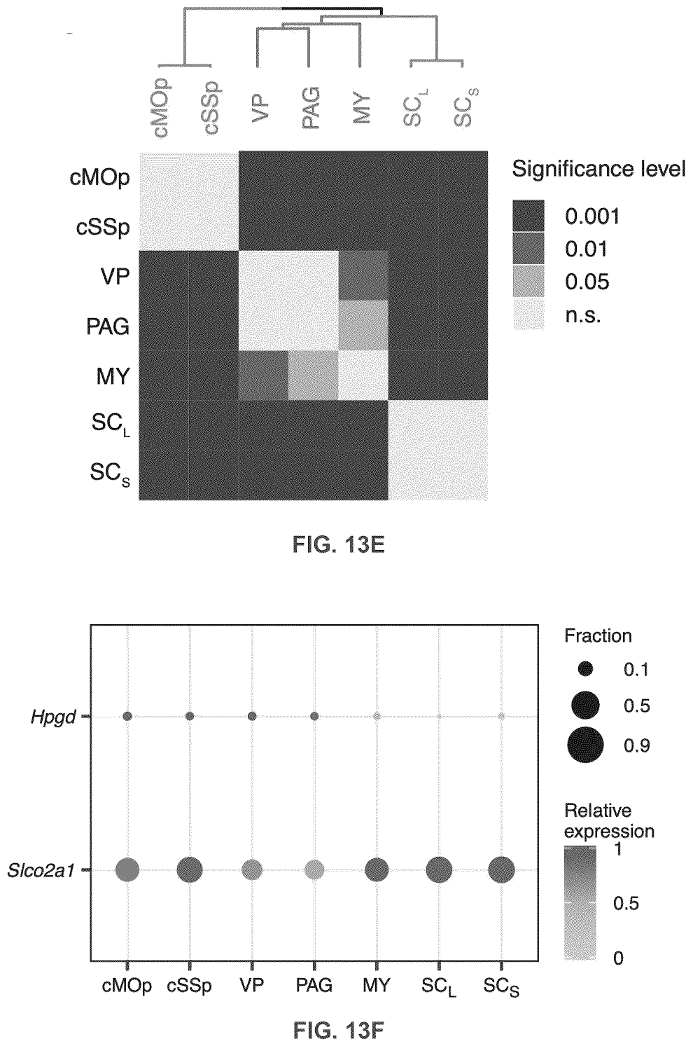

To enable simultaneous investigation of the gene expression and chromatin accessibility profiles in the same cells, combinatorial snATAC-seq was performed on eight snRNA-seq libraries described herein above, generating a dataset of 40,188 high-quality nuclei with an average sequencing depth of 25,412 transposase-sensitive fragments per nucleus (FIG. 13A). These snATAC-seq fragments captured the open chromatin regions of the genome as they followed the expected nucleosomal size distribution (FIG. 13B). Clustering analysis of the snATAC-seq data revealed 28 distinct clusters. The chromatin accessibility and gene expression profiles of the sequenced nuclei are highly correlated, with 87.7±18.0% nuclei in each snATAC-seq cluster assigned to the same transcriptional clusters as those made when independently analyzed for snRNA-seq data (FIG. 2B, FIG. 12C). In addition, nuclei in individual snATAC-seq clusters display distinct chromatin accessibility around the genomic loci of canonical marker genes for the corresponding transcriptional subtypes (FIG. 2F). Consequently, snATAC-seq nuclei were grouped by transcriptionally defined cell types and subtypes in subsequent analysis.

Detection and demultiplexing of Projection-TAGs by snRNA-seq enabled investigation of the projection feature of individual sequenced cells, thus allowing multi-modal profiling of projection neurons at single-cell resolution (FIG. 2C). It was determined whether a snRNA-seq nucleus project to each of the seven targets based on its expression of the corresponding TAG. To correlate the transcriptional identity with projection targets, the subtype composition of nuclei that are positive for each TAG was first examined (FIG. 2F, FIG. 13D). In this analysis, a nucleus positive for the corresponding Projection-TAG will be included for the analysis of a projection target, regardless of the expression of other Projection-TAGs in the same nucleus. Hierarchical clustering based on the subtype composition for each project target divided the seven targets into three clusters (FIG. 13D). The first cluster contains two IT targets, cMOp and cSSp. They both consist of transcriptionally-defined L2/3 IT, L4 IT, L5 IT, L6 IT, and L5 PT neurons and their subtype composition are not significantly different from each other (23.2% and 27% L2/3 IT, 4.9% and 4% L4 IT, 20.6% and 18.4% L5 IT, 29.9% and 35.8% L6 IT, 19% and 12.5% L5 PT, MOp and SSp, respectively). The second cluster contains three ET/subcortical targets: VP, PAG, and MY. While they are all enriched of L5 PT neurons (78.1% VP, 85.4% PAG, and 86.6% MY), MY-projecting neurons have proportionally fewer nuclei from L5 PT_Shoc1 cluster (20.7%, compared to 28.3% VP and 33.7% PAG). In addition, 10.4% of the VP-projecting neurons are from transcriptionally defined L6 CT subtypes. The third cluster contains two ET/corticospinal targets with SC_{L} and SC_{S} projecting neurons are enriched of L5 PT subtypes but are underrepresented in L5 PT_Trpc7 subtype (3.9% each) compared to other ET projections (subcortical: VP, PAG, and MY). Spatial analysis of multiplexed FISH further confirmed their layer distribution in MOp (FIG. 2H, FIG. 2I). The cell types of projection neurons are consistent with the explicit correspondence between neurons projecting to IT and ET targets previously described.

As transcriptionally defined L5 PT neurons are enriched of nuclei positive for Projection-TAGs of all seven projection targets, it was determined whether L5 PT neurons projecting to different targets are transcriptionally distinct. Subclustering of 4,168 L5 PT Projection-TAG+ nuclei present in both snRNA-seq and snATAC-seq data revealed distinct transcriptional and epigenetic profiles of L5 PT neurons projecting to each target (FIG. 2J, FIG. 2K). Hpgd and Slco2a1 have been reported as marker genes for thalamus- and MY-projecting neurons, respectively. Interestingly, an expression gradient was observed of those genes across L5 PT neurons projecting to different targets (FIG. 13F), which appears to correlate to the distance of the projections, raising the possibility that the projection targets of neurons may be dictated/maintained by a shared, fine-tuned transcriptional program.

### Projection-TAGs Revealed Axonal Collaterals and Complex Wiring Diagram

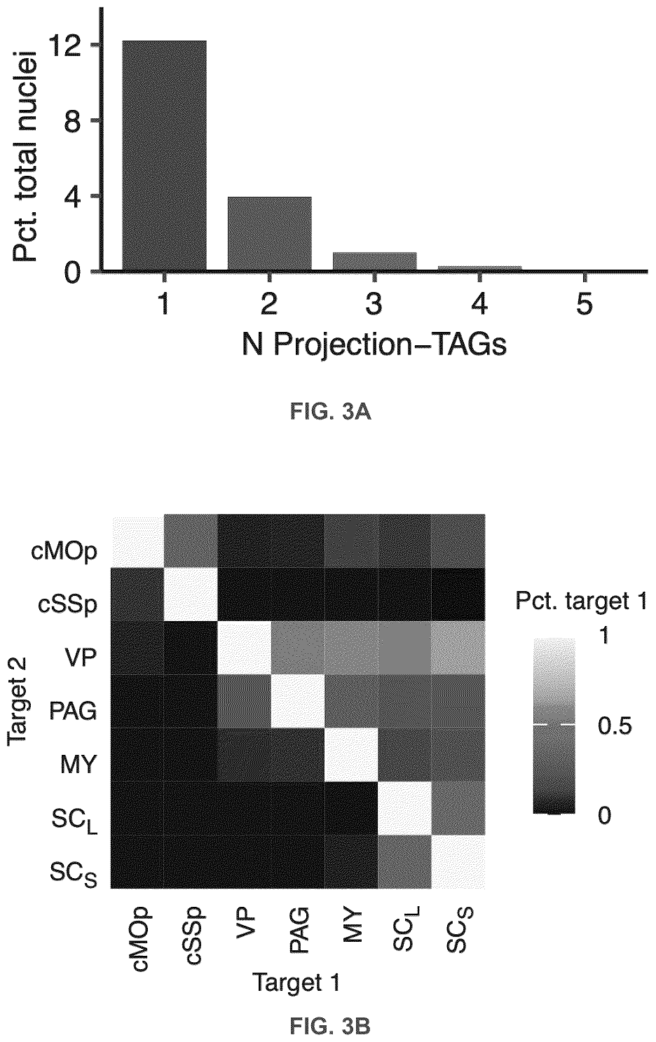

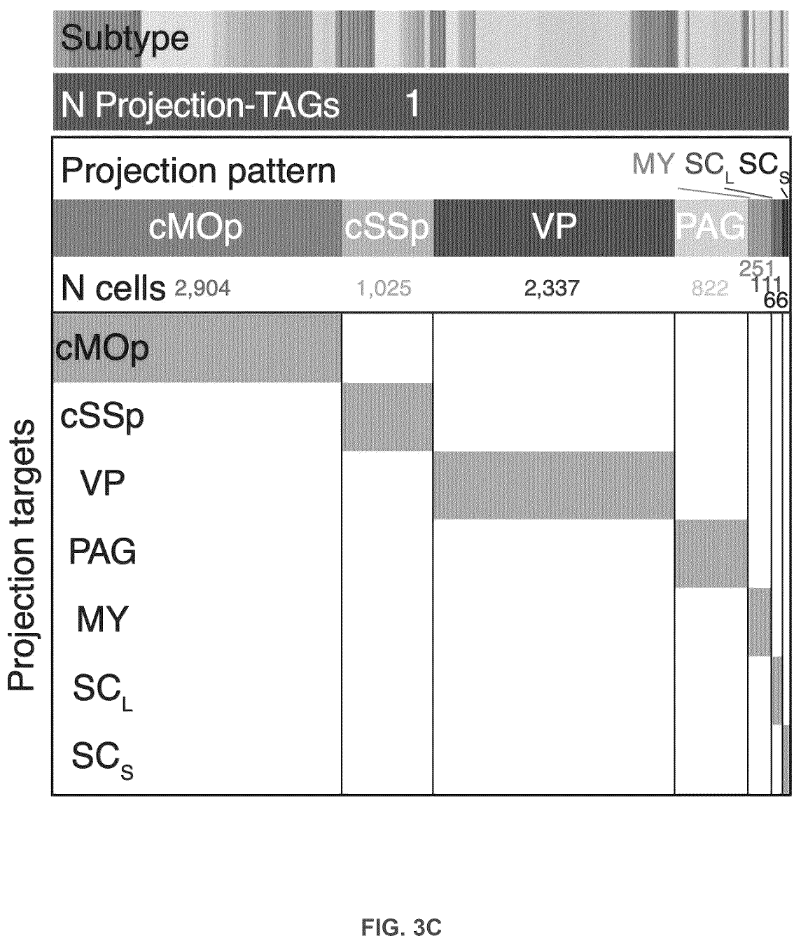

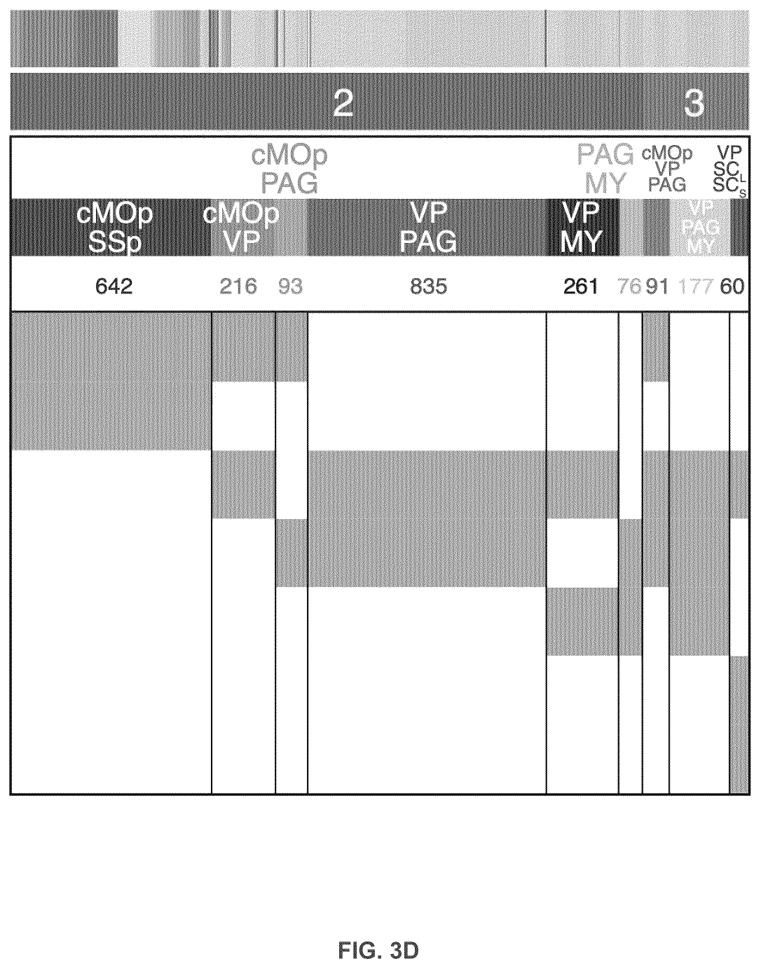

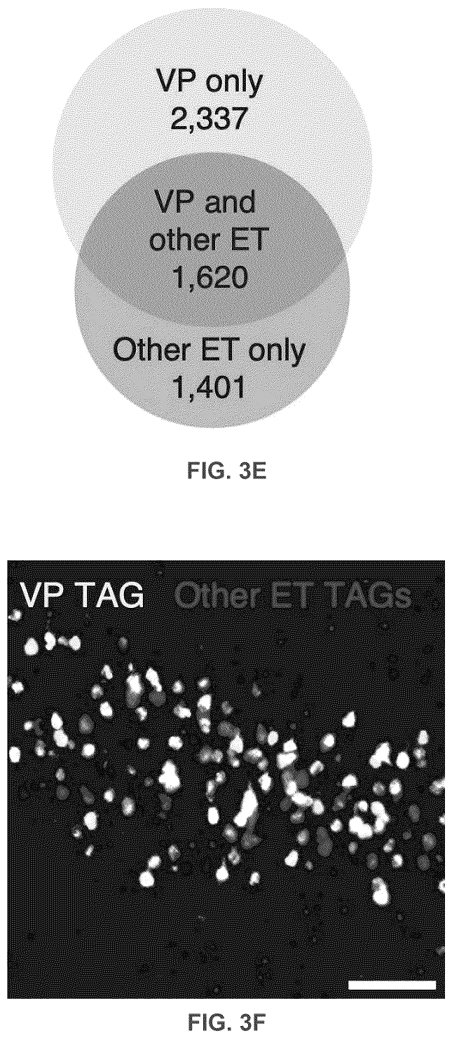

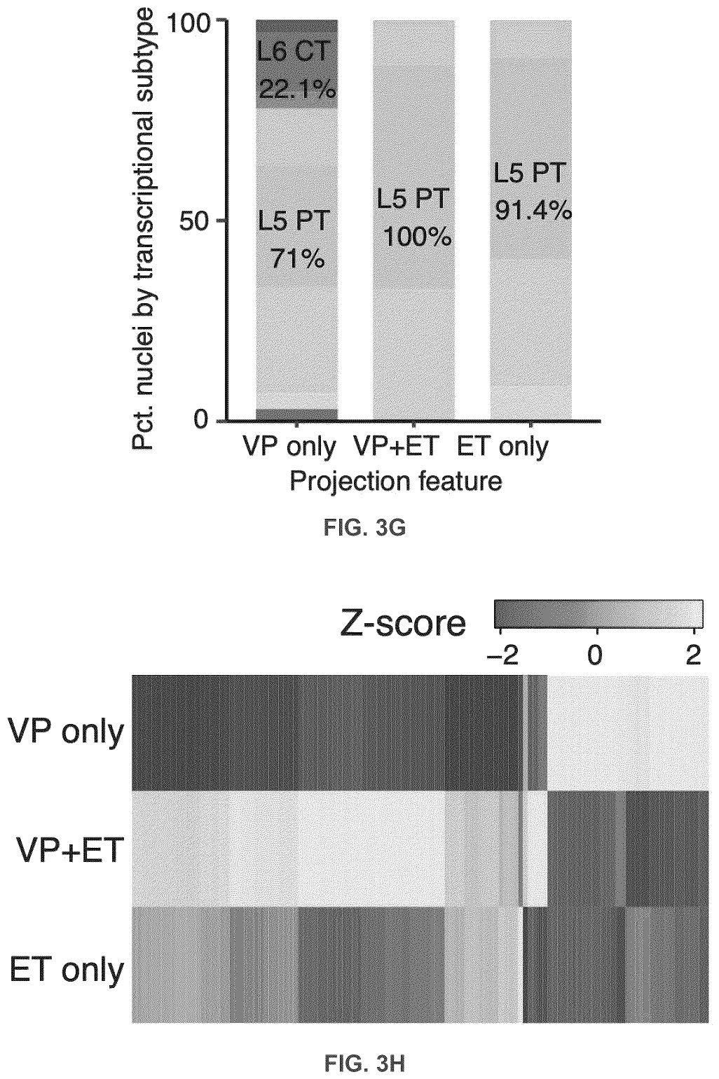

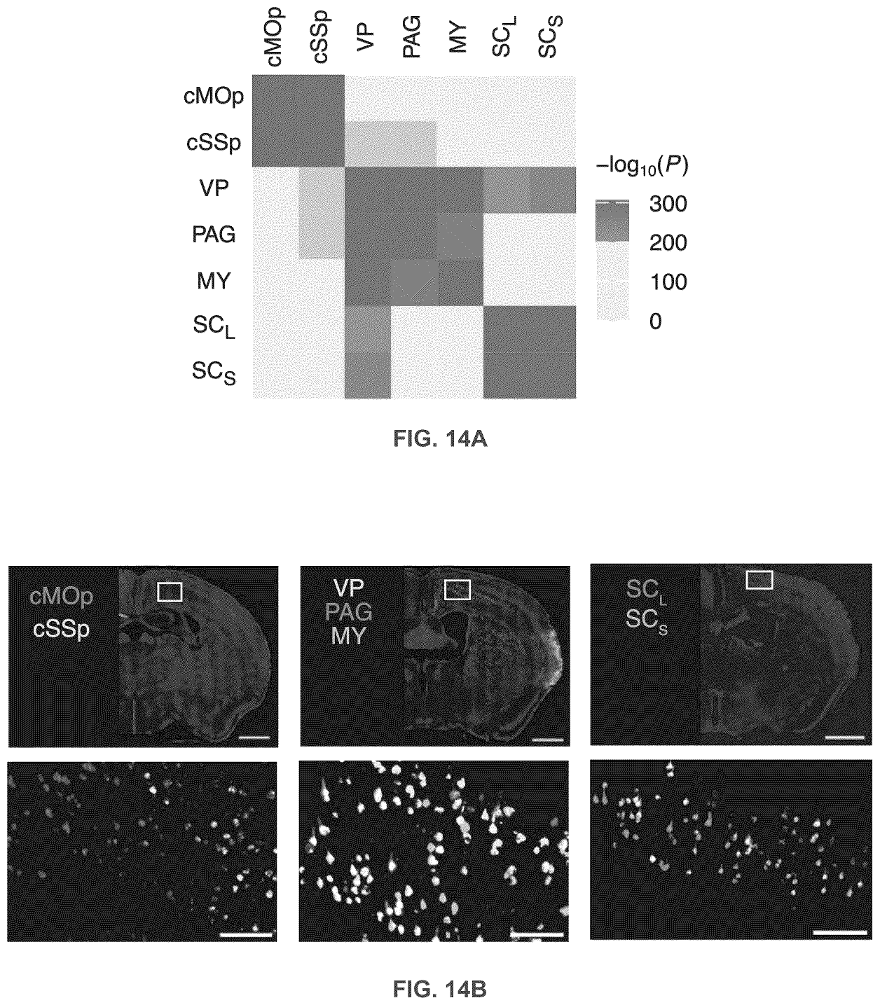

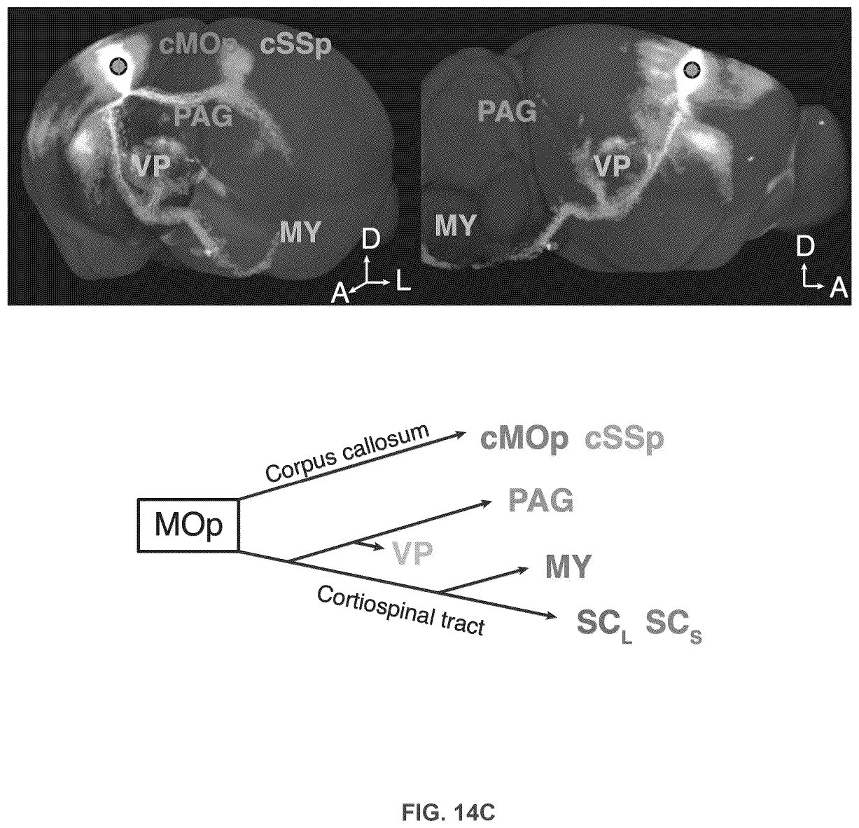

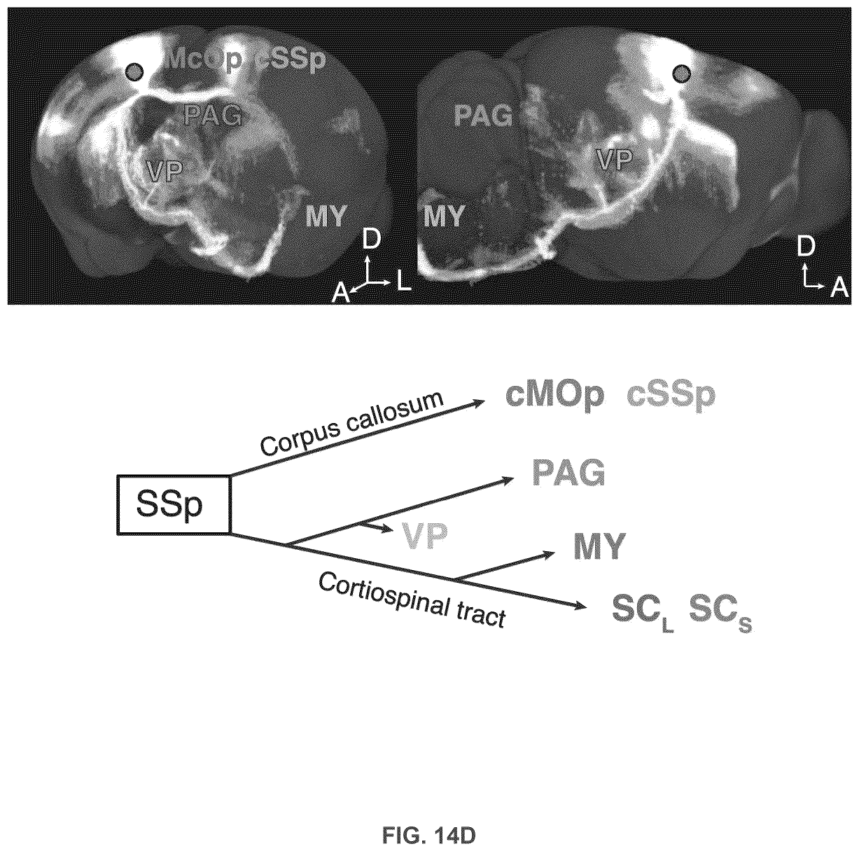

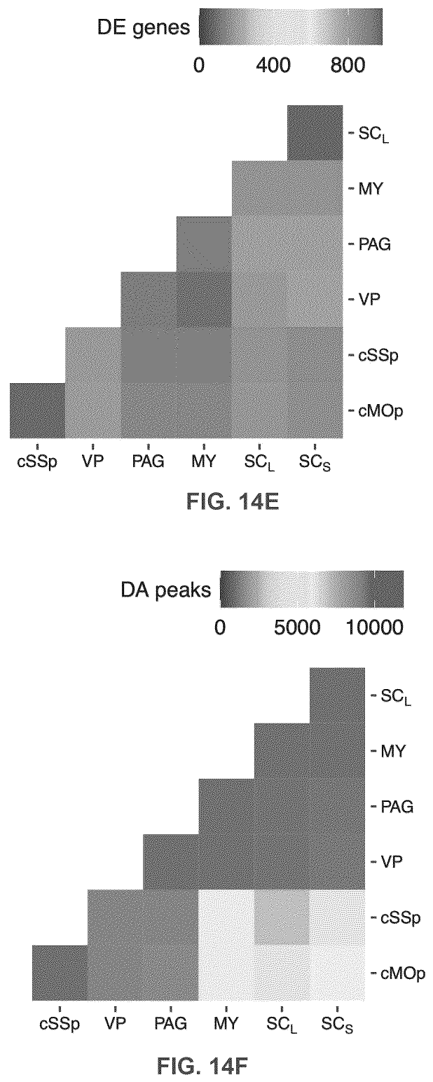

Brain regions are interconnected with complex wiring diagrams. If a brain region projects to N downstream targets, the possible projection pattern of a single neuron will have up to 2^{N} combinations. As Projection-TAGs enable multiplex projection tracing in single animals, it opens new possibilities for studying neuronal collaterals to multiple targets. A neuron with its axonal collaterals terminated in multiple targets may be labeled by multiple Projection-TAGs. Indeed, 30.6% of Projection-TAG+ snRNA-seq nuclei express multiple Projection-TAGs (FIG. 3A). To investigate the overall pattern of axonal collaterals, the overlap of neurons projecting to any two targets was first calculated (FIG. 3B). Two IT targets (cMOp and cSSp) exhibited highly significant overlap (FIG. 14A) with each other with 16.8% MOp-TAG+ nuclei or 36.8% SSp-TAG+ nuclei are co-labeled by both IT-TAGs. ET targets also showed highly significant overlap with each other, with the most notable overlapping to VP. 56.3-66.7% nuclei labeled with other ET-TAGs (PAG, MY, SC_{L}, and SC_{S}) are also co-labeled with VP-TAG. Additionally, PAG and MY also highly significantly overlap, as well as SC_{L} and SC_{S}. To elucidate the complex projection feature of single neurons, any possible combinations of projection to the seven targets were next investigated. Out of all 128 (2\hat{ }7) possible projection patterns, 16 neuronal populations were identified with distinct projection patterns that passed the FDR cutoff: seven populations with single projections (positive for only one Projection-TAG) (FIG. 3C) and nine populations with multiple projections to two or three of the targets (positive for the Projection-TAGs of the corresponding projection targets and negative for all other Projection-TAGs) (FIG. 3D). The axonal collaterals were further confirmed by multiplexed FISH (FIG. 14B). These observations are highly correlated with the anatomical hierarchy of axonal projections of the cortex previously demonstrated by anterograde bulk tracing techniques (FIG. 14C, FIG. 14D). Moreover, the hierarchical organization of projections is also reflected in the gene expression and chromatin accessibility profiles of neurons projecting to individual targets (FIG. 14E, FIG. 14F). This complementary analysis provides a multi-modal and high-resolution view of the spatial and molecular intricacies of neuronal projections in the mouse brain.

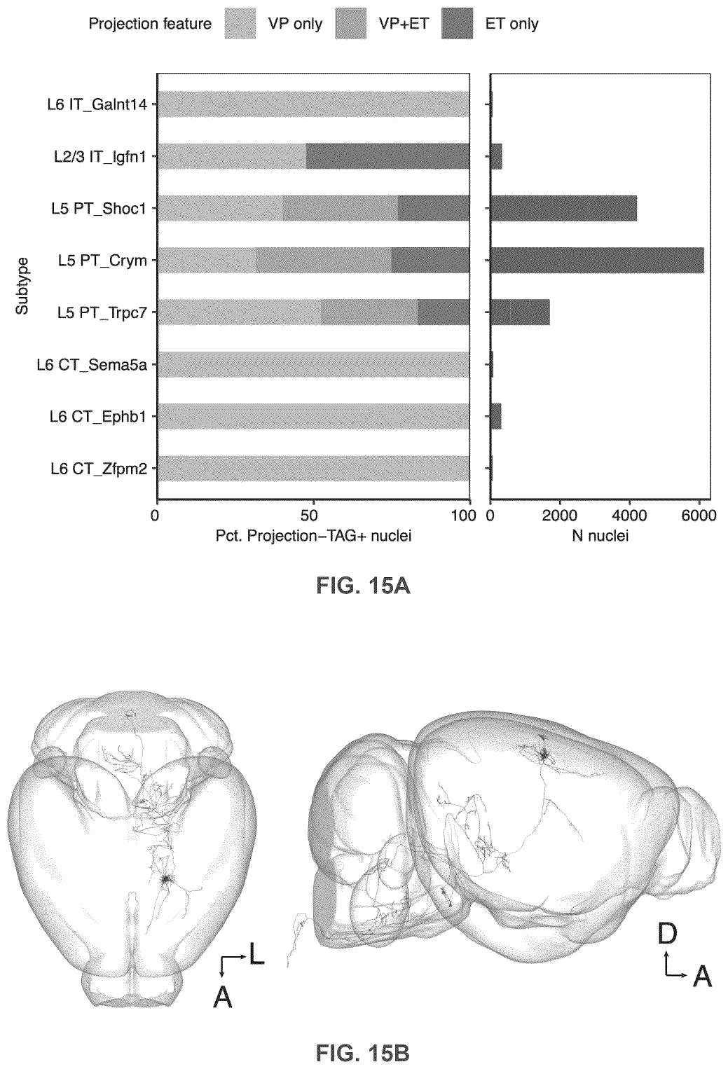

Among 5,358 snRNA-seq nuclei positive for only ET-TAGs, 43.6% and 26.1% express only VP-TAG and only other ET-TAGs (PAG, MY, SC_{L}, and SC_{S}), respectively, and 30.3% express both VP- and other ET-TAGs (FIG. 3E, FIG. 3F). Transcriptionally defined CT neurons restrict their projection to only VP, while transcriptionally defined PT neurons broadly project to various ET targets (FIG. 3G). Observations of axonal collaterals corroborate with the intricate single-neuron projection patterns reported in the MouseLight dataset (FIG. 15A, FIG. 15B) and further demonstrate the complexity of single-neuron projections, which has been overlooked and warrants further investigation. To uncover transcriptional programs that fine-tune cortical projection patterns, 1,971 differentially expressed (DE) genes and 2,737 differentially accessible (DA) peaks in neurons with single projections were identified compared to those with multiple projections (FIG. 3H).

### Characterization of Genomic Cis-Regulatory Elements and Regulated Genes Using Projection-TAGs

While the gene expression program in the diverse MOp and SSp cell types has been largely elucidated by scRNA-seq studies, the regulatory networks that govern the distinct gene expression pattern in individual cell types and projections are under investigated. The genomic regulatory elements (GREs), mostly identified within the non-coding region of the genome and act in a cell-type-specific and tissue-specific manner, fine tune the expression level of the regulated genes. Among different types of GREs, enhancers initiate the recruitment of transcription complexes and drive the transcription of regulated genes while the silencers prevent the expression of regulated genes. Identification of cell-type-specific GREs, specifically enhancers, has gained increased attention in the neuroscience field as they can be used as a valuable tool to mediate transgene (e.g eYFP, ChR2, DREADDS, etc) expression in the target cell populations for basic science research and development of novel therapeutics. Though recent progress has been made in characterizing the landscape of GREs for the projection neurons, the current experimental workflow is tedious and pain-staking as individual projection neurons are labeled via injection of retro Cre in floxed nuclear reporter mice, and regions of interest are pooled for analysis of potential GREs. It was tested whether Projection-TAGs could be used to perform high throughput analysis of identifying projection-specific GREs.

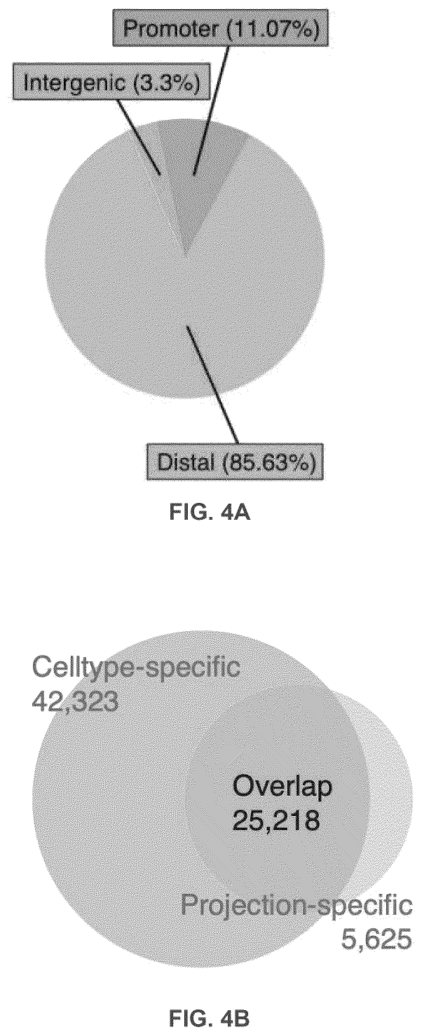

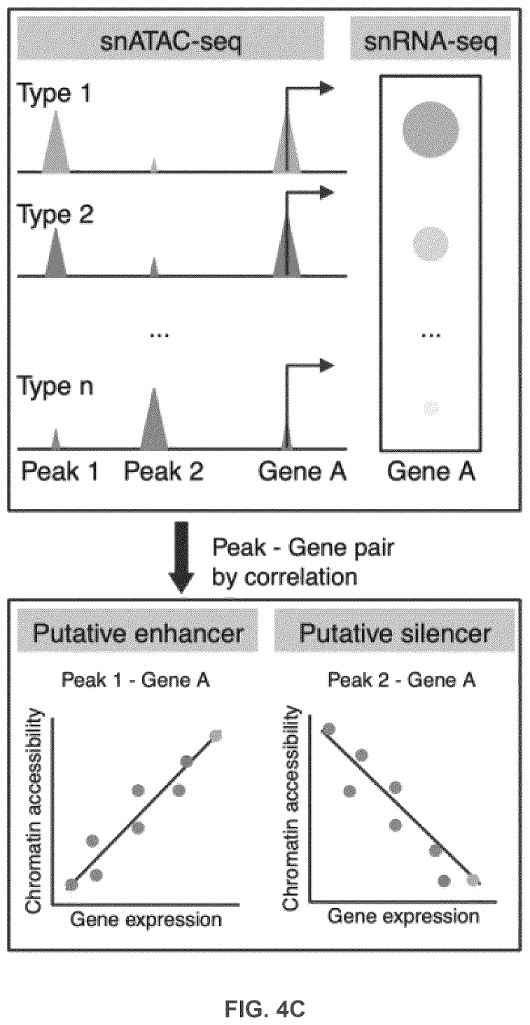

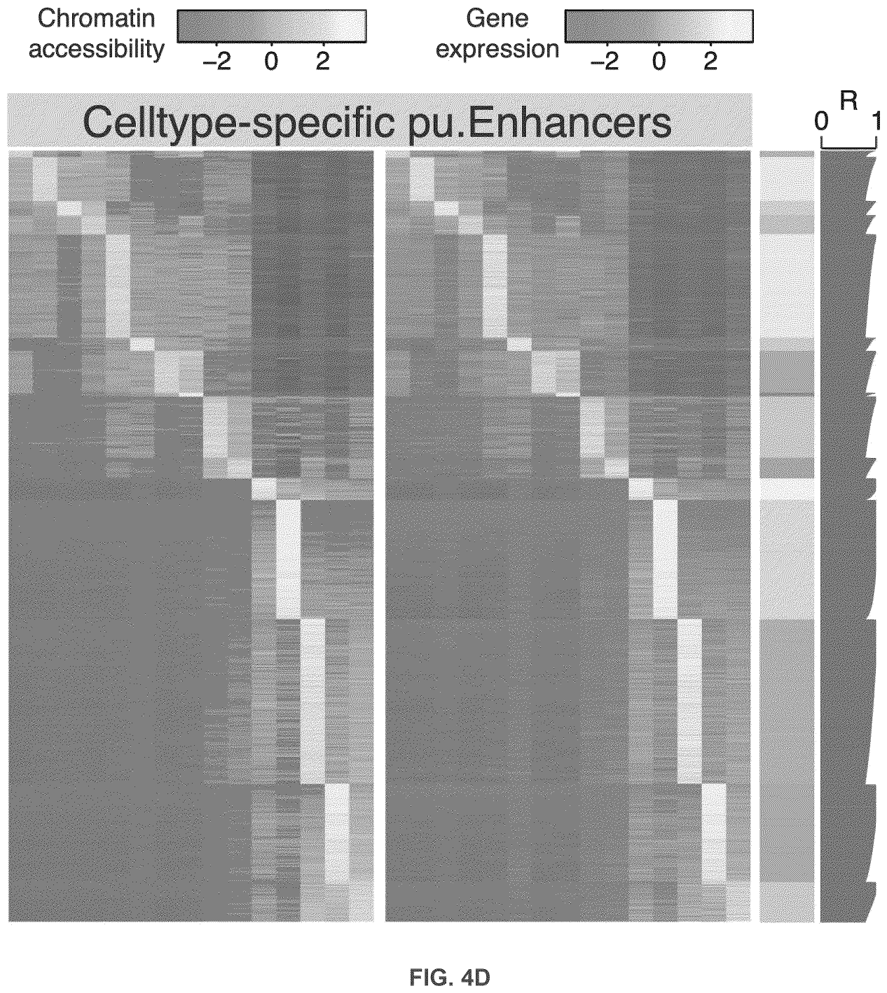

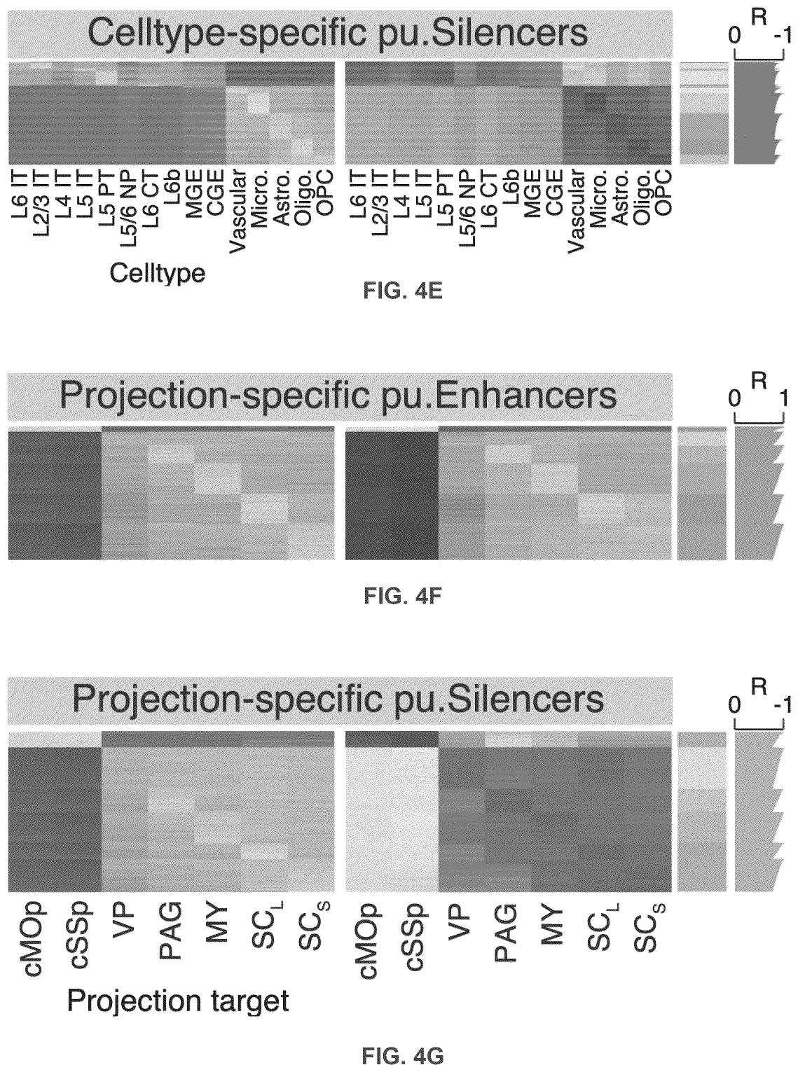

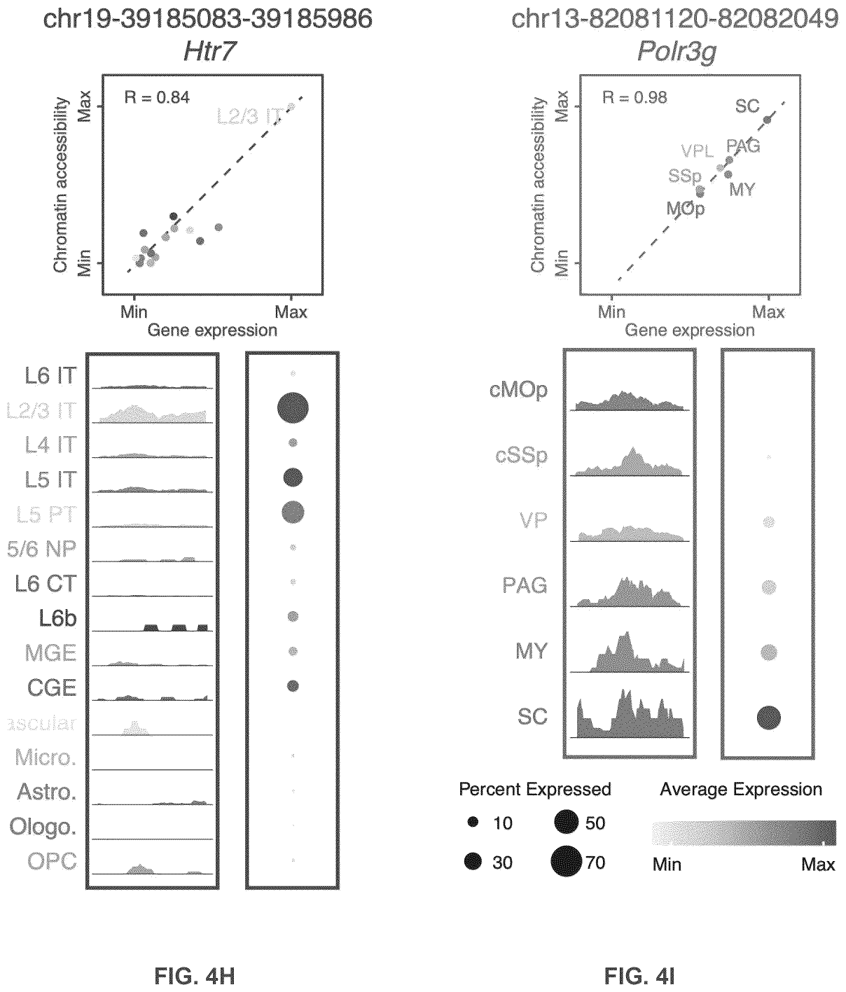

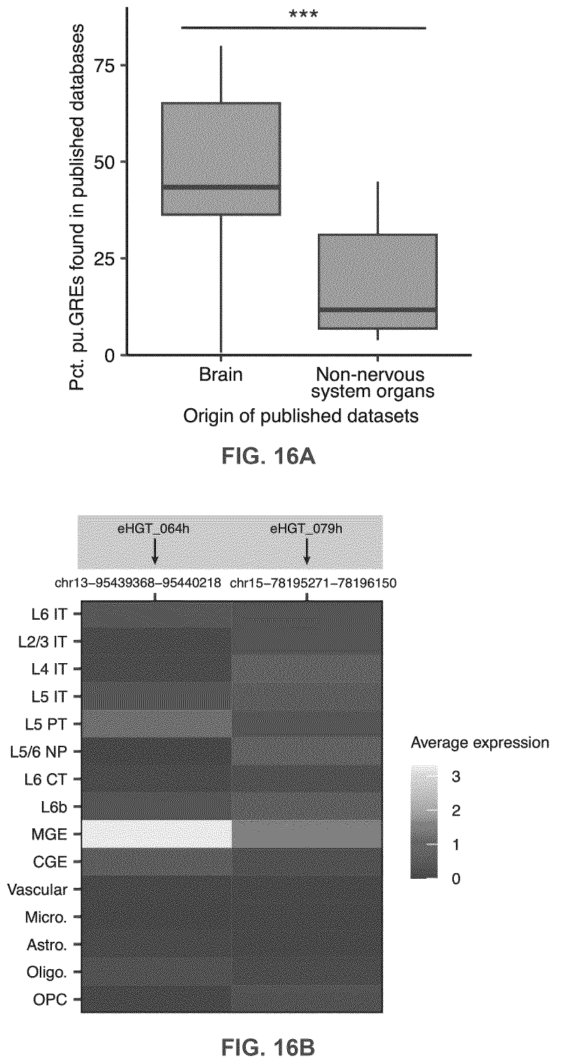

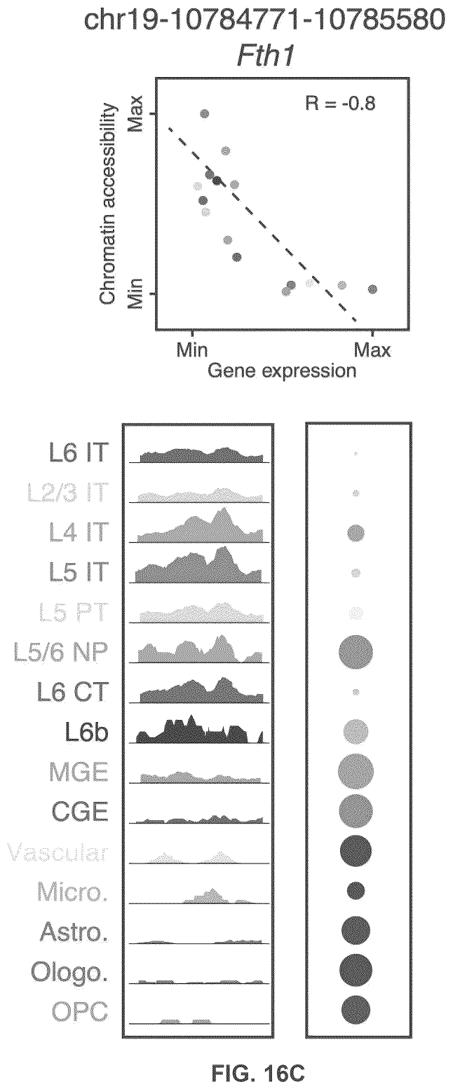

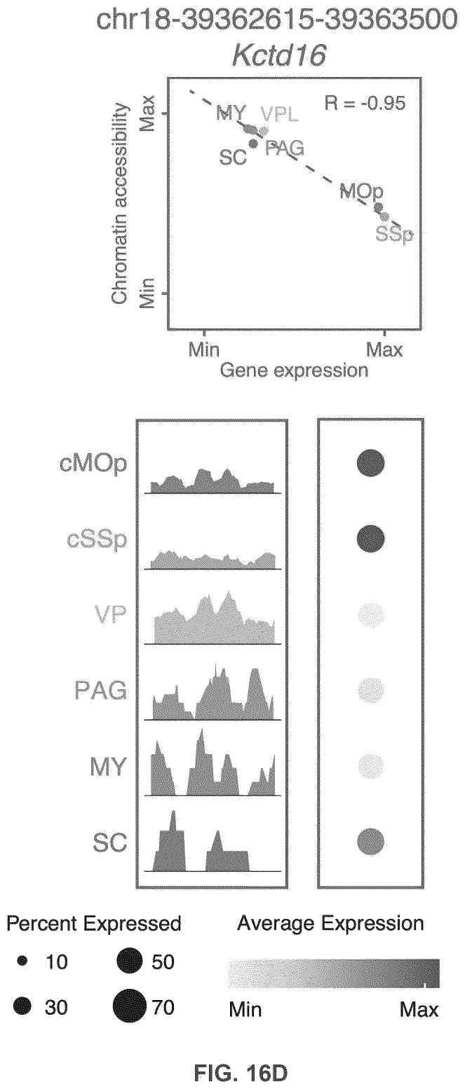

To this end, the snRNA-seq and snATAC-seq data from MOp and SSp was integrated to produce a unified, multi-modal cell census. 166,540 peaks were identified when aggregated across all snATAC-seq libraries. Many of those peaks likely contain functionally relevant GREs, as 85.6% of the peaks are located at the distal regions in the genome (FIG. 4A), and 43.9% of them are differentially accessible in individual transcriptional cell types and/or neurons projecting to different targets (FIG. 4B). To identify putative GREs and their correspondence to genes they regulate, 67,541 D peaks (Log 2FC >1, FDR <0.05) were identified in transcriptional cell types and 30,843 DA peaks in neurons projecting to each target, the average expression of genes from snRNA-seq data was then calculated and the average accessibility of DA peaks from snATAC-seq data in each cell types or projections. Pearson's correlation was calculated between the accessibility of DA peaks with the expression of genes for any peak-gene pairs that are within a 5M bp window of the same chromosome (FIG. 4C). A peak-gene pair with a strong positive correlation (Pearson's r >0.75) is identified as a putative enhancer (pu.Enhancer) and its putative regulated gene, whereas a pair with strong negative correlation (Pearson's r<−0.75) is categorized as a putative silencer (pu.Silencer) and its regulated gene. 18,088 pu.Enhancer-gene pairs and 2,739 pu.Silencer-gene pairs were identified that are cell type-specific (FIG. 4D, FIG. 4E), and 3,545 pu.Enhancer-gene pairs and 4,200 pu.Silencer-gene pairs that are projection-specific (FIG. 4F, FIG. 4G). Several pieces of evidence support the authenticity of identified putative GREs. First, they exhibited a greater overlap with the DNase hypersensitive sites (96.4%) detected in the adult mouse brain compared to randomly selected genomic regions (42.7%) of similar sizes and GC contents⁸². Second, these putative GREs show significant alignment with GREs previously annotated by the ENCODE consortium, particularly those identified in the mouse brain as opposed to other organs (FIG. 16A). Furthermore, analysis reveals four experimentally validated functional enhancers that coincide with peaks identified in snATAC-seq data. Notably, two of these validated enhancers overlap with the cell type-specific pu.Enhancers identified in the analysis, and the predicted transcriptional cell types aligned with the cell types demonstrating the highest enhancer activity as verified experimentally (FIG. 16B).

Among identified putative GREs, the peak 39,185,083-39,185,986 on chromosome 19 is ˜3.2 Mbp upstream of the TSS of Htr7 gene (FIG. 4H). This peak, differentially accessible in L2/3 IT neurons, is positively correlated (Pearson's r=0.84) to Htr7, which is differentially expressed in the same cell type, suggesting that this peak may contain a pu.Enhancer that might drive the expression of Htr7 specifically in L2/3 IT neurons. The accessibility of peak 82,081,120-82,082,049 on chromosome 13 is positively correlated to the expression of Polr3g in neurons of different projections (FIG. 4I), Both the peak and gene are highly accessed/expressed in corticospinal neurons, suggesting this peak may contain a pu.Enhancer that might drive the expression of Polr3g specifically in the corticospinal projection neurons. Additionally, peaks chr19-10,784,771-10,785,580 and chr18-39,362,615-39,363,500 may contain cell type-specific and projection-specific pu.Silencers, respectively. While accessible in the genome, they may reduce the expression of Fth1 in the IT neurons and Kctd16 in ET-projecting neurons, respectively (FIG. 16C, FIG. 16D). Thus, Projection-TAGs offer a powerful, high-throughput platform to perform systemic multiomic analyses to gain insight into the gene expression and chromatin accessibility profiles of diverse projection neurons.

### Projection-TAGs Enable the Detection of Projection Neurons Tuned to a Behavioral Stimulus

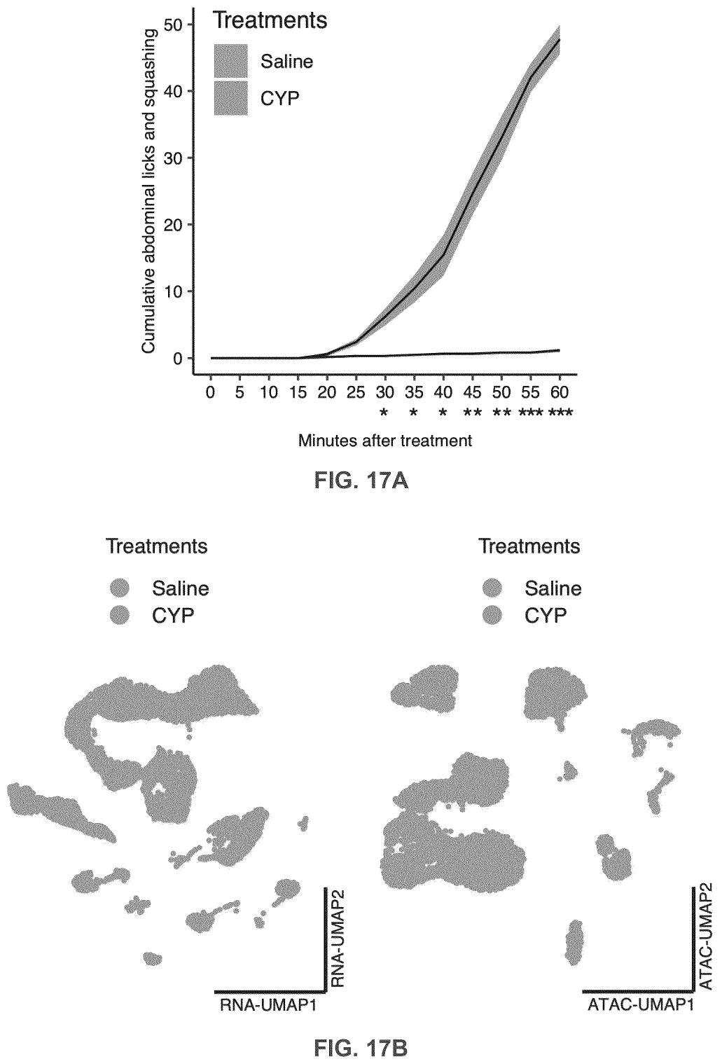

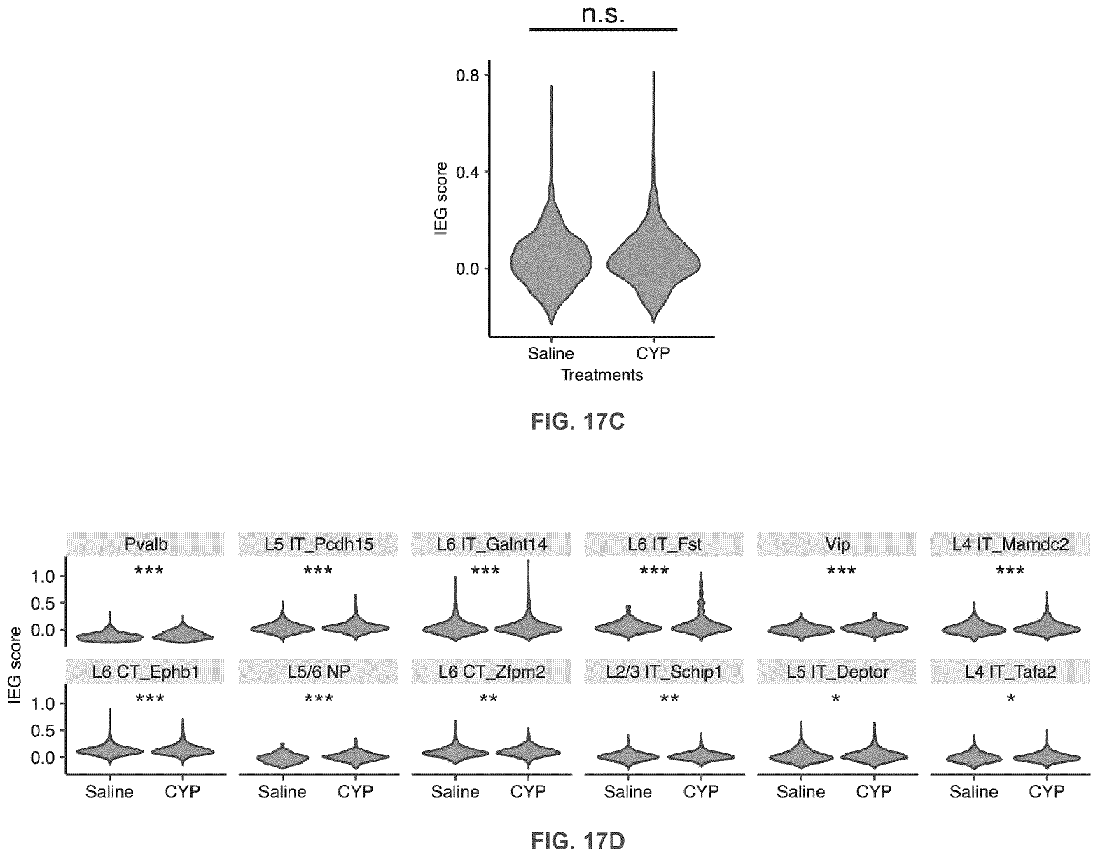

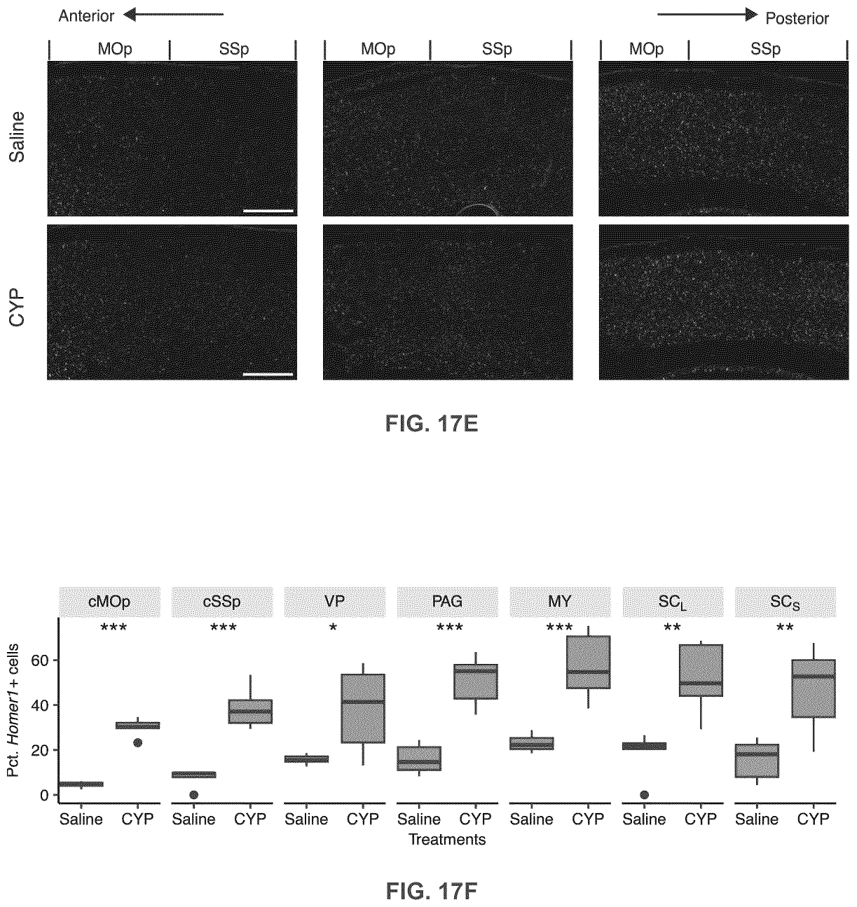

Projection-TAGs allow elucidation of spatial profiles, gene expression profiles, and chromatin accessibility profiles of diverse projection neurons at single-cell resolution. As Projection-TAG AAVs mediate stable BC expression with minimal immune response, this was leveraged to obtain a snapshot of activity-dependent recruitment of distinct projection neurons and their molecular features in the context of a specific stimulus. The MOp and the SSp circuitry have been extensively implicated in the modulation of distinct pain modalities, thus insight was needed into the gene expression changes in the cell types and their projections neurons in response to visceral pain stimulus. To study the cell populations acutely activated by visceral pain, the seven projection targets of the MOp and SSp were labeled as described herein elsewhere (FIG. 1A). On the day of experiment, acute inflammatory visceral pain was induced with cyclophosphamide (CYP) or inject saline as the control, followed by combinatorial snRNA-seq and snATAC-seq of the MOp and SSp 30 minutes after the stimulus, at which significant spontaneous behaviors were observed (FIG. 17A).

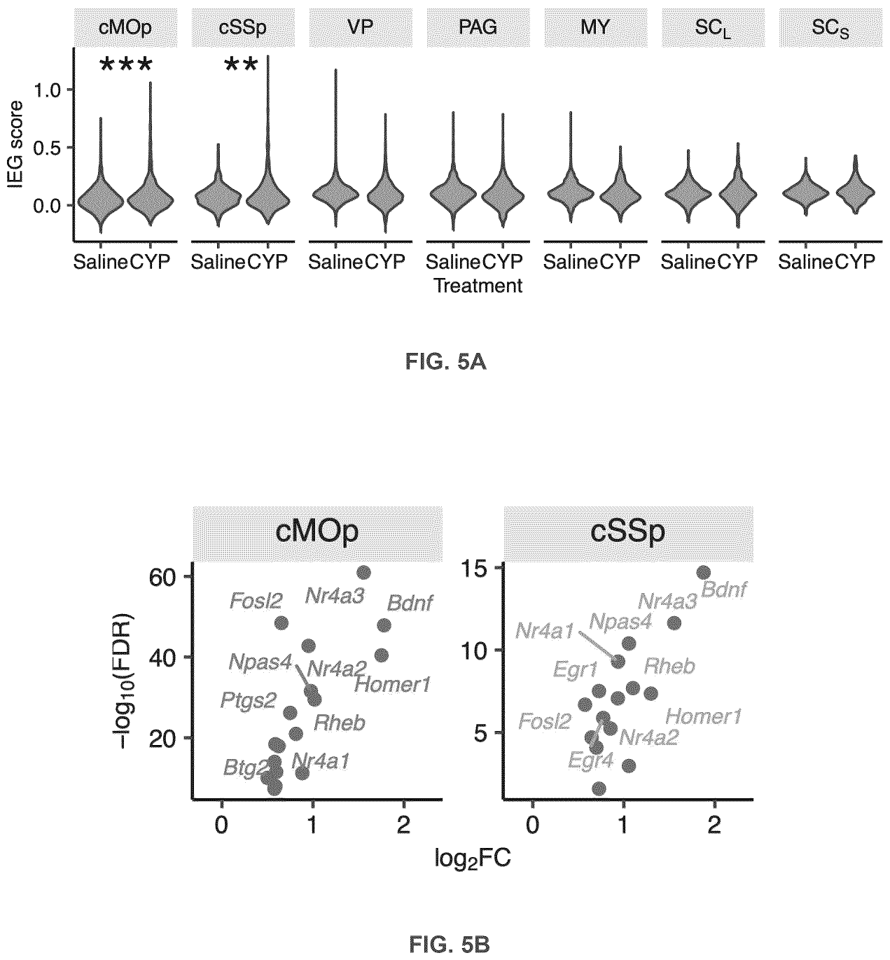

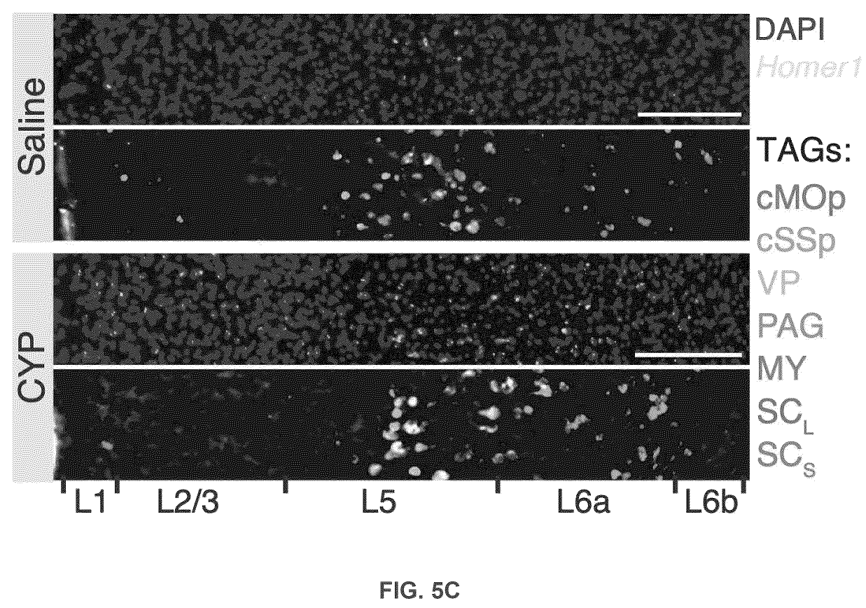

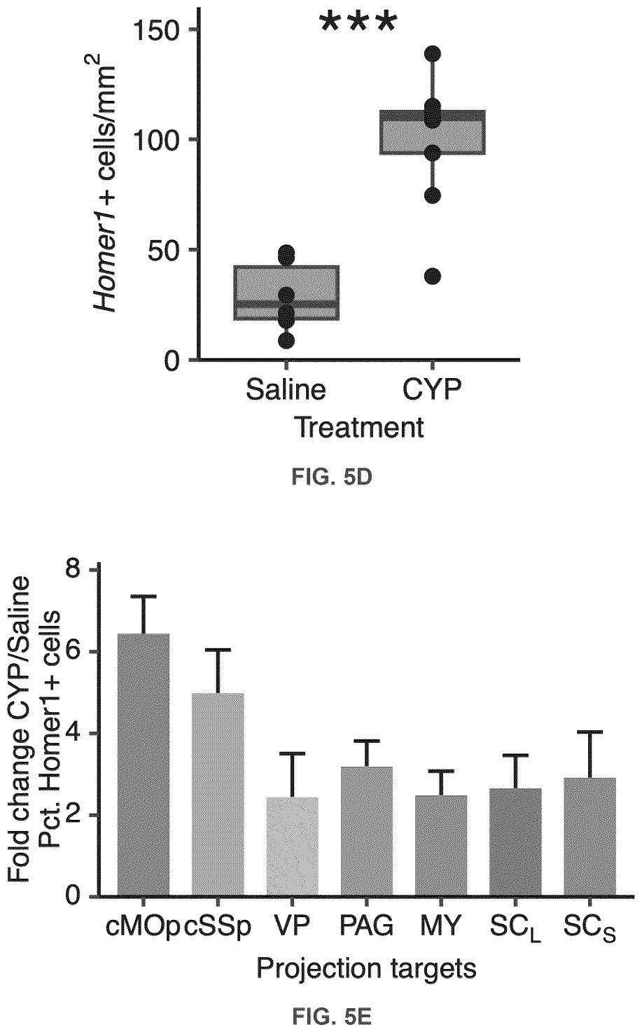

In both snRNA-seq and snATAC-seq data, nuclei from CYP- and saline-treated mice co-cluster together, suggesting visceral pain did not significantly alter the gene expression or chromatin accessibility of MOp and SSp cell types at the acute time point (FIG. 17B). To pinpoint the cell populations that are acutely activated by visceral pain, Act-seq was next applied, an approach that links the transcriptional activity following stimulus to individual neuronal cell types and projections based on the expression of immediate early genes (IEG). To aggregate expression across IEGs, an IEG score was generated for each snRNA-seq nucleus. While the IEG scores did not significantly differ in all nuclei between treatments (FIG. 17C), CYP significantly activated 12 transcriptional subtypes, including seven IT subtypes and two GABAergic subtypes (FIG. 12D), suggesting their preferential recruitments following the acute visceral pain stimulus. Further analysis of the projections suggests that CYP significantly activated only IT projections (both cMOp and cSSp), while none of the ET-projecting populations exhibited significant activation (FIG. 5A). Results pinpointed the cell types and projections that visceral pain selectively recruits in the acute phase of the pain induction state.

Next, analysis was expanded to determine the IEG profiles of the activated IT-projecting neurons, as it has been recently appreciated that distinct stimuli exhibit varying IEG activation profiles. DE analysis comparing all nuclei of the same projection between treatments failed to identify any CYP-induced IEGs. To maximize “signal-to-noise” ratio, transcriptionally “activated” nuclei were identified based on their IEG scores, followed by DE analysis comparing activated nuclei from CYP-treated mice to inactivated nuclei from Saline-treated mice. It was found that CYP induced 17 IEGs in activated neurons projecting to IT targets (Log 2FC >0.5 in either cMOp or cSSp, FIG. 5B). Among them, Nr4a3, Bdnf, Rheb, and Homer1 showed higher fold change (Log 2FC >1) in both cMOp- and cSSp-projecting neurons. Interestingly, CYP did not significantly induce Fos expression (FIG. 5B, FIG. 17E). Homer1 expression is directly linked to neuronal activity and synaptic plasticity. To validate these findings, multiplexed FISH screening was performed for Homer1 as an activity-dependent marker for the CYP-induced pain state. It was observed that CYP increased Homer1 expression in MOp and SSp compared to saline (FIG. 5C, FIG. 5D). Next, to investigate if Homer1 is selectively enriched in the IT-projecting neurons following visceral pain, the colocalization of Homer1 with each Projection-TAG was examined. While CYP induced Homer1 expression in all projection pathways analyzed (FIG. 17F), IT-projection neurons exhibited a higher magnitude of Homer1 induction, as the percentage of Homer1+ cells increased by 6.4 and 5-fold in cMOp and cSSp-projecting neurons, compared to on average 2.7-fold in the ET-projecting neurons (FIG. 5E). In summary, data shows that Projection-TAGs could be readily implemented with transcriptional analysis such Act-seq to identify the molecular markers and recruitment of distinct specific brain-wide projections and cell types following the stimulus of interest.

### Discussion

The present disclosure describes development of Projection-TAGs, a retrograde AAV platform for multiplex neuroanatomical studies and high-throughput multi-modal profiling of projection neurons. Projection-TAG AAVs retrogradely label distinct projections with unique RNA barcodes with high specificity and efficiency. It can be easily adaptable to existing neuroscience workflow and is optimized for commercial assays, such as multiplexed FISH, which allows simultaneous spatial integration of multiple projections in the same animals, and single-cell profiling assays that enable multiomic profiling of projection neurons. Herein, Projection-TAGs were applied to examine the transcriptional and epigenetic landscapes of the cortex using combinatorial snRNA-seq and snATAC-seq. Projection-TAGs could facilitate the development of novel tools for projection-specific targeting/manipulation by opening new avenues for studying multiple projections in the same animals and for identifying key gene expression features and genomic regulatory elements in distinct cortical cell types and diverse projection neurons. Lastly, it was demonstrated that Projection-TAGs can be incorporated with additional experimental paradigms, providing users with flexibility for studying activity-dependent recruitment of distinct cell populations and projections in the stimulus of interest.

### Available High-Throughput Neuroanatomy Tools

RNA barcode-based high-throughput neuroanatomical tools have greatly expanded the multiplexing capacity of neuronal tracing. Available high-throughput neuroanatomical techniques can be broadly classified based on their retrograde and barcoding schemes. Anterograde tracing techniques, namely MAP-seq and its derivatives, such as BRICseq, BARseq, and BARseq2. MAPseq utilizes a sindbis viral library to encode a diverse collection of short random RNA barcodes, which act as the cell barcode/identifier. The sindbis viral library is injected into the source region, and a unique barcode is expressed in individual neurons and anterogradely transported to the axonal terminals. Multiplexed projection tracing is achieved by assaying the RNA barcodes present in each of the target regions. MAPseq can be coupled with scRNA-seq for multi-modal profiling of projection neurons and with in-situ sequencing for spatial analysis (BARseq) and spatially-resolved transcriptional assays (BARseq2). Due to the anterograde tracing scheme, BARseq and BARseq2 can achieve high spatial resolution in both source and target regions, but the multiplexing capacity and tracing accuracy of MAPseq may be limited by tissue dissections. With the simple surgery procedure (one injection into the source region), MAPseq and derivatives are ideal for quantitatively measuring the projection strength. However, the requirement of highly customized instruments and pipelines by those methods has restricted their adoption beyond several expert labs. The high replication rate of sindbis virus results in cellular toxicity, posing a challenge to integrating their use with chronic experimental paradigms.

Retrograde tracing techniques, including Projection-seq, MERGE-seq, and Projection-TAGs. These techniques utilize a limited number of RNA barcodes with known sequences, which act as the projection barcode/identifier. A retrograde AAV expressing a unique BC is injected into each of the target regions, which retrogradely label projection neurons in the source region. Multiplexed projection tracing is achieved by assaying the barcodes present in the source region, which can be simply read out by scRNA-seq and spatial assays. Due to the retrograde tracing scheme, Projection-TAGs and similar techniques can achieve high spatial resolution in the source region, but not necessarily the target regions. The surgery procedures are relatively complicated (one injection into each of the target regions), which may limit the multiplexing capacity, and the accuracy of targeting each target region may introduce technical variations that confound the quantitative measurement of the projection strength. Surgical targeting (one injection into each of the target regions) may introduce technical variations that confound the quantitative measurement of the projection strength and might limit the multiplexing capacity. Despite these technical challenges, barcode detection is relatively simple and flexible and can be achieved using various commercial assays. Projection-TAGs enable multiplex neuroanatomical studies and high-throughput multi-modal profiling of projection neurons. AAVs have minimal cellular toxicity, which is ideal for incorporating Projection-TAGs with additional experimental paradigms for studying activity-dependent recruitment of distinct cell populations and projections in the stimulus of interest.

### Consideration for Projection-TAG Experimental Design and Result Interpretation

Projection-TAGs allow multiplex projection tracing and multi-modal profiling of projection neurons. In retrograde viral tracing experiments, the cargo gene expression in a single cell depends on a series of biological processes such as viral attachment and internalization, transduction and trafficking to the cell body, and escape and entry into the nucleus. rAAV2-retro has been widely adopted in neuroscience laboratories, Projection-TAG rAAV2-retro performance would be similar to other rAAV2-retro viruses, and prior experience using rAAV2-retro may be used to guide the planning of Projection-TAG multiplex tracing experiment. Efficient and accurate labeling of Projection-TAGs relies on successful targeting of desired brain regions, transduction efficacy of rAAV2-retro, titer (Projection-TAG AAVs with the titer of ˜2e+12 vg/ml label 14.7±8.4% of MOp cells, compared 3.7±3.0% by those with the titer of ˜5e+11 vg/ml) and volume of the viruses injected. The high correlation between Projection-TAG (RNA level) and GFP (protein level) expressed using the Projection-TAG AAVs provides a simple strategy for investigators to estimate retrograde tracing efficiency and accuracy of target labeling, which are essential for a successful Projection-TAG experiment. While Projection-TAG plasmids have the potential to be flexibly packaged into other AAV serotypes, the transduction efficiency and tissue/cell type tropisms related to AAV serotypes may lead to false negatives and should be considered while designing the experiments and interpreting the results. While Projection-TAG AAVs can be multiplexed and used interchangeably (FIG. 8D, FIG. 8E, FIG. 8F, FIG. 8G, FIG. 8H, FIG. 9H), AAV transduction efficiency and Projection-TAG expression may not be uniform across projection pathways (FIG. 9F, FIG. 9G), which may introduce confounds to quantitative measurement of the projection strength.

After the tracing experiments, Projection-TAG detection and multi-modal profiling can be achieved using various commercial assays. Projection-TAG detection is specific and sensitive in single-cell sequencing and multiplexed FISH, as described (FIG. 1A). Multiplexed FISH is superior at the Projection-TAG detection efficiency but is labor-intensive and difficult to scale up. Sun1-GFP expressed by Projection-TAG AAVs reliably labels both whole cells and nuclear suspensions, enabling enrichment of projection neurons for unbiased single-cell and single-nucleus transcriptional and epigenetic profiling in a high-throughput manner. Enzyme-free nuclear extraction works for various tissue samples (fresh, frozen, or fixed) and introduces minimal dissociation-induced transcriptional stress response, and thus ideal for activity-dependent circuit mapping. W hole-cell sequencing has the ability to sequence both cytoplasmic and nuclear transcripts, advantageous for recovering medium- to low-expressing transcripts and may increase Projection-TAG detection. Sequencing involves many biochemical processes that may confound the Projection-TAG detection. Artifacts such as ambient RNA contamination, doublets, and analytical errors may lead to false positives, which can be addressed experimentally (e.g. reduce ambient RNA contamination by adding additional wash steps and incorporating FACS) and computationally (e.g. ambient RNA correction, doublet removal, and setting up appropriate FDR for the analysis). Projection-TAG detection rate is limited by the sequencing assays (e.g. efficiency of reverse transcription and cDNA capture) and sequencing setups (FIG. 9A), which may pose an upper limit for Projection-TAG detection and lead to false negatives. Consequently, lack of Projection-TAG expression in a cell should not be simply interpreted as lack of projection, and the neurons projecting to each target may be under-reported in snRNA-seq, which may result in a higher degree of underestimation of the axonal collaterals. Projection-TAG detection using sequencing assays with higher transcript capture efficiency and multiplexed FISH (or multi-color CTB tracing) may circumvent false negatives.

### Applications of Projection-TAGs

Projection-TAGs can be compatible with additional commercial platforms, such as high-throughput spatial transcriptomics platforms like Xenium and Visium, that would allow spatially-resolved investigation of diverse cell types and distinct anatomical organization of the projection neurons. Another application of Projection-TAGs includes connecting neuronal activity to the distinct projections by integrating the oScarlet version with in vitro and in vivo calcium imaging experiments. The cell type and projection information from imaging-based molecular assays can be integrated with the real-time neuronal activity information from the calcium imaging experiments via post-hoc imaging alignment and registration.

The number of projections that can be labeled with Projection-TAG AAVs is not inherently constrained. Projection-TAGs can be easily scalable, including 50 screened BCs described herein that may be packaged to increase multiplexing of projection tagging. It has been well appreciated that AAV serotypes have distinct tropism and selective labeling of the brain nuclei. With the discovery of capsid selection using directed evolution and advances in sequencing techniques and computational tools to optimize exogenous transcript detection, that the improved retrograde labeling efficiency of viruses is anticipated to enhance wider applications of Projection-TAGs. Given its flexibility, usability, and compatibility with commercial platforms, Projection-TAGs can be readily applied to study diverse projection types in the central and peripheral nervous system.

### Methods

### Generation of Projection-TAGs

100-bp BCs were used because they allow design HCR probes to detect the spatial distribution of BC transcripts, and would improve their detection rate in RNA-seq compared to shorter BCs that are commonly used. 60 previously reported BCs were retrieved, followed by filtering out the BCs that contain the sequence of restriction enzymes (BamHI, HpaI, and NotI). Next, sequence alignment was performed using blastn suite searching in the “Nucleotide Collection (nr/nt)” database and “refseq_representative_genomes” database against the genomes of human (taxid:9606), mouse (taxid:10090), rat (taxid:10116), and Primates (taxid:9443) and filtered out the ones that showed significant similarities. The Euclidean distance was calculated between any of the two BCs using DistanceMatrix( ) function in R package DECIPHER. The first 50 BCs sorted were cataloged and reported based on the average Euclidean distances with other BCs.

### Cloning and Viral Packaging of Projection-TAG AAVs

To generate the backbone of pAAV-CAG-Sun1-GFP-WPRE-pA, two previously reported plasmids were linearized: pAAV-CAG-H2B-GFP (Addgene, Plasmid #116869) using HindIII and pAAV-Ef1a-DIO-Sun1GFP-WPRE-pA (Addgene, Plasmid #160141) using AscI, and end filled, followed by digestion with SpeI and NheI, respectively, and gel extraction of the fragments at around 4,700 bp (pAAV-CAG backbone) and 2,400 bp (Sun1-GFP), respectively. Next the plasmid pAAV-CAG-Sun1-GFP-WPRE-pA was generated by ligating the two fragments together. To insert BCs into pAAV-CAG-Sun1-GFP-WPRE-pA, a gene block fragment was generated for each BC with the following structure (5′ overhang-NotI-BC-SV40 polyA-HpaI-3′ overhang) and sequence (AAGGAAAAAAgcggccgc (SEQ ID NO:51)-100 bp sequence of BC-ttcgagcagacatgataagatacattgatgagtttggacaaaccacaactagaatgcagtgaaaaaaatgctttatttgtgaaat ttgtgatgctattgctttatttgtaaccattataagctgcaataaacaagttaacAACCGCTGCCG (SEQ ID NO:52)). both pAAV-CAG-Sun1-GFP-WPRE-pA and BC gene blocks were then digested with NotI and HpaI, and generated pAAV-CAG-Sun1-GFP-WPRE-BC-pA by ligating the fragments. 12 plasmids were generated each expressing a unique BC (1-12). The plasmids were packaged into AAV (BrainVTA, China) and generated a set of 12 rAAV2-retro samples each expressing a unique BC (titer range 1.1-3.5e+12 vg/ml). To generate a set of 12 plasmids pAAV-CAG-oScarlet-WPRE-BC-pA each expressing a unique BC, the BamHI-oScarlet-WPRE-NotI gene fragment was generated and replaced the Sun1-GFP-WPRE fragment in pAAV-CAG-Sun1-GFP-WPRE-BC-pA using BamHI and NotI. All restriction enzymes were purchased from New England Biolabs, and all plasmids produced herein were deposited to Addgene.

### Testing Projection-TAGs in HEK Cells

Human embryo kidney cell line HEK 293T/17 (ATCC) was acquired and cultured using DMEM (Corning), supplemented with 10% Fetal Bovine Serum (Gibco), and 1% Penicillin/Streptomycin (Sigma), on either 60 mm dishes for RNA-seq or 8-well chambered slides (Nunc™ Lab-Tek™) pre-coated with PDL-collagen for multiplexed FISH. Once the cells reached 70-80% confluency, the DNA of one BC plasmid was transfected into each of the HEK samples using lipofectamine 2000 (Invitrogen) according to the manual. Cells were incubated for 24-36 hours before analysis. For RNA-seq, around 1 million cells were used for each sample and RNA was extracted and purified using RNAqueous™ Total RNA Isolation Kit (Invitrogen) following the manual. Library preparation and sequencing were conducted by the McDonnel Institute of Genomics at Washington University School of Medicine. For FISH, cells were washed with DPBS and fixed using 4% formaldehyde for 10 minutes at room temperature. FISH was performed following the manufacturer's manual.

### Animals

All experiments were conducted in accordance with the National Institute of Health guidelines and with approval from the Animal Care and Use Committee of Washington University School of Medicine. Mice were housed on a 12-hour light-dark cycle (6:00 am to 6:00 μm) and were allowed free access to food and water. All animals were bred onto C57BL/6J background, and no more than five animals were housed per cage. Female littermates between 8 and 10 weeks old were used for experiments.

### Stereotaxic Surgeries

Mice were given a single subcutaneous injection of 0.5 ml 0.9% sterile sodium chloride and Buprenorphine-SR, 1 hour prior to surgery to help rehydrate the mouse after anesthesia. Mice were anesthetized with 1.5-2% isoflurane in an induction chamber using isoflurane/breathing air mix. Once deeply anesthetized, mice were secured in a stereotactic frame (RWD Life Science) where surgical anesthesia was maintained using 2% isoflurane. Mice were kepton a heating pad for the duration of the procedure. Preoperative care included application of sterile eye ointment for lubrication, administration of 1 mL of subcutaneous saline, and surgery-site sterilization with iodine solution. Injection was performed into the seven projection targets of the cortex, each with a Projection-TAG (rAAV2-retro expressing a unique TAG), in single animals. All injections were made using a Nanoject II auto injector (Drummond) with a glass microelectrode at a rate of 1 nl/s and the needle was held in place for 10 minutes prior to needle withdrawal. Needles are changed between each Projection-TAG injection to avoid contamination between Projection TAGs. Stereotaxic surgery injecting into SC_{L} and SC_{S} was performed at week 0. For SC_{L} injection, the injections were located 400 μm lateral to the center of the posterior artery (150-300 mm below the dura), and the virus was bilaterally injected between T12 and T13 intravertebrally with 250 μl volume each injection. For SC_{S} injection, the dorsal part of the L1 vertebra was gently excised using a high-speed micro-drill (RWD Life Science) to unveil the L6-S1 spinal cord segments. The injections were located 100 μm left-lateral to the center of the posterior artery with a 10-degree left-right tilt (550-580 mm below the dura). Three distinct sites were injected with 150 nl of virus at each site. Following a 2-week convalescent interval, the second stereotaxic surgery was conducted by injecting into cMOp, cSSp, VP, PAG, and MY using the following coordinates relative to Bregma: cMOp (anterior-posterior [AP] −0.52 mm, medial-lateral [ML]+1.81 mm, dorsal-ventral [DV] −0.86 mm, left hemisphere), cSSp (AP −0.52 mm, ML+0.75 mm, DV −0.86 mm, left hemisphere), VP (AP −1.5 mm, ML −1.52 mm, DV −4 mm, right hemisphere), PAG (AP −4.5 mm, ML −0.5 mm, DV −2.83 mm, right hemisphere), and MY (AP −6.2 mm, ML −0.5 mm, DV −5.93 mm, left hemisphere). A small midline dorsal incision was performed to expose the skull. After leveling the head relative to the stereotaxic frame, the specified injection coordinates were used to mark the locations on the skull, and a small hole (approximately 0.5 mm diameter) was drilled for each. 500 nl of virus was injected into each of the targets. After each surgery, the surgery site was sutured, and mice were recovered from anesthesia on a heating pad and then returned to their home cage. Mice were given carprofen (0.05 mg/ml in water) to minimize inflammation and discomfort and monitored for three consecutive days.

To label neurons co-projecting to cSSp and VP, 500 nl of AAV2retro-hSyn-eGFP-Cre (Addgene #105540) was injected into VP and 500 nl of AAV2retro-Ef1a-DIO-H2B-tdTomato (BrainVTA) into cSSp of the same mice (n=2) using the stereotaxic coordinates described above. Retrograde labeling in the SSp and MOp was examined two weeks after injection.

### Mouse Model of Visceral Pain

Stereotaxic surgeries were performed as described above. At week 5 post-viral injections, mice were acclimated for at least 3 days prior to the experiment. Mice were administered an intraperitoneal injection of either cyclophosphamide (Sigma-Aldrich, dissolved and diluted to 40 mg/mL in 0.9% sodium chloride) with a dose of 200 mg/kg or saline (approximately the same volume as CYP). Animals were monitored in their home cage for 30 minutes before perfusion.

### Tissue Processing

In the experiments reported in this paper, the seven projection targets of the MOp and SSp were traced by performing two stereotaxic surgeries using undiluted AAVs in 8-10 weeks-old C57BL/6 female mice. Individual Projection-TAG AAVs were injected into the SC_{L} and SC_{S} at week zero, followed by a second surgery of AAV injections into the MOp, SSp, VP, PAG, and MY at week two. Animals were perfused, cortical samples were collected for either FISH or sequencing analysis at week five.

To collect biological samples for FISH studies, animals were anesthetized with ketamine cocktail, and perfused with DEPC-PBS followed by 4% DEPC-PFA. Brain and spinal cord tissues were dissected and incubated in 4% DEPC-PFA at 4 C for 6-8 hours. Tissues were then incubated in 30% sucrose in 1×DEPC-PBS at 4 C for 24-48 hours until they sank to the bottom of the tube. Tissues were then embedded in OCT and stored at −80° C. before slicing. Tissues were sliced coronally into sections with 30 μm thickness using a cryostat (Leica), and tissue slices were mounted on microscope slides. Slides were stored at −20° C. for a least one hour before moving to −80° C. for long-term storage. Slides containing the brain regions of injections were examined under a fluorescent microscope, and the fluorescent signal from Sun1-GFP was used to confirm the injection sites. As an optional step, the native fluorescence of Sun1-GFP can be photobleached from mouse brain sections by floating slices in 1×PBS with 24 mM NaOH and 4.5% H₂O₂ and exposing under the UV light (27 total watts, OPPSK) for 30 minutes at room temperature. Slices were immediately rinsed with DEPC-PBS twice before mounting on a microscope slide.

To collect biological samples for molecular experiments such as qPCR or sequencing, animals were anesthetized with ketamine cocktail, and perfused with ice-cold NMDG-based cutting solution (NMDG 93 mM, KCl 2.5 mM, NaH₂PO₄ 1.25 mM, NaHCO₃30 mM, HEPES 20 mM, Glucose 25 mM, Ascorbic acid 5 mM, Tiourea 2 mM, Sodium Pyruvate 3 mM, MgSO₄ 10 mM, CaCl₂ 0.5 mM, N-acetylcysteine 12 mM; pH adjusted to 7.3 with 12N HCl, and bubbled with 95% 02 and 5% CO₂). The spinal cord was dissected, fixed, and sectioned by following the sample preparation process mentioned above in order to confirm the injection sites of SC_{L} and SC_{S}. The brain was submerged in ice-cold NMDG-based cutting solution and sliced coronally into sections with 400 μm thickness using a Compresstome (Precisionary, VF-210-0Z). MOp and SSp samples were prepared by micro-dissecting the respective regions under a microscope and collected into a nucleus-free centrifuge tube placed on dry ice. Samples were stored at −80° C. for long-term storage. To assess the viral spread in the injection sites, the remaining brain and spinal cord sections containing the injection sites were placed on glass slides. The fluorescent signal from Sun1-GFP was examined under a fluorescent microscope assess viral spread and off-target labeling in the injection sites. To assess off-target labelling, it was examined if there were strong GFP signals displayed in the neighbor regions of the injection site, and if there is leaked labelling in the injection track it was considered as missed or off-target labelling.

Multiplexed FISH with HCR

HCR (hybridization chain reaction) v3.0 probes, amplifiers, and reagents were purchased from Molecular Instruments. Multiplexed FISH was performed according to the manual of HCR RNA-FISH with minor modifications. For hybridization, samples were equilibrated in hybridization buffer for 30 min at 37° C. and hybridized with probe sets (16 nM each probe) in hybridization buffer overnight at 37° C. Samples were washed in probe wash buffer and gradually switched to 5×SSCT (5×SSC, 0.1% Tween-20) at 37° C. HCR was carried out at room temperature. Samples were equilibrated in amplification buffer for 30 min and the amplifier hairpins (conjugated to Alexa-488, Alexa-546, Alexa-647, and/or Cyanine 7) were heated to 95° C. and snap-cooled in a dark drawer for 30 min. Hairpins were then mixed and diluted to 0.6 nM each hairpin in amplification buffer before incubated with samples overnight. Samples were washed with 5×SSCT for a total of three times and imaged in 2×SSCT with DAPI. Imaging was carried out as described in the next section. After imaging, samples were washed with 5×SSCT, then HCR probes and hairpins were stripped by incubating with 0.25 U/ul of DNase I (Sigma Aldrich) in 1×DNase incubation buffer for 90 minutes at 37° C. (FIG. 7G, FIG. 7I). Samples were washed with 5×SSCT for 5 minutes for a total of 5 times before the next round of HCR-FISH.

### Imaging

After each FISH round, slices were imaged at 10× magnification on a fluorescence microscope (Keyence, BZ-X800). Filter cubes used include DAPI (Chroma 49021, Excitation filter at 405 nm with 20 nm bandwidth, Emission filter at 460 nm with 50 nm bandwidth), GFP/AF488 (Chroma 49011, 480/40x, 535/50m), Alexa-546 (Chroma 49304, 546/10x, 572/23m), Alexa-647 (Chroma 49006, 620/60x, 700/75m), and Cyanine 7 (Chroma 49007, 710/75x, 810/80m). The DAPI signal and signals from FISH probes were imaged for each FISH round. Images from each FISH round were stitched using the Keyence BZ-X800 Analyzer. Images from multiple FISH rounds were loaded into FIJI/ImageJ and aligned using the HyperStackReg plugin by choosing DAPI channels for transformation matrix computation. Images were then downsampled by a factor of 2 and background was subtracted.

### Quantification of Projection Neurons Using QUINT

FISH images were segmented and quantified using the QUINT workflow which allows for semi-automated quantification of cells in labeled brain regions. For atlas registration, images from different channels were merged and downsampled according to the manual. An XML file was generated using Filebuilder and loaded into QuickNII for linear registration to the Allen Mouse Brain Atlas CCFv3. After, user-guided nonlinear refinements were applied to brain sections using VisuAlign. For the segmentation of positive cells, images of individual channels were prepared, and segmentation was performed through the pixel classification and object classification pipelines in ilastik. In pixel classification, models were trained to distinguish signal from background using a small subset of images and applied to the whole dataset. Individual machine-learning algorithms were trained for each channel. Probability maps of signal and background were exported as HDF5 files to be used in object classification. In object classification, models were trained to distinguish objects from nonspecific labeling based on features such as size and shape using a small subset of images applied to the whole dataset.

The performance of illastik segmentation algorithms was validated against manual segmentation. Illastik accurately identified 96.8% of manually segmented cells with a 6.6% false positive rate (FIG. 11B). There are 2.1% of the segmented objects identified as multiple cells merged into one object and 1.9% of the segmented objects identified as one cell split into multiple objects. Finally, segmentation and registration files were uploaded to the Quantifier feature in Nutil which resulted in quantification of cells per brain region according to the reference atlas. To identify cells that co-labeled by multiple markers, objects positive for each marker were segmented individually and the x and y coordinates of the center of each object were calculated using illastik. If the Euclidian distance of two objects from separate channels is less than the average radius of all objects, the two objects were identified as one cell that co-expresses both marker genes. For Projection-TAG detection and specificity analysis, cSSp and cMOp were excluded from this analysis because they are known to contain neurons projecting to other injection sites and may be labeled by TAGs injected into other regions which could contaminate the final distribution of the TAG.

### Single-Nuclei Isolation and FACS

Nuclear extraction was performed according to the protocol described previously with minor modifications. Mouse cortical tissues were transferred to a dounce homogenizer in homogenization buffer (0.25 M sucrose, 25 mM KCl, 5 mM MgCl2, 10 mM Tris-HCl, pH 8.0, 5 μg/mL actinomycin, 1% BSA, and 0.08 U/ul RNase inhibitor, 0.01% NP40) on ice. Samples were homogenized for 10 strokes with the loose pestle in a total volume of 1 mL, followed by 10 additional strokes with the tight pestle. The tissue homogenate was then passed through a 50 μm filter and diluted 1:1 with working solution (50% iodixanol, 25 mM KCl, 5 mM MgCl2, and 10 mM Tris-HCl, pH 8.0). Nuclei were layered onto an iodixanol gradient after homogenization and ultracentrifuged as described previously. After ultracentrifugation, nuclei were collected between the 30 and 40% iodixanol layers and diluted with resuspension buffer (1×PBS with 1% BSA, and 0.08 U/ul RNase inhibitor). Nuclei were centrifuged at 500 g for 10 min at 4° C. and resuspended in resuspension buffer with 5 ng/ul of 7-AAD. For FACS, gates on GFP and 7AAD were set using tissues collected from mice with no stereotaxic surgeries and no 7AAD staining. GFP+/7-AAD+ events and GFP−/7-AAD+ events were sorted using 100 μm nozzle on a BD FACSARIA II and collected separately into 1.5 mL microcentrifuge tubes containing 100 μl of resuspension buffer.

snRNA-Seq and snATAC-Seq

For snRNA-seq, nuclei were further processed and sequenced according to the manufacturer's manuals of Parse-biosciences Evercode WT mini Kit V2 (Parse) or 10× Genomics Chromium Single Cell Gene Expression 3′ V3.1 Assay (10×RNA). For combinatorial snRNA-seq and snATAC-seq, nuclei were processed and sequenced according to the manufacturer's manual of 10× Genomics Chromium Single Cell Multiome Assay (10× Multiome). Libraries were sequenced on a NovaSeq6000 with 150 cycles each for Read1 and Read2, targeting 100,000 paired reads/nucleus for snRNA-seq libraries and 50,000 paired reads/nucleus for snATAC-seq libraries. Raw sequencing data from individual libraries were processed and mapped using 10× Genomics cellranger-6.1.2 (10×RNA), 10× Genomics cellranger-arc-2.0.1 (10× Multiome), or Parse Computational Pipeline v1.1.2 (Parse). The reference genome was generated by adding the Sun1-GFP and Projection-TAGs 1-12) to GRCm38. Projection-TAG feature was extended from the 100-nt Projection-TAG sequence by 140-nt on each end, to ensure sequencing reads have at least 10-nt alignment to the Projection-TAGs. For snATAC-seq, accessible peaks were identified using cellranger-arc aggr by analyzing the combined fragment signals across all snATAC-seq libraries in the dataset.

### Target Amplification

Parallel PCR reaction (KAPA HiFi HotStart ReadyMix, Roche) was carried out to amplify Projection-TAG sequences from the cDNA libraries generated using 10× Genomics Chromium Single Cell Gene Expression 3′ V3.1 or Multiome Assay. First, forward primer (5′-ACACTCTTTCCCTACACGACGCTCTTCCGATCT-3′ (SEQ ID NO:53)) and reverse primer (5′-GTGACTGGAGTTCAGACGTGTGCTCTTCCGATCTCCTCCCCGCATCGATACCG-3′ (SEQ ID NO:54)) were used separately in PCR reactions with 20 ng of cDNA library each reaction. PCR program: (1) 95° C. for 3 min, (2) 98° for 20 s, 70° C. for 15 s, 72° C. for 20 s (10 cycles), (3) 72 for 1 min. The reactions containing forward and reverse primers were pooled and a second PCR reaction was done following the PCR program above. As a result, the region containing the Projection-TAG sequence, 10× cell barcodes, and UMI sequences will be preferentially amplified from the cDNA molecules. The target amplification amplicons were labeled with sample indices (Dual Index Plate TT SetA, 10× Genomics) and using P C R reaction with program (1) 95° C. for 3 min, (2) 980 for 20 s, 65° C. for 15 s, 72° C. for 20 s (12 cycles), (3) 72 for 1 min. PCR products were purified using SPRIselect beads sequenced on a NovaSeq6000 with 150 cycles each for Read1 and Read2, targeting 10M reads per library. Raw sequencing data from individual libraries were processed and mapped to individual Projection-TAGs using 10× Genomics cellranger-6.1.2.

Quality Control, Clustering, and Annotation of snRNA-Seq

The gene-cell count matrices from all snRNA-seq libraries were concatenated using R (V4.1.1) package Seurat (V4.4.0). To be included in the snRNA-seq analysis, nuclei were required to contain at least 500 unique genes, less than 15,000 total UMIs, and fewer than 5% of the counts deriving from mitochondrial genes. There were 69,657 nuclei that met these criteria. Raw counts were scaled to 10,000 transcripts per nucleus and log-transformed using NormalizeData( ) function to control the sequencing depth between nuclei. Counts were centered and scaled for each gene using ScaleData( ) function. Highly variable genes were identified using FindVariableFeatures( ) and the top 20 principal components were retrieved with RunPCA( ) using default parameters. For dimension reduction and visualization, Uniform Manifold Approximation and Projection (UMAP) coordinates were calculated using RunUMAP( ). Nuclei clustering was performed using FindClusters( ) based on the variable features from the top 20 principal components, with the resolution set at 0.6, and the marker genes for each cluster was identified using FindAllMarkers( ) comparing nuclei in one cluster to all other nuclei. Doublet or low-quality nuclei were identified if they meet any of the following criteria: 1). Assigned to a cluster with no significantly enriched marker genes (FDR <0.05, log 2FC >1); 2). Assigned to a cluster in which five or more mitochondrial genes were identified among top 20 marker genes (sorted by avg_log 2FC); 3). Identified as doublets using R package DoubletFinder with doublet expectation rate at 5%. In total 5,991 nuclei were identified as doublet or low-quality and thus excluded from the dataset. The remaining 63,666 nuclei were clustered as described above.

Transcriptional classes and cell types were assigned to each cluster based on the canonical marker genes previously reported (FIG. 2E). Specifically, for classes, glutamatergic and GABAergic neuronal clusters are annotated based on the expression of Slc17a7 and Slc32a1, respectively. Non-neuronal clusters are annotated based on the lack of expression of Slc17a7 and Slc32a1. For cell types, IT clusters are annotated based on the expression of Slc30a3, and PT, NP, CT clusters are labeled by Lratd2, Lypd1, and Syt6, respectively. L6b is marked by Nxph4. Vascular cells express Crh and/or Uaca. Microglial cells and astrocytes are marked by C1qa and Emx2, respectively. Oligodendrocytes and OPC are labeled based on the expression of Mbp and Sox10. Subtypes were assigned to each cluster based on the marker gene uniquely expressed in that specific cluster (FIG. 12E, FIG. 12F).

### Projection Feature Annotation

To study the projection feature of single neurons, the projection targets were assigned to individual snRNA-seq nuclei based on the expression of Projection-TAGs. In snRNA-seq, a Projection-TAG-cell matrix was generated from the gene-cell matrix. A projection target is assigned to a single nucleus if that nucleus expresses the Projection-TAG (>0 UMIs) injected into the target, and each nucleus is evaluated for each projection target based on the expression of the corresponding Projection-TAG. In target amplification, an expression cutoff was set for each Projection-TAG in each library. The cutoff is 1 UMI or X percentile of the corresponding Projection-TAG expression in the corresponding library (where the X equals the percentage of nuclei not expressing the corresponding Projection-TAG in the corresponding snRNA-seq library plus one), whichever comes greater. A projection target is assigned to a single nucleus if that the Projection-TAG expression is greater than the cutoff, and each nucleus is evaluated for each projection target based on the expression of the corresponding Projection-TAG. Finally, to combine results from snRNA-seq and target amplification, a projection target is assigned to a single nucleus if that target is assigned in either snRNA-seq or target amplification. Of note, a nucleus can be assigned multiple projection targets if it expressed multiple Projection-TAGs. In the analysis where individual projection targets are analyzed separately (e.g. FIG. 2D), nuclei were grouped based on whether they express the corresponding Projection-TAG. For example, snRNA-seq nuclei only expressing cMOp-TAG and co-expressing cMOp-TAG and other TAGs will be grouped for cMOp analysis. In the analysis where axonal collaterals were reported, nuclei were grouped based on the expression of each of the Projection-TAGs. For example, snRNA-seq nuclei only expressing cMOp-TAG and co-expressing cMOp-TAG and cSSp-TAG will be grouped separately for analysis. Projection groups with sample size no less than 60 nuclei (corresponding to 0.34% of the total snRNA-seq nuclei generated from FACS-sorted libraries, the upper limit of FDR) were reported in all analyses.

Anchoring Analysis of snRNA-Seq

To validate the transcriptional cluster annotation, snRNA-seq data was directly compared to a published scRNA-seq of mouse MOp and SSp previously described. Seurat was used to anchor snRNA-seq data to the published dataset. First, FindTransferAnchors( ) was used to identify anchors (conserved features) between datasets. TransferData( ) was run to transfer cell type labels described in the published dataset to each nucleus in the snRNA-seq data.

Quality Control and Clustering of snATAC-Seq

The peak-cell counts matrix was loaded and analyzed using R package Signac (V1.7.0). To be included for snATAC-seq analysis, nuclei were required to be present in snRNA-seq data and contain at least 1,000 fragments overlapping with peaks. There were 41,553 nuclei that met these criteria. Term frequency-inverse document frequency normalization was perfumed using RunTFIDF ( ) function and variable peaks were identified using FindTopFeatures ( ). Dimension reduction was performed with singular value decomposition using RunSVD( ) function and Uniform Manifold Approximation and Projection (UMAP) coordinates were calculated using RunUMAP( ). Nuclei clustering was performed using FindClusters( ) based on the top 20 dimensions with the resolution of 0.8.

### Differential Analyses of Gene Expression and Chromatin Accessibility

To identify marker genes and peaks that are enriched in distinct transcriptional subtypes or projections, differential expression/accessibility analysis was performed using findAllMarkers( ) in Seurat/Signac, comparing nuclei from one subtype or projection to all other nuclei. Genes and peaks with FDR <0.05 were reported. To identify genes and peaks that are differentially expressed/accessible between any two subtypes or projections, differential expression/accessibility analysis was performed using findMarkers( ) in Seurat/Signac, comparing nuclei from one subtype or projection to nuclei from another subtype or projection. Genes and peaks with FDR <0.05 were reported.

To identify genes that are differentially expressed in neurons projecting to only one target compared to neurons projecting to multiple targets while controlling for transcriptional cell types, differential expression analysis was performed using FindMarkers( ) in Seurat, comparing nuclei of the same transcriptional cell type expressing only one Projection-TAG to nuclei co-expressing the same Projection-TAG and other TAGs. Cell types that have at least 60 nuclei for each projection group were analyzed, and genes and peaks with FDR <0.05 were reported.

To identify genes and peaks that are differentially regulated by CYP in individual subtypes or projections, differential expression/accessibility analysis was performed using findMarkers( ) in Seurat/Signac, comparing nuclei from CYP-treated animals to nuclei from Saline-treated animals of the same subtype or projection. Genes and peaks with avg_log 2FC >0.5 and FDR <0.05 were reported. To identify IEGs that are activated by CYP in neurons projecting to IT targets, cMOp and cSSp, differential expression analysis was performed using findMarkers( ) in Seurat, comparing activated nuclei, defined by Act-seq, that are positive for the corresponding Projection-TAG from CYP-treated animals to the same number of randomly sampled nuclei (with matched subtype distribution) positive for the same Projection-TAG from Saline-treated animals. IEGs with avg_log 2FC >0.5 and FDR <0.05 were reported.

### Identification of Putative Genomic Regulatory Elements

To identify snATAC-seq peaks that are correlated with gene expression in cis and may act as genomic regulatory elements (GREs) to regulate the expression of target genes, a computational pipeline was modified as described elsewhere. Using snATAC-seq data, the average accessibility of each peak was calculated, and, using snRNA-seq data, the average expression of each gene in individual Subtypes or Nuclei positive for individual TAGs. Pearson correlation coefficient r was then computed between the accessibility of peaks and the expression of genes across Nuclei positive for individual TAGs or Subtypes for any peak-gene pair, in which the center of the peak is located within 5 mbps of the center of the TSS of the gene on the same chromosome. To identify putative GREs that may direct cell type-specific gene expression, pairs of Celltype-specific snATAC-seq peaks (avg_log 2FC >0.5, FDR <0.05, comparing peak accessibility in nuclei of one Cell type to all others) and Cell type-specific snRNA-seq genes (avg_log 2FC >0.5, FDR <0.05, comparing gene expression in nuclei of one T-type to all others) were identified in the same T-type. A peak-gene pair with a strong positive correlation (Pearson's r >0.75) is identified as a putative enhancer (pu.Enhancer) and its putative regulated gene, whereas a pair with a strong negative correlation (Pearson's r<−0.75) is categorized as a putative silencer (pu.Silencer) and its regulated gene. To identify putative GREs that may direct Projection-specific gene expression, pairs of Projection-specific snATAC-seq peaks (avg_log 2FC >0.5, FDR <0.05, comparing peak accessibility in nuclei of one Projection to all others) and Projection-specific snRNA-seq genes (avg_log 2FC >0.5, FDR <0.05, comparing gene expression in nuclei of one Projection to all others) were identified in the same Projection. Projection-specific pu.Enhancers and pu.Silencers and their regulated genes are identified as described above.

### Act-Seq Analysis

To identify cell populations that are activated by visceral pain, the IEG score for each nucleus in snRNA-seq data was calculated using AddModuleScore( ) in Seurat based on the expression of a set of 139 immediate early genes (IEG) previously described. A transcriptional subtype or projection was considered transcriptionally “activated” if the IEG scores of CYP-treated nuclei in this population were significantly higher than the IEG scores of Saline-treated nuclei. A nucleus was considered transcriptionally “activated” if its IEG score was 2 standard deviations higher than the average IEG scores across all Saline-treated nuclei in the same projection group (nuclei expressing the corresponding TAG).

### Statistical Analysis and Visualization

Statistical analyses including the number of animals or cells (n) and p values for each experiment are noted in the figure legends. Statistics and visualization were performed using R version 4.0.1. Student's t-tests were performed using R package stats V4.2.2. ANOVA tests were performed using R package rstatix V0.7.2, followed by post-hoc t-tests with Bonferroni correction. Hypergeometric tests were used to test the significance of the overlap between two gene sets or between nuclei expressing two TAGs using by calling phyper( ) function in R package stats V4.2.2. Plots were generated using R packages ggplot2 V3.4.0, gplots V3.1.3, and UpSetR V1.4.0.

### Data and Code Availability

Raw and processed data of snRNA-seq and snATAC-seq experiments included herein were deposited to the NCBI Gene Expression (GEO) SRA with accession number GSE277718. Custom pipelines and scripts are available on GitHub.

What is claimed is:

1. A Projection-TAG comprising an AAV plasmid, wherein the AAV plasmid comprises:
a promoter;
an RNA barcode;
a fluorescent marker; and
a regulatory element.

2. The Projection-TAG of claim 1, wherein the promoter comprises a chicken beat-actin (CAG) promoter.

3. The Projection-TAG of claim 1, wherein the RNA barcode is unique to a neuron projecting to a target region.

4. The Projection-TAG of claim 1, wherein the RNA barcode is 100 base pairs.

5. The Projection-TAG of claim 1, wherein the fluorescent marker comprises a fluorescent protein fused with a protein targeting a localization domain.

6. The Projection-TAG of claim 5, wherein the fluorescent protein comprises GFP and oScarlet.

7. The Projection-TAG of claim 5, wherein the localization domain targets a nuclear membrane.

8. The Projection-TAG of claim 7, wherein the protein targeting the nuclear membrane is Sun1.

9. The Projection-TAG of claim 1, wherein the regulatory element is a woodchuck hepatitis virus post-transcriptional regulatory element (WPRE).

10. A method for multiplex tracing of a projection neuron in a brain of a subject in need thereof, the method comprising:
administering a Projection-TAG to the subject, the Projection-TAG comprising an AAV plasmid, wherein the AAV plasmid comprises:
a promoter;
an RNA barcode;
a fluorescent marker; and
a regulatory element;
obtaining one or more biological samples from the subject; and
applying an imaging modality to the one or more biological samples.

11. The method of claim 10, wherein the projection neuron comprises a neuron in a primary motor cortex (MOp) and a primary somatosensory cortex (SSp).

12. The method of claim 10, wherein the RNA barcode of the Projection-TAG is unique to the neuron projecting to a target region.

13. The method of claim 12, wherein the RNA barcode is comprised of 100 base pairs.

14. The method of claim 12, wherein the target region comprises an intratelencephalic (IT) target and an extratelencephalic (ET) target.

15. The method of claim 14, wherein the ET target comprises a contralateral MOp (cMOp) and a contralateral SSp (cSSp).

16. The method of claim 14, wherein the ET target comprises a ipsilateral ventral posterior nucleus of the thalamus (VP) region, a ipsilateral periductal grey (PAG) region, a ipsilateral medulla (MY) region, a lumbar spinal cord (SC_{L}) region, and a sacral spinal cord (SC_{S}) region.

17. The method of claim 10, wherein the imaging modality applied to the one or more biological samples is selected from immunofluorescent staining, fluorescence in situ hybridization (FISH), flow cytometry and fluorescence-activated cell sorting (FACS), single-cell RNA-sequencing (scRNA-seq), single-nucleus RNA-sequencing (snRNA-seq), and single-nucleus ATAC-sequencing (snATAC-seq).

18. The method of claim 10, wherein the fluorescent marker comprises a fluorescent protein fused with a protein targeting a localization domain.

19. The method of claim 18, wherein the fluorescent protein comprises GFP and oScarlet.

20. The method of claim 19, wherein the fluorescent protein is photobleached.
